(19) United States
(12) Patent Application Publication
(10) Pub. No.: US 2026/0166182 A1
(43) Pub. Date: Jun. 18, 2026

(54) CYANINE-BASED FLUOROPHORES

(71) Applicants: The General Hospital Corporation, Boston, MA (US); Georgia State University Research Foundation, Inc., Atlanta, GA (US)

(72) Inventors: Hak Soo Choi, Needham, MA (US); Homan Kang, East Boston, MA (US); Maged M. Henary, Lawrenceville, GA (US)

(21) Appl. No.: 19/129,441

(22) PCT Filed: Nov. 14, 2023
(86) PCT No.: PCT/US2023/079600

§ 371 Date: May 13, 2025

Related U.S. Application Data
(60) Provisional application No. 63/425,096, filed on Nov. 14, 2022.

(51) Int. Cl. A61K 49/00; A61P 35/00; C07D 209/12; C07D 405/14; C07D 409/14

(52) U.S. Cl. CPC A61K 49/0032; A61P 35/00; C07D 209/12; C07D 405/14; C07D 409/14

(57) ABSTRACT

The present application provides compounds and methods for structure-inherent targeting and intraoperative imaging of cancerous tumors.

### FEDERALLY SPONSORED RESEARCH OR DEVELOPMENT

This invention was made with Government support under Grant Nos. EB022230 and HL143020 awarded by The National Institutes of Health. The Government has certain rights in the invention.

### TECHNICAL FIELD

This disclosure relates to heptamethine cyanine-based fluorophores useful, e.g., in intraoperative optical imaging and image-guided cancer surgeries.

### BACKGROUND

There are numerous deadly diseases affecting current human population. For example, cancer is one of the leading causes of death in contemporary society. Currently, cancer incidence is nearly 450 cases of cancer per 100,000 men and women per year, while cancer mortality is nearly 71 cancer deaths per 100,000 men and women per year. The socioeconomic burden of cancer is substantial and reflects both healthcare spending as well as lost productivity due to co-morbidities and premature death. Healthcare spending on treating cancer exceed tens of billions of dollars worldwide. However, the economic burden of lost productivity due to cancer is over 60% of the total economic burden associated with cancer. Prevention, early detection, and effective treatment help reduce this economic burden.

### SUMMARY

This disclosure is based, at least in part, on a realization that cyanine-based fluorophores without any chemically conjugated targeting moiety possess structure-inherent tumor targeting (SITT) properties. The experimental data presented in this disclosure demonstrates that heptamethine cyanine-based fluorophores possess not only targetability of tumor microenvironments without the need for additional targeting ligands but also NIR-II imaging capabilities (minimum scattering and ultralow autofluorescence). Without being bound by any theory, the compounds within the present claims selectively accumulate in bone-marrow-derived and/or tissue-resident/tumor-associated immune cells and allow for cancer detection due to the abundance of these immune cells in tumoral tissues. Because the compounds inherently target immune cells in any tumor microenvironment, their use is not limited to any specific cancer cell type. Hence, the compounds provide ubiquitous tumor targetability. As the experimental results in this disclosure demonstrate, the cyanine-based fluorophores within the instant claims allow for high tumor-to-background ratio (TBR) ranging from 9.5 to 47 in pancreatic, breast, and lung cancer mouse models upon a single bolus intravenous injection. Furthermore, and importantly here, the compounds of this disclosure can be used to detect small cancerous tissues smaller than 2 mm in diameter in orthotopic lung cancer models. Hence, the presently claimed compounds are effective cancer-targeting agents which are useful not only in early cancer detection but also in intraoperative optical imaging and image-guided cancer surgery.

Some embodiments provide a compound of Formula (I):

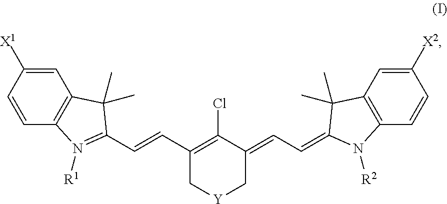

or a pharmaceutically acceptable salt thereof, wherein X¹, R¹, Y, R², and X² are as described herein.

Some embodiments provide a pharmaceutical composition comprising a compound of Formula (I), or a pharmaceutically acceptable salt thereof, and a pharmaceutically acceptable carrier.

Some embodiments provide a method of imaging a cancerous tumor in a subject, the method comprising: (i) administering to the subject an effective amount of a compound of Formula (I), or a pharmaceutically acceptable salt thereof, or a pharmaceutical composition comprising same; (ii) waiting a time sufficient to allow the compound of Formula (I) to accumulate in the cancerous tumor to be imaged; and (iii) imaging the cancerous tumor with a fluorescence imaging technique.

Some embodiments provide a method of treating cancer, the method comprising: (i) imaging a cancerous tumor in a subject according to the method as described herein; and (ii) surgically removing the cancerous tumor from the subject.

Unless otherwise defined, all technical and scientific terms used herein have the same meaning as commonly understood by one of ordinary skill in the art to which the present application belongs. Methods and materials are described herein for use in the present application; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the present application will be apparent from the following detailed description and figures, and from the claims.

### DESCRIPTION OF DRAWINGS

FIG. 1A Intraoperative tumor imaging in the NIR-II window and targeting in the tumor microenvironment (TME). Time-course signal changes in the tumoral tissue and bone marrow in the spine. 50 nmol of SH1 was injected intravenously into the mouse model of pancreatic ductal adenocarcinoma (PDAC). The red circle and dotted line indicate the region of interest. BM; bone marrow. At day 6, black pigment developed in the tumor site which showed relatively low signal intensity. (excitation=808 nm; power density=30 mW cm⁻²; exposure time=50 ms; optical filter=1,070 nm LP)

FIG. 1B Longitudinal monitoring of signal changes in the tumor and bone marrow from the images shown in FIG. 1A.

FIG. 1C SH1 uptake in the bone marrow of spine, sternum, and hindlimb.

FIG. 1D Schematic illustration for two-step mechanism of tumor-targeted SH1 fluorophore: 1) SH1 fluorophore directly targets tumor-associated immune cells, tumor cells as well as myeloblasts/lymphoblasts, 2) targeted myeloblasts/lymphoblasts migrate through blood vessels followed by infiltration to the cancerous region.

FIG. 2A Chemical structure of a TAIC-targeted NIR fluorophore.

FIG. 2B Absorption and NIR-I/II fluorescence emission spectra using silicon (left) and InGaAs (right) CCDs based spectrophotometers of the TAIC-targeted NIR fluorophore of FIG. 2A.

FIG. 2C Charge distribution and polarizability of SH1.

FIG. 2D Physical and optical properties of TME targeting fluorophores. Optical properties were measured at 5 μm SH1 in 5% BSA saline. The quantum yield and fluorescence brightness in the NIR-II region (950-1400 nm) of TME targeting SH1 were compared to those of indocyanine green (ICG).

FIG. 2E Schematic diagram of HOMO and LUMO energy levels of SH1, SH2, SH21, and SH22 calculated by density functional theory. The HOMO and LUMO energy levels were plotted based on the optimized S0 and S1 geometries using Gaussian 16 at Becke, 3-parameter, Lee-Yang-Parr (B3-LYP)/6-31G(d).

FIG. 3A Identification of SH1 targeted immune cells from bone marrow and tumor tissues by flow cytometry analysis. Immune cells including macrophages, monocytes, neutrophils, monocytes, lymphocytes, and dendritic cells (DC) assessed in the isolated bone marrow from mice intravenously injected with SH1 at 24 h post-injection. (n=2-3 for each tissue)

FIG. 3B Identification of SH1 targeted immune cells from bone marrow and tumor tissues by flow cytometry analysis. Immune cells including macrophages, monocytes, neutrophils, monocytes, lymphocytes, and dendritic cells (DC) assessed in the tumor from LLC tumor mice intravenously injected with SH1 at 48 h post-injection. (n=2-3 for each tissue)

FIG. 3C H&E staining compared with NIR fluorescence microscopic images of a resected tumor. Arrowheads indicate SH1 targeted tumor-associated macrophages.

FIG. 3D F4/80 antigen (mouse macrophage marker) staining images compared with NIR fluorescence microscopic images of a resected tumor. Arrowheads indicate SH1 targeted tumor-associated macrophages.

FIG. 4A In vivo imaging of SH1 tumor targetability based on tumor-associated immune cells on lung carcinoma (LLC cell line) bearing C57BL/6 mice compared to IR-780 and ICG as a control. NIR-II fluorescence imaging until 48 h post-injection of SH1 tumor targetability based on tumor-associated immune cells on lung carcinoma (LLC cell line) bearing C57BL/6 mice compared to IR-780 and ICG as a control. (n=4, mean±s.e.m., p values <0.05 were considered significant: * p<0.05) (excitation=808 nm; power density=30 mW cm⁻²; exposure time=15 ms; optical filter=1,070 nm LP).

FIG. 4B In vivo imaging of SH1 tumor targetability based on tumor-associated immune cells on lung carcinoma (LLC cell line) bearing C57BL/6 mice compared to IR-780 and ICG as a control. NIR-II fluorescence imaging until 48 h post-injection of SH1 tumor targetability based on tumor-associated immune cells on lung carcinoma (LLC cell line) bearing C57BL/6 mice compared to IR-780 and ICG as a control after removing skin and ex vivo imaging of tumor and each organ. (n=4, mean±s.e.m., p values <0.05 were considered significant: * p<0.05) (excitation=808 nm; power density=30 mW cm⁻²; exposure time=15 ms except images for ICG; optical filter=1,070 nm LP)

FIG. 4C Signal intensities of tumor site from FIGS. 4A and 4B.

FIG. 4D Abdominal image of mouse injected with IR-780. Arrowhead indicates that IR-780 is circulating in the blood stream such as the inferior vena cava.

FIG. 4E H&E and NIR images of resected tumors. Exposure time=500 ms except images for ICG due to low signal.

FIG. 4F Flow cytometry summary of a portion of targeted cells for each fluorophore from FIG. 16A-C.

FIG. 4G 3D fluorescence emission computed tomography with x-ray CT (FLECT/CT) images for relative quantification of tumor accumulation at 24 h and 48 h post-injection. The shaded area indicates the volume-of-interest (VOI) for quantification. Arrowheads indicate tumors in all images.

FIG. 4H the tumor accumulations of FIG. 4G. (n=2-3, mean±s.e.m.)

FIG. 5A In vivo imaging of SH1 tumor targetability based on tumor-associated immune cells. NIR-II fluorescence imaging of subcutaneous tumor mouse models such as pancreatic ductal adenocarcinoma (PDAC; Pan02 cell), lung carcinoma (LLC cell), and triple-negative breast adenocarcinoma (E0771 cell) with various tumor sizes. 2×10⁵ cells of each cell line (100 μL of DMEM/Matrigel; 50 v/v %) were inoculated in the C57BL/6 mouse flank region, respectively. Female C57BL/6 mice were used for the breast cancer model, otherwise male mice were used. These mice were on active surveillance until diameters of their tumors reached to desired sizes such as ˜5 mm, 10 in mm, and 15 mm, respectively. (excitation=808 nm; power density=30 mW cm⁻²; exposure time=25-50 ms; optical filter=1,070 nm LP).

FIG. 5B In vivo imaging of SH1 tumor targetability based on tumor-associated immune cells. Tumor-to-background signal ratios of dissected tumors compared with muscles (n=6, mean±s.e.m.). Asterisk indicates the same pancreatic tumor in FIG. 5A.

FIG. 6A Orthotopic lung tumor targeting. 50 nmol of SH1 was injected into the mouse model of orthotopic lung cancer tumors 48 h prior to imaging. Arrowheads indicate lung tumors. (excitation=808 nm; power density=30 mW cm⁻²; exposure time=25 ms; optical filter=1,070 nm LP).

FIG. 6B. Orthotopic lung tumor targeting. NIR images of dissected tumors compared with muscle and lung (n=3, mean±s.e.m.)

FIG. 6C. Orthotopic lung tumor targeting. Tumor-to-background signal ratios of dissected tumors compared with muscle and lung (n=3, mean±s.e.m.).

FIG. 6D. Orthotopic lung tumor targeting. Postoperative histopathological examination; H&E staining images and NIR fluorescence microscopic images.

FIG. 7A contains a synthetic scheme showing chemical preparation of exemplified compounds SH1, SH2, SH21, and SH22.

FIG. 7B contains a synthetic scheme showing chemical preparation of O and S-containing exemplified compounds.

FIG. 7C contains a synthetic scheme showing chemical preparation of S-containing exemplified compounds and O-containing exemplified compounds modified with propargyl alcohol.

FIG. 7D contains a synthetic scheme showing chemical preparation of exemplified compounds.

FIG. 8A Chemical structures and physicochemical of SH1, SH2, SH21, and SH22 NIR fluorophores.

FIG. 8B Absorption and emission spectra of SH2, SH21, and SH22 (5 μM) in 5 wt/v % BSA in saline.

FIG. 8C Physicochemical and optical properties of SH series. In silico calculations of logD at pH 7.4 and 3D molecular surface area were calculated using MarvinSketch calculator plug-in (ChemAxon).

FIG. 8D NIR-II fluorescence image of the SH series (5 μM) in 5 wt % BSA in saline using InGaAs CCDs with 1100 LP filter in 808 nm excitation.

FIG. 9A Evaluation of the depth detection capability of SH1 in the NIR-I/II window. Signal-to-background ratio (SBR) comparisons of SH1 (20 μM in 5% BSA saline) using gelatin phantoms with varying depths up to 12 mm under NIR-I (760 nm excitation with 780 nm LP) and NIR-II imaging systems (808 nm excitation with 1100 nm LP). Solid lined and dotted lined boxes indicate the region of interest and background, respectively.

FIG. 9B Pictorial depiction of camera and laser setup for the phantom experiment using Ninox640 Vis-SWIR camera and zoom lens with SWIR coating along with specific LP filters and 808 nm excitation laser.

FIG. 9C SBR plot of SH1 fluorophore with varying depths from FIG. 9A.

FIG. 9D Full width at half maximum (FWHM) plot for SH1 fluorophores with increasing depths from FIG. 9A.

FIG. 9E Cerebral vasculature imaging and intensity profiling in NIR-I (>780 nm using silicon CCD) and NIR-II (>900 nm or >1200 nm using InGaAs CCD) regions.

FIG. 9F Blood vessel network imaging and intensity profiling in the cancerous region using NIR-II beyond 1200 nm.

FIG. 10A A live-cell uptake and inhibition tests for SH1. Live-cell fluorescence imaging of uptake test for SH1 in Pan02 (pancreatic cancer), LLC (lung cancer), E0771 (triple negative breast cancer), and Raw264.7 (macrophage-like) cell lines after incubation with 2 μM of SH1 for 15 min. Exposure time=100 ms. Scale bar=100 μm.

FIG. 10B A live-cell uptake and inhibition tests for SH1. Inhibition test after pretreatment with each cell uptake inhibitor as appropriate followed by incubation with 2 μM of SH1 for 15 min. Exposure time=1 s. Scale bars=100 μm. Exposure time=100 ms. Scale bar=100 μm.

FIG. 10C A live-cell uptake and inhibition tests for SH1. Cytotoxicity test result of SH1 to LLC cells compared to that of IR-780. NIR fluorescence images of LLC cell treated with different concentrations of SH1. Exposure time=100 ms. Scale bar=100 μm.

FIG. 11 Flow cytometry analysis of SH1 uptake in tumor and bone marrow tissues. The tissue was mechanically dissociated and digested with collagenase, DNase and EDTA. Cells were labeled with fluorescence-conjugated antibodies for surface markers. Stained cells were analyzed by flow cytometry. Gating schematic to identify neutrophils, lymphocytes, monocytes, and dendritic cell subsets is shown.

FIG. 12 Comparison of the tumor targetability of SH series. NIR-II images of LLC tumor bearing mice injected with 100 μL of each fluorophore solution (500 μM in 5 wt/v % BSA saline) were obtained at time points 10 min, 24 h, and 48 h using the NIR-II imaging system (808 nm excitation, power density=30 mW cm⁻² exposure time=25 ms, 1100 nm long-pass filter). Abdominal and dissected tumor image for SH2 (indicated with red square) was obtained using the NIR-I fluorescence imaging system (FLARE) due to its low signal intensity in NIR-II region. The yellow arrowhead indicates SH21 is circulating in the blood stream at 48 h post injection.

FIG. 13A NIR-II fluorescence images of SH1, ICG, and IR-780. (excitation=808 nm; power density=30 mW cm⁻²; exposure time=4 ms; optical filter=1,070 nm LP).

FIG. 13B Intensities and concentration curve of physicochemical properties of SH1, ICG, and IR-780 from FIG. 13A.

FIG. 13C Summary table of physicochemical properties of SH1, ICG, and IR-780 from FIG. 13A.

FIG. 14 Biodistribution patterns for SH1, ICG, IR-780 at 4 h post-injection. High nonspecific background signal of IR-780 is because of fast/permanent serum protein binding. Abbreviations used are Du, duodenum; Gb: gallbladder; He, heart; In, intestine; Ki, kidneys; Li, liver; Lu, lungs; Mu, muscle; Pa, pancreas; Sp, spleen.

FIG. 15 Reaction kinetics of SH1, ICG, and IR-780 to serum proteins, mostly albumin. 50 nmol of each dye was mixed with 1 ml of FBS/PBS (pH 7.4) 50/50 v/v %, incubated at 37° C., and analyzed using a BioResolve size exclusion column (200 Å, 2.5 μm, 7.8×300 mm). The binding percentage was calculated by dividing by absorbance of bound dye at 24 h assuming the dyes can bind in maximum capacity in the protein at 24 h. ICG did not show any reactions with proteins until 24 h incubation.

FIG. 16A Identification of SH1, ICG, and IR-780 targeted immune cells from bone marrow by flow cytometry analysis at 48 h post-injection. Immune cells including macrophages, monocytes, neutrophils, monocytes, lymphocytes, and dendritic cells (DC) were assessed in the isolated bone marrow from mice. (n=2-3 for each tissue) The gating strategy is shown in FIG. 11.

FIG. 16B Identification of SH1, ICG, and IR-780 targeted immune cells from tumor tissues by flow cytometry analysis at 48 h post-injection. Immune cells including macrophages, monocytes, neutrophils, monocytes, lymphocytes, and dendritic cells (DC) were assessed in the isolated bone marrow from mice. (n=2-3 for each tissue) The gating strategy is shown in FIG. 11.

FIG. 16C Summary table of FIGS. 16A and 16B.

FIG. 17A 3D tomographic imaging for relative quantification of tumor accumulation. 3D tomographic fluorescence images of LLC tumor-bearing mouse at 24 h post-injection. The shaded area indicates the volume-of-interest (VOI) for quantification. The animal is in the prone position. FLECT/CT: Fluorescence emission computed tomography combined with x-ray CT.

FIG. 17B 3D tomographic imaging for relative quantification of tumor accumulation. 3D tomographic fluorescence images of LLC tumor-bearing mouse at 48 h post-injection. The shaded area indicates the volume-of-interest (VOI) for quantification. The animal is in the prone position. FLECT/CT: Fluorescence emission computed tomography combined with x-ray CT.

FIG. 17C Images of standard samples with concentrations 2.5, 5, and 10 μM in 5 wt % BSA in saline.

FIG. 17D Concentration and intensity curve from the images in FIG. 17C.

FIG. 17E Summary of the quantification results of FIGS. 17A and 17B.

FIG. 18A The tumor targetability of SH1 on PDAC (Pan02 cell line) bearing immune deficient nude mouse (NCr nude homozygous CrTac: NCr-Foxn1^{nu}).

FIG. 18B NIR fluorescence intensity profiles in tumor and skin areas from FIG. 18A.

FIG. 18C Resected organs and tumors. Tumor-to-muscle signal ratios (TMR) were calculated to 3.0 and 3.6. Abbreviations used are Du, duodenum; He, heart; In, intestine; Ki, kidneys; Li, liver; Lu, lungs; Mu, muscle; Pa, pancreas; Sp, spleen. (excitation=808 nm; power density=30 mW cm⁻²; exposure time=200 ms; optical filter=1,100 nm LP).

FIG. 19A Blood concentration decay curve and pharmacokinetic parameters.

FIG. 19B Biodistribution patterns for each resected organ. 1 μmol/kg of SH1 in 5% BSA saline were injected into CD-1 mice, and NIR imaging was carried out at 24 h post-injection. (n=3, mean±s.e.m.) Abbreviations used are Du, duodenum; He, heart; In, intestine; Ki, kidneys; Li, liver; Lu, lungs; Mu, muscle; Pa, pancreas; Sp, spleen.

FIG. 19C Organ-to-muscle intensity ratio for each resected organ of FIG. 19B.

FIG. 20 Physicochemical Properties of Exemplified Compounds Calculated by Using Marvin and JChem Calculator Plug-Ins (ChemAxon).

FIG. 21 Summary of Absorption and Emission Profiles of Exemplified Compounds in Four Different Solvents; (I) EtOH, (II) DMSO, (III) HEPES, and (IV) PBS.

FIG. 22A Theoretical calculations of frontier molecular orbitals for the optimized structures of cyanine compounds based on DFT calculations at the B3LYP/6-311G(d,p) level.

FIG. 22B Theoretical calculations of frontier molecular orbitals for the optimized structures of exemplified compounds based on DFT calculations at the B3LYP/6-311G(d,p) level.

FIG. 23A Molar extinction co-efficient of exemplified compounds.

FIG. 23B Quantum yield of exemplified compounds.

FIG. 23C Molecular brightness of exemplified compounds.

FIG. 24 Photostability data of selected fluorophores compared against commercially available FDA-approved heptamethine cyanine dye ICG.

FIG. 25 Targeting and biodistribution of heptamethine NIR exemplified compounds.

FIG. 26 Targeting Properties and Biodistribution of NIR-Emitting exemplified compounds.

### DETAILED DESCRIPTION

A tumor is not a single component but rather an ensemble production referred to as the tumor microenvironment (TME) composed of stromal fibroblasts, infiltrating immune cells, blood and lymphatic vascular networks, and the extracellular matrix. The TME has emerged as an alternative target in tumor imaging and therapy because the non-tumor cell components are presumably genetically stable which contrasts with tumor cells that are known to be genetically unstable. In addition, many human cancers such as colorectal, gastric, bladder, liver, lung, pancreatic, and cervical cancers are associated with chronic inflammation. In the TME, infiltrating immune cells including inflammatory monocytes are recruited from the circulation and differentiate into macrophages as they migrate into the affected (e.g., inflammatory) tissues when tissues are damaged following infection or injury. Cancerous areas are an example of such inflammatory tissues. Therefore, as the experimental results presented herein show, immune cells including bone marrow-derived and/or tissue-resident/tumor-associated immune cells (TRICs/TAICs) are a principal target for cancer detection due to the abundance of immune cells in tumoral tissues (e.g. tumor-associated macrophages comprise approximately 50% of tumor mass).

Fluorescence imaging in the second NIR spectral window (NIR-II, 1,000-1,700 nm) has recently received tremendous attention in the field of basic molecular imaging for its high clinical applicability due to a significant reduction in tissue autofluorescence and scattering. To date, several organic NIR-II dyes including donor-acceptor-donor (D-A-D) structure-based dyes (e.g. CH1055), FD-1080, Flav7, and conjugated polymers have been reported. However, the lack of fluorophores having a high molecular extinction coefficient and quantum yield with a desirably strong signal brightness, due to the bulkiness of hydrophobic cores and severe fluorescence quenching in aqueous solution, presents a major bottleneck. For this reason, indocyanine green (ICG, the only US FDA-approved heptamethine NIR fluorophore) as well as several other NIR-I dyes have been repurposed for NIR-II fluorescence imaging due to NIR-II emission tail, resulting in even higher signal intensity than commercially available NIR-II dyes (e.g. IR-E1050). However, none of these dyes with NIR-II imaging capability have been reported to target tumors without further modification and/or conjugation of targeting moieties. With targeting moieties, these dyes can only target specific cancer cell types, and the chemical conjugation may also alter the specificity, affinity, and distribution of these agents in cells and tissues.

The present disclosure provides, inter alia, targeted heptamethine cyanine-based fluorophores that possess not only a NIR-II imaging capability but also TAIC-mediated tumor targetability without the conjugation of targeting moieties. The fluorophore compounds of this disclosure possess a high molecular extinction coefficient and quantum yield with a desirably strong signal brightness. The NIR-II capability of these compounds allows for deep tissue imaging in vivo such as bone marrow, cerebral vasculature, and blood vessels in tumors with improved resolution. With TAIC-mediated tumor-targeting, these compounds provide diverse tumor targetability with a high tumor-to-background ratio (TBR). Certain embodiments of these compounds, compositions (e.g., pharmaceutical compositions) containing these compounds, and methods for using these compounds for cancer imaging and image-guided cancer surgeries are described herein.

### Compounds

Some embodiments provide a compound of Formula (I):

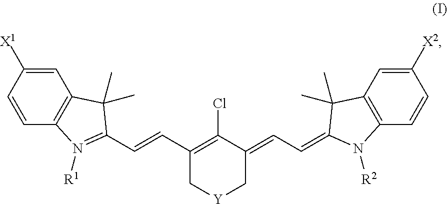

or a pharmaceutically acceptable salt thereof, wherein:
Y is selected from CH₂, O, S, and CHOR₃;
X¹ is selected from H, halo, C₁₋₃ alkyl, C₁₋₃ haloalkyl, OH, C₁₋₃ alkoxy, C₁₋₃ haloalkoxy, NH₂, C₁₋₃ alkylamino, and di(C₁₋₃ alkyl)amino;
X² is selected from H, halo, C₁₋₃ alkyl, C₁₋₃ haloalkyl, OH, C₁₋₃ alkoxy, C₁₋₃ haloalkoxy, NH₂, C₁₋₃ alkylamino, and di(C₁₋₃ alkyl)amino;
R¹ is selected from C₁₋₈ alkyl and C₁₋₈ haloalkyl, wherein said C₁₋₈ alkyl is optionally substituted with OH, C₁₋₃ alkoxy, C(═O)OH, SO₃H, P(═O)(OH)₂, NH₂, C₁₋₃ alkylamino, di(C₁₋₃ alkyl)amino, tri(C₁₋₃ alkyl)amino, C₆₋₁₀ aryl, or 5-10 membered heteroaryl;
R² is selected from C₁₋₈ alkyl and C₁₋₈ haloalkyl, wherein said C₁₋₈ alkyl is optionally substituted with OH, C₁₋₃ alkoxy, C(═O)OH, SO₃H, P(═O)(OH)₂, NH₂, C₁₋₃ alkylamino, di(C₁₋₃ alkyl)amino, tri(C₁₋₃ alkyl)amino, C₆₋₁₀ aryl, or 5-10 membered heteroaryl; and
R³ is selected from C₁₋₈ alkyl and C₂₋₄ alkynyl.

Some embodiments provide a compound of Formula (I):

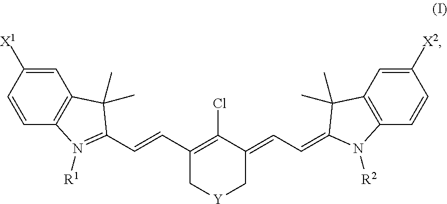

or a pharmaceutically acceptable salt thereof, wherein:
Y is selected from CH₂, O, and S;
X¹ is selected from H, halo, C₁₋₃ alkyl, C₁₋₃ haloalkyl, OH, C₁₋₃ alkoxy, C₁₋₃ haloalkoxy, NH₂, C₁₋₃ alkylamino, and di(C₁₋₃ alkyl)amino;
X² is selected from H, halo, C₁₋₃ alkyl, C₁₋₃ haloalkyl, OH, C₁₋₃ alkoxy, C₁₋₃ haloalkoxy, NH₂, C₁₋₃ alkylamino, and di(C₁₋₃ alkyl)amino;
R¹ is selected from C₁₋₈ alkyl and C₁₋₈ haloalkyl, wherein said C₁₋₈ alkyl is optionally substituted with OH, C₁₋₃ alkoxy, C(═O)OH, SO₃H, P(═O)(OH)₂, NH₂, C₁₋₃ alkylamino, di(C₁₋₃ alkyl)amino, tri(C₁₋₃ alkyl)amino, C₆₋₁₀ aryl, or 5-10 membered heteroaryl; and
R² is selected from C₁₋₈ alkyl and C₁₋₈ haloalkyl, wherein said C₁₋₈ alkyl is optionally substituted with OH, C₁₋₃ alkoxy, C(═O)OH, SO₃H, P(═O)(OH)₂, NH₂, C₁₋₃ alkylamino, di(C₁₋₃ alkyl)amino, tri(C₁₋₃ alkyl)amino, C₆₋₁₀ aryl, or 5-10 membered heteroaryl.

In some embodiments, Y is O or S. In some embodiments, Y is O. In some embodiments, Y is S. In some embodiments, Y is CH₂.

In some embodiments, the compound of Formula (I) has formula:

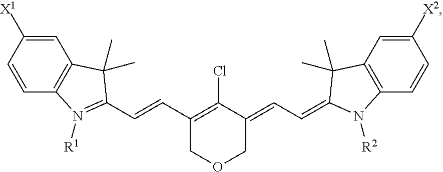

or a pharmaceutically acceptable salt thereof.

In some embodiments, the compound of Formula (I) has formula:

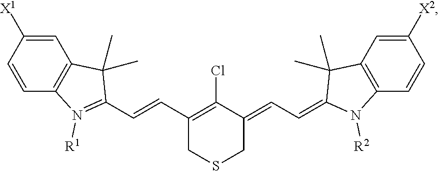

or a pharmaceutically acceptable salt thereof.

In some embodiments, X¹ is halo; and X² is halo. In some embodiments, X¹ is Cl, F, or Br; and X² is Cl, F, or Br. In some embodiments, X¹ is Cl and X² is Cl.

In some embodiments, X¹ is H, F, Cl, or Br; and X² is H, F, Cl, or Br.

In some embodiments, the compound of Formula (I) has formula:

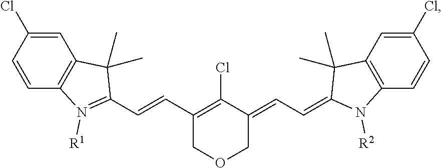

or a pharmaceutically acceptable salt thereof.

In some embodiments, the compound of Formula (I) has formula:

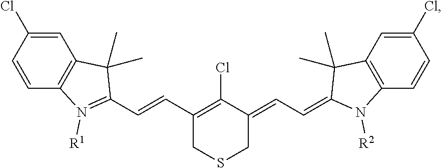

or a pharmaceutically acceptable salt thereof.

In some embodiments, R¹ is C₁₋₈ alkyl. In some embodiments, R¹ is C₁₋₈ haloalkyl. In some embodiments, R¹ is C₁₋₈ alkyl, optionally substituted with OH, C₁₋₃ alkoxy, C(═O)OH, SO₃H, P(═O)(OH)₂, NH₂, C₁₋₃ alkylamino, di(C₁₋₃ alkyl)amino, tri(C₁₋₃ alkyl)amino, C₆₋₁₀ aryl, or 5-10 membered heteroaryl. In some embodiments, R¹ is C₁₋₈ alkyl, optionally substituted with OH, C₁₋₃ alkoxy, C(═O)OH, SO₃H, P(═O)(OH)₂, NH₂, C₁₋₃ alkylamino, di(C₁₋₃ alkyl)amino, or tri(C₁₋₃ alkyl)amino. In some embodiments, R¹ is C₁₋₈ alkyl, optionally substituted with OH, C(═O)OH, SO₃H, or C₆₋₁₀ aryl. In some embodiments, R¹ is C₁₋₆ alkyl.

In some embodiments, R² is C₁₋₈ alkyl. In some embodiments, R² is C₁₋₈ haloalkyl. In some embodiments, R² is C₁₋₈ alkyl, optionally substituted with OH, C₁₋₃ alkoxy, C(═O)OH, SO₃H, P(═O)(OH)₂, NH₂, C₁₋₃ alkylamino, di(C₁₋₃ alkyl)amino, tri(C₁₋₃ alkyl)amino, C₆₋₁₀ aryl, or 5-10 membered heteroaryl. In some embodiments, R² is C₁₋₈ alkyl, optionally substituted with OH, C₁₋₃ alkoxy, C(═O)OH, SO₃H, P(═O)(OH)₂, NH₂, C₁₋₃ alkylamino, di(C₁₋₃ alkyl)amino, or tri(C₁₋₃ alkyl)amino. In some embodiments, R² is C₁₋₈ alkyl, optionally substituted with OH, C(═O)OH, SO₃H, and C₆₋₁₀ aryl. In some embodiments, R² is C₁₋₆ alkyl.

In some embodiments, R¹ is C₁₋₆ alkyl, optionally substituted with C₆₋₁₀ aryl; and R² is C₁₋₆ alkyl, optionally substituted with C₆₋₁₀ aryl.

In some embodiments, R¹ is C₁₋₆ alkyl; and R² is C₁₋₆ alkyl. In some embodiments, R¹ is C₂₋₄ alkyl; and R² is C₂₋₄ alkyl. In some embodiments, R¹ is ethyl or butyl; and R² is ethyl or butyl.

In some embodiments, the compound of Formula (I) is selected from any one of the following compounds:

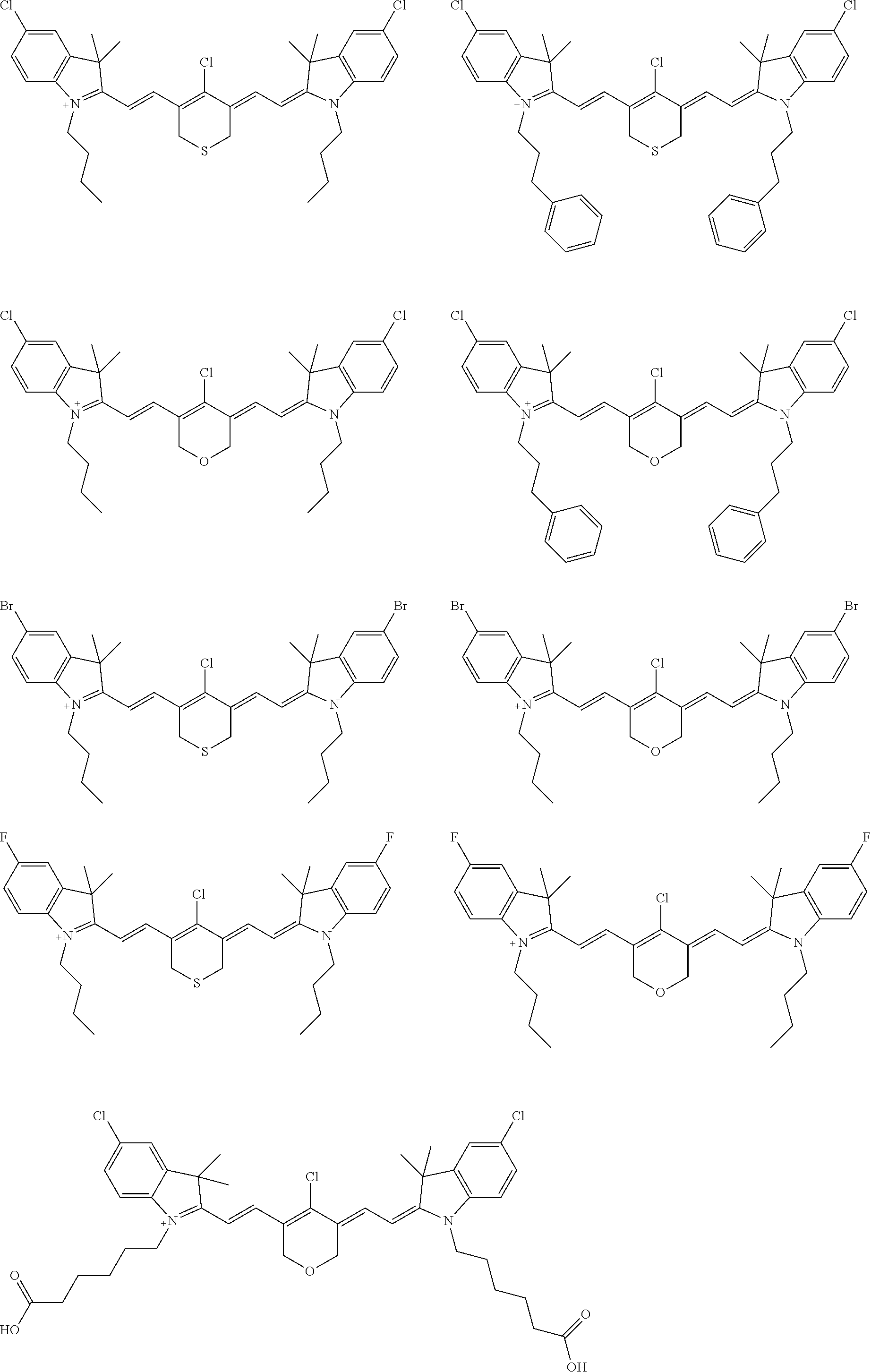

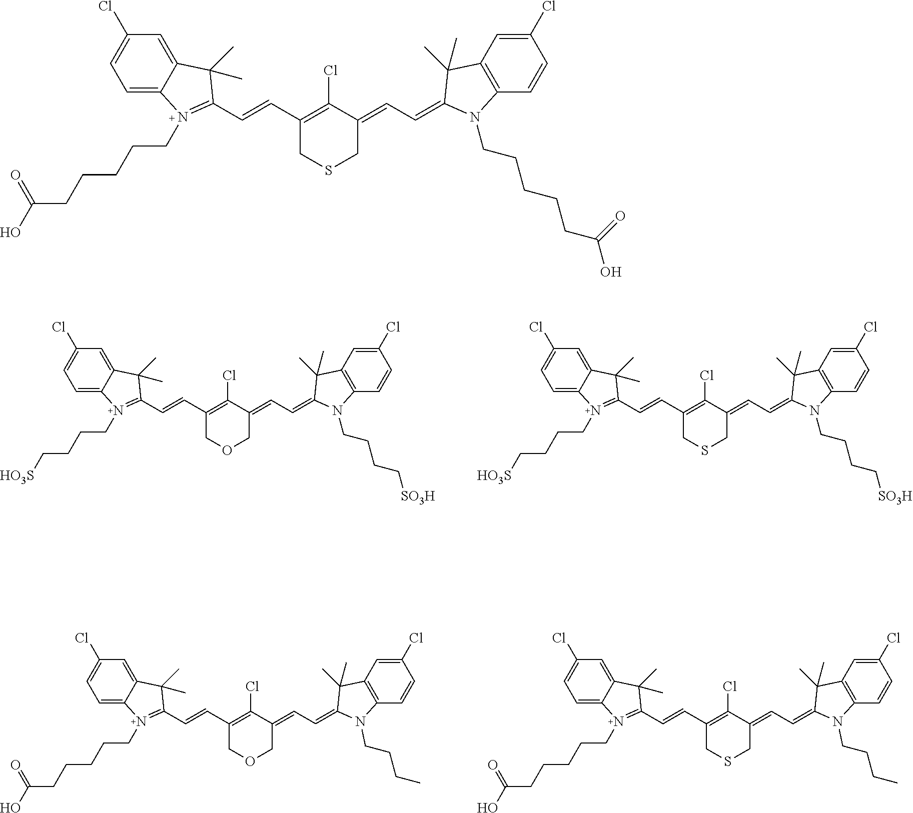

or a pharmaceutically acceptable salt thereof.

In some embodiments, Y is CHOR₃. In some embodiments, R³ is C₂₋₄ alkynyl. In some embodiments, R³ is propargyl. In some embodiments, R³ is propargyl, X¹ is H, F, Cl, or Br; X² is H, F, Cl, or Br: R¹ is ethyl or butyl; and R² is ethyl or butyl.

In some embodiments, the compound of Formula (I) is selected from any one of the following compounds:

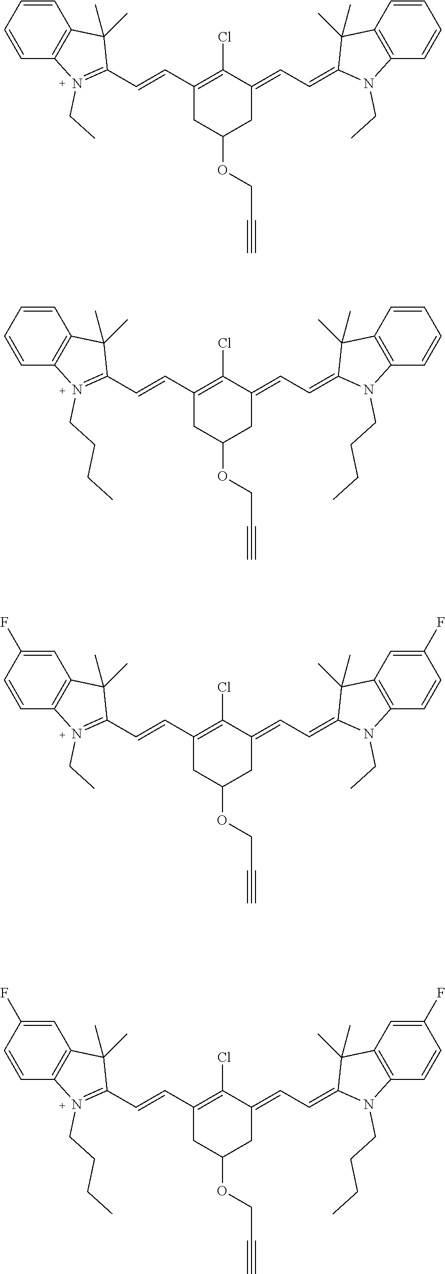

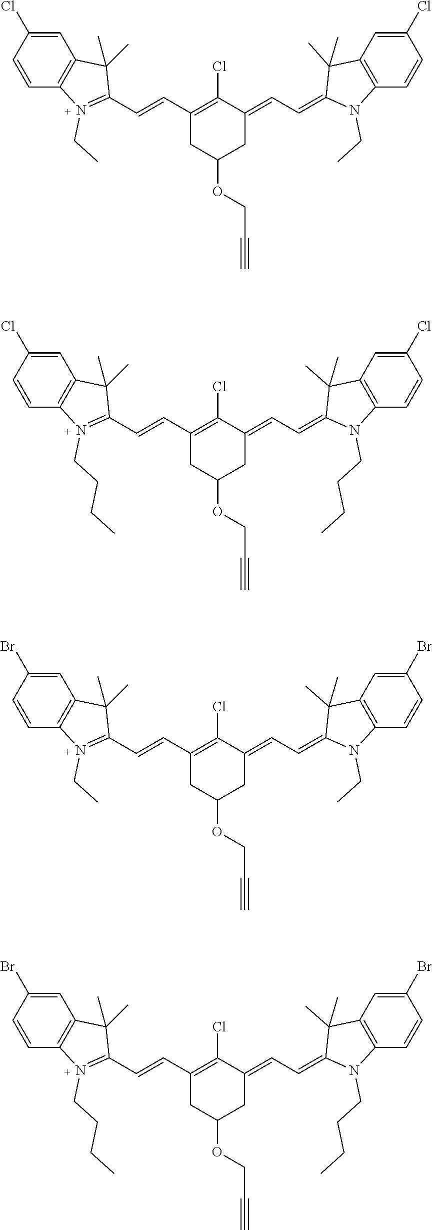

or a pharmaceutically acceptable salt thereof.

Without being bound by any particular theory or speculation, it is believed that the compounds of Formula (I) possess fluorescent properties. In other words, the compounds are capable or emitting electromagnetic radiation or light of a wavelength. In some embodiments, the radiation is visible (e.g., visible light) or invisible (e.g., ultraviolet, infrared, or NIR radiation). In some embodiments, the compounds are capable to absorbing light or radiation of a short wavelength and emitting light or radiation of a longer wavelength. In some embodiments, the emission maximum for the compounds of Formula (I) is within NIR 1 or NIR II wavelength spectrum. In some embodiments, the emission maximum wavelength for the compound of Formula (I) is from about 600 nm to about 1800 nm, from about 600 to about 1000 nm, form about 650 to about 950 nm, from about 950 nm to about 1750 nm, from about 1000 to about 1700 nm, or from 1050 to about 1650 nm. In some embodiments, the emission maximum wavelength for the compound of Formula (I) is about 650 nm, about 700 nm, about 750 nm, about 800 nm, about 900 nm, about 950 nm, about 1000 nm, about 1050 nm, about 1100 nm, about 1150 nm, about 1200 nm, about 1250 nm, about 1300 nm, about 1500, about 1600 nm, about 1650 nm, about 1700 nm, or about 1750 nm. Without being bound by any particular theory of speculation, the emission maximum wavelength in NIR II window advantageously allows to use the compounds of Formula (I) for fluorescent imaging, because the compound's emitted NIR II radiation can be detected by a NIR-II surgical navigation system, a NIR-II camera, a NIR-II confocal/spinning-disc confocal microscope, a NIR-II light sheet microscope, NIR-II two-photon/multiphoton microscope, and NIR-II fluorescence lifetime microscope.

### Pharmaceutically Acceptable Salts

In some embodiments, a salt of a compound of Formula (I) is formed between an acid and a basic group of the compound, such as an amino functional group, or a base and an acidic group of the compound, such as a carboxyl functional group. According to another embodiment, the compound is a pharmaceutically acceptable acid addition salt.

In some embodiments, acids commonly employed to form pharmaceutically acceptable salts of the compounds of Formula (I) include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

In some embodiments, bases commonly employed to form pharmaceutically acceptable salts of the compounds of Formula (I) include hydroxides of alkali metals, including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C₁-C₆)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like.

In some embodiments, the compounds of Formula (I), or pharmaceutically acceptable salts thereof, are substantially isolated.

### Methods of Use

In one general aspect, the present application relates to compounds of formula (I) useful in imaging techniques, diagnosing and monitoring treatment of various diseases and conditions described herein. In some embodiments, because the compounds of Formula (I) can emit light after absorbing light, the compounds are useful in fluorescence imaging or optical imaging. In some embodiments, fluorescence imaging is NIR-II fluorescence imaging. In some embodiments, fluorescent imaging is carried out to detect light emitted by the compound of Formula (I) at a wavelength from about 950 nm to about 1750 nm, from about 1000 nm to about 1700 nm, about 1000 nm, about 1100 nm, about 1200 nm, about 1300 nm, about 1400 nm, about 1500 nm, about 1600 nm, or about 1700 nm.

Fluorescence imaging is a type of non-invasive imaging technique that can help visualize biological processes taking place in a living organism. This imaging technique is very sensitive, allowing to detect fluorophore-containing compounds in biological tissues even at picomolar concentration. The method commonly includes administering exogenously a fluorophore compound to a patient, then exciting the compound by pointing a source of exciting radiation (e.g. light) to a tissue where the compound is expected to be accumulated, the then detecting fluorescent emission at the tissue site.

In some embodiments, the present disclosure provides a method of imaging a tissue (e.g., cancerous tumor) in a subject, the method comprising (i) administering to the subject an effective amount of a compound of Formula (I), or a pharmaceutically acceptable salt thereof, or a pharmaceutical composition of comprising same, (ii) waiting a time sufficient to allow the compound to accumulate in the tissue (e.g., cancerous tumor) to be imaged; and (iii) imaging the tissue (e.g., cancerous tumor) with a fluorescence imaging technique.

In some embodiments, the tissue is selected from epithelial tissue, mucosal tissue, connective tissue, muscle tissue, skin tissue, fibrous tissue, vascular tissue, and nervous tissue. In some embodiments, the tissue is at or near an organ selected from lung, stomach, intestines, liver, thyroid, bladder, heart, eye, skin, kidney, gland, brain, pancreas, colon, lymph node, spleen, and prostate. In some embodiments, the issue is a cancerous tumor tissue. In some embodiments, the cancerous tumor tissue is selected from sarcoma, angiosarcoma, fibrosarcoma, rhabdomyosarcoma, liposarcoma, myxoma, rhabdomyoma, fibroma, lipoma, teratoma, lung cancer, bronchogenic carcinoma squamous cell, undifferentiated small cell, undifferentiated large cell, adenocarcinoma, alveolar bronchiolar carcinoma, bronchial adenoma, sarcoma, lymphoma, chondromatous hamartoma, mesothelioma, gastrointestinal cancer, cancer of the esophagus, squamous cell carcinoma, adenocarcinoma, leiomyosarcoma, lymphoma, cancer of the stomach, carcinoma, lymphoma, leiomyosarcoma, cancer of the pancreas, ductal adenocarcinoma, insulinoma, glucagonoma, gastrinoma, carcinoid tumor, vipoma, cancer of the small bowel, adenocarcinoma, lymphoma, carcinoid tumors, Kaposi's sarcoma, leiomyoma, hemangioma, lipoma, neurofibroma, fibroma, cancer of the large bowel or colon, tubular adenoma, villous adenoma, hamartoma, leiomyoma, genitourinary tract cancer, cancer of the kidney adenocarcinoma, Wilm's tumor (nephroblastoma), lymphoma, leukemia, cancer of the bladder, cancer of the urethra, squamous cell carcinoma, transitional cell carcinoma, cancer of the prostate, cancer of the testis, seminoma, teratoma, embryonal carcinoma, teratocarcinoma, choriocarcinoma, sarcoma, interstitial cell carcinoma, fibroma, fibroadenoma, adenomatoid tumors, lipoma, liver cancer, hepatoma hepatocellular carcinoma, cholangiocarcinoma, hepatoblastoma, angiosarcoma, hepatocellular adenoma, hemangioma, bone cancer, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor, chordoma, osteochrondroma (osteocartilaginous exostoses), benign chondroma, chondroblastoma, chondromyxofibroma, osteoid osteoma giant cell tumor, nervous system cancer, cancer of the skull, osteoma, hemangioma, granuloma, xanthoma, osteitis deformans, cancer of the meninges meningioma, meningiosarcoma, gliomatosis, cancer of the brain, astrocytoma, medulloblastoma, glioma, ependymoma, germinoma (pinealoma), glioblastoma multiforme, oligodendroglioma, schwannoma, retinoblastoma, congenital tumors, cancer of the spinal cord, neurofibroma, meningioma, glioma, sarcoma, gynecological cancer, cancer of the uterus, endometrial carcinoma, cancer of the cervix, cervical carcinoma, pre tumor cervical dysplasia, cancer of the ovaries, ovarian carcinoma, serous cystadenocarcinoma, mucinous cystadenocarcinoma, unclassified carcinoma, granulosa-theca cell tumor, Sertoli Leydig cell tumor, dysgerminoma, malignant teratoma, cancer of the vulva, squamous cell carcinoma, intraepithelial carcinoma, adenocarcinoma, fibrosarcoma, melanoma, cancer of the vagina, clear cell carcinoma, squamous cell carcinoma, botryoid sarcoma, embryonal rhabdomyosarcoma, cancer of the fallopian tubes, hematologic cancer, cancer of the blood, acute myeloid leukemia (AML), chronic myeloid leukemia (CML), acute lymphoblastic leukemia (ALL), chronic lymphoblastic leukemia, chronic lymphocytic leukemia, myeloproliferative diseases, multiple myeloma, myelodysplastic syndrome, Hodgkin's lymphoma, non-Hodgkin's lymphoma (malignant lymphoma), Waldenstrom's macroglobulinemia, skin cancer, malignant melanoma, basal cell carcinoma, squamous cell carcinoma, Kaposi's sarcoma, moles dysplastic nevi, lipoma, angioma, dermatofibroma, keloids, psoriasis, adrenal gland cancer, and neuroblastoma.

In some embodiments, the time sufficient to allow the compound of Formula (I) to accumulate in the cancerous tumor is from about 24 hours to about 168 hours, from about 48 hours to about 96 hours, about 24 hours, about 48 hours, about 72 hours, about 96 hours, about 120 hours, about 144 hours, or about 168 hours.

In some embodiments, the fluorescence imaging technique is NIR-II fluorescence imaging. The imaging can be carried out using a NIR camera, imaging goggles, or telescope, or a similar device. In some embodiments, the device contains a source of light or irradiation to excite the fluorophore.

In some embodiments, the present disclosure provides a method of diagnosing (or early detection) of a disease or disorder (e.g., any of the cancers described herein). The method may include imaging the tissue (e.g., cancerous tissue) as described herein. For example, the method can include (i) administering to the subject an effective amount of a compound of Formula (I), or a pharmaceutically acceptable salt thereof, or a pharmaceutical composition comprising same; (ii) waiting a time sufficient to allow the compound to accumulate in the tissue (e.g., cancerous tumor), and (iii) imaging the tissue with an imaging technique.

In some embodiments, the present disclosure provides a method of treating cancer (any of the cancers described herein), the method comprising: (i) imaging a cancerous tumor in a subject according to an imaging method as described herein; and (ii) surgically removing the cancerous tumor from the subject. In some aspects of these embodiments, the present disclosure provides a method for intraoperative optical and/or fluorescence imaging and image-guided cancer surgery. The imaging can be carried out as described herein, e.g., using NIR camera or vision goggles. Before imaging, the method may include administering a cancer-targeting fluorophore of Formula (I) and then waiting a sufficient amount of time (e.g., 24 hours, 48 hours, 96 hours, or more) for the cancer-targeting compound to accumulate in the cancerous tissue. Suitable examples of cancer surgeries include staging surgery, tumor removal, debulking surgery, palliative surgery, reconstructive surgery, preventive surgery, laparoscopic surgery, laser surgery, cryosurgery, Mohs surgery, and endoscopy.

In some embodiments, the present disclosure provides a method of monitoring treatment of cancer in a subject, the method comprising (i) administering to the subject an effective amount of a compound of Formula (I), or a pharmaceutically acceptable salt thereof, or a pharmaceutical composition comprising same, (ii) waiting a time sufficient to allow the compound of Formula (I) to accumulate in cancerous tumor of the subject; (iii) imaging the cancerous tumor of the subject with an imaging technique; and (iv) administering to the subject a therapeutic agent in an effective amount to treat the cancer. In some embodiments, the method further includes step (v) after (iv), administering to the subject an effective amount of a compound of Formula (I), or a pharmaceutically acceptable salt thereof, or a pharmaceutical composition comprising same; (vi) waiting a time sufficient to allow the compound of Formula (I) to accumulate in the cancerous tumor of the subject; (vii) imaging the cancerous tumor of the subject with an imaging technique; and (viii) comparing the image of step (iii) and the image of step (vii). In one example, comparing the images is indicative of successful treatment of the cancer. Suitable examples of therapeutic agents useful to treat cancer include abarelix, aldesleukin, alemtuzumab, alitretinoin, allopurinol, altretamine, anastrozole, arsenic trioxide, asparaginase, azacitidine, bevacizumab, bexarotene, bleomycin, bortezombi, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panitumumab, pegaspargase, pegfilgrastim, pemetrexed disodium, pentostatin, pipobroman, plicamycin, procarbazine, quinacrine, rasburicase, rituximab, ruxolitinib, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinostat and zoledronate.

### Compositions, Formulations, and Routes of Administration

The present application also provides pharmaceutical compositions comprising an effective amount of a compound of the present disclosure disclosed herein, or a pharmaceutically acceptable salt thereof; and a pharmaceutically acceptable carrier. The pharmaceutical composition may also comprise any one of the additional therapeutic agents described herein. In certain embodiments, the application also provides pharmaceutical compositions and dosage forms comprising any one the additional therapeutic agents described herein. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of the present application include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

The compositions or dosage forms may contain any one of the compounds and therapeutic agents described herein in the range of 0.005% to 100% with the balance made up from the suitable pharmaceutically acceptable excipients. The contemplated compositions may contain 0.001%-100% of any one of the compounds and therapeutic agents provided herein, in one embodiment 0.1-95%, in another embodiment 75-85%, in a further embodiment 20-80%, wherein the balance may be made up of any pharmaceutically acceptable excipient described herein, or any combination of these excipients.

### Routes of Administration and Dosage Forms

The pharmaceutical compositions of the present application include those suitable for any acceptable route of administration. The composition can be administered by any route by which a fluorophore is effectively administrable and that facilitates imaging of a tumorous tissue. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intranasal, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal.

Compositions and formulations described herein may conveniently be presented in a unit dosage form, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, MD (20th ed. 2000). Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

Compositions, formulations, and dosage forms suitable for parenteral administration include aqueous and non-aqueous sterile injection solutions or infusion solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, saline (e.g., 0.9% saline solution) or 5% dextrose solution, immediately prior to use. Extemporaneous injection solutions and suspensions may be prepared from sterile powders, granules and tablets. The injection solutions may be in the form, for example, of a sterile injectable aqueous or oleaginous suspension. This suspension may be formulated according to techniques known in the art using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

### Dosages and Regimens

In the pharmaceutical compositions of the present application, a compound of the present disclosure is present in an effective amount (e.g., a therapeutically effective amount). Effective doses of the imageable compounds may vary, depending on the diseases treated, the severity of the disease, the route of administration, the sex, age and general health condition of the subject, excipient usage, the possibility of co-usage with other imaging agents or therapeutic treatments such as use of other agents and the judgment of the treating physician, lab technician, or a diagnostician.

In some embodiments, an effective amount of the compound can range, for example, from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 2 mg/kg; from about 0.1 mg/kg to about 1 mg/kg; or from about 0.1 mg/kg to about 0.5 mg/kg). In some embodiments, an effective amount of a compound is about 0.1 mg/kg, about 0.5 mg/kg, about 1 mg/kg, about 2 mg/kg, or about 5 mg/kg.

The foregoing dosages can be administered as needed for imaging, for example, on a daily basis (e.g., as a single dose or as two or more divided doses, e.g., once daily, twice daily, thrice daily) or non-daily basis (e.g., every other day, every two days, every three days, once weekly, twice weekly, once every two weeks, once a month).

### Kits

The present invention also includes kits useful, for example, in the imaging and/or treatment of disorders, diseases and conditions referred to herein, which include one or more containers containing a pharmaceutical composition comprising an effective amount of a compound of the present disclosure. Such kits can further include, if desired, one or more of various conventional pharmaceutical kit components, such as, for example, containers with one or more pharmaceutically acceptable carriers, additional containers, etc. Instructions, either as inserts or as labels, indicating quantities of the components to be administered, guidelines for administration, and/or guidelines for mixing the components, can also be included in the kit.

### Definitions

As used herein, the term “about” means “approximately” (e.g., plus or minus approximately 10% of the indicated value).

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

At various places in the present specification various aryl, heteroaryl, cycloalkyl, and heterocycloalkyl rings are described. Unless otherwise specified, these rings can be attached to the rest of the molecule at any ring member as permitted by valency. For example, the term “a pyridine ring” or “pyridinyl” may refer to a pyridin-2-yl, pyridin-3-yl, or pyridin-4-yl ring.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

The term “aromatic” refers to a carbocycle or heterocycle having one or more polyunsaturated rings having aromatic character (i.e., having (4n+2) delocalized π (pi) electrons where n is an integer).

The term “n-membered” where n is an integer typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

As used herein, the phrase “optionally substituted” means unsubstituted or substituted. The substituents are independently selected, and substitution may be at any chemically accessible position. As used herein, the term “substituted” means that a hydrogen atom is removed and replaced by a substituent. A single divalent substituent, e.g., oxo, can replace two hydrogen atoms. It is to be understood that substitution at a given atom is limited by valency.

Throughout the definitions, the term “Cₙ₋ₘ” indicates a range which includes the endpoints, wherein n and m are integers and indicate the number of carbons. Examples include C₁₋₄, C₁₋₆, and the like.

As used herein, the term “Cₙ₋ₘ alkyl”, employed alone or in combination with other terms, refers to a saturated hydrocarbon group that may be straight-chain or branched, having n to m carbons. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used herein, “Cₙ₋ₘ alkynyl,” employed alone or in combination with other terms, refers to an alkyl group having one or more carbon-carbon triple bonds. Example alkynyl groups include, but are not limited to, ethynyl, propyn-1-yl, propyn-2-yl (propargyl), and the like. In some embodiments, the alkynyl moiety contains 2 to 4 carbon atoms.

As used herein, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, wherein the alkyl group has n to m carbon atoms. In some embodiments, the haloalkyl group is fluorinated only. In some embodiments, the alkyl group has 1 to 6, 1 to 4, or 1 to 3 carbon atoms.

As used herein, the term “Cₙ₋ₘ alkoxy”, employed alone or in combination with other terms, refers to a group of formula —O-alkyl, wherein the alkyl group has n to m carbons. Example alkoxy groups include, but are not limited to, methoxy, ethoxy, propoxy (e.g., n-propoxy and isopropoxy), butoxy (e.g., n-butoxy and tert-butoxy), and the like. In some embodiments, the alkyl group has 1 to 6, 1 to 4, or 1 to 3 carbon atoms.

As used herein, “Cₙ₋ₘ haloalkoxy” refers to a group of formula —O-haloalkyl having n to m carbon atoms. An example haloalkoxy group is OCF₃. In some embodiments, the haloalkoxy group is fluorinated only. In some embodiments, the alkyl group has 1 to 6, 1 to 4, or 1 to 3 carbon atoms.

As used herein, the term “amino” refers to a group of formula —NH₂.

As used herein, the term “Cₙ₋ₘ alkylamino” refers to a group of formula —NH(alkyl), wherein the alkyl group has n to m carbon atoms. In some embodiments, the alkyl group has 1 to 6, 1 to 4, or 1 to 3 carbon atoms. Examples of alkylamino groups include, but are not limited to, N-methylamino, N-ethylamino, N-propylamino (e.g., N-(n-propyl)amino and N-isopropylamino), N-butylamino (e.g., N-(n-butyl)amino and N-(tert-butyl)amino), and the like.

As used herein, the term “di(Cₙ₋ₘ-alkyl)amino” refers to a group of formula-N(alkyl)₂, wherein the two alkyl groups each has, independently, n to m carbon atoms. In some embodiments, each alkyl group independently has 1 to 6, 1 to 4, or 1 to 3 carbon atoms.

As used herein, “halo” refers to F, Cl, Br, or I. In some embodiments, a halo is F, Cl, or Br.

As used herein, the term “aryl,” employed alone or in combination with other terms, refers to an aromatic hydrocarbon group, which may be monocyclic or polycyclic (e.g., having 2, 3 or 4 fused rings). The term “Cₙ₋ₘ aryl” refers to an aryl group having from n to m ring carbon atoms. Aryl groups include, e.g., phenyl, naphthyl, anthracenyl, phenanthrenyl, indanyl, indenyl, and the like. In some embodiments, aryl groups have from 6 to 10 carbon atoms. In some embodiments, the aryl group is phenyl or naphthyl.

As used herein, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur and oxygen. In some embodiments, any ring-forming N in a heteroaryl moiety can be an N-oxide. In some embodiments, the heteroaryl is a 5-10 membered monocyclic or bicyclic heteroaryl having 1, 2, 3 or 4 heteroatom ring members independently selected from nitrogen, sulfur and oxygen. In some embodiments, the heteroaryl is a 5-6 monocyclic heteroaryl having 1 or 2 heteroatom ring members independently selected from nitrogen, sulfur and oxygen. In some embodiments, the heteroaryl is a five-membered or six-membered heteroaryl ring. A five-membered heteroaryl ring is a heteroaryl with a ring having five ring atoms wherein one or more (e.g., 1, 2, or 3) ring atoms are independently selected from N, O, and S. Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms wherein one or more (e.g., 1, 2, or 3) ring atoms are independently selected from N, O, and S. Exemplary six-membered ring heteroaryls are pyridyl, pyrazinyl, pyrimidinyl, triazinyl and pyridazinyl.

The term “compound” as used herein is meant to include all stereoisomers, geometric isomers, tautomers, and isotopes of the structures depicted. Compounds herein identified by name or structure as one particular tautomeric form are intended to include other tautomeric forms unless otherwise specified.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, N═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. In some embodiments, the compound has the (R)-configuration. In some embodiments, the compound has the(S)-configuration.

Compounds provided herein also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “individual”, “patient”, or “subject” used interchangeably, refers to any animal, including mammals, preferably mice, rats, other rodents, rabbits, dogs, cats, swine, cattle, sheep, horses, or primates, and most preferably humans.

As used herein, the phrase “effective amount” or “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

As used herein, the term “preventing” or “prevention” of a disease, condition or disorder refers to decreasing the risk of occurrence of the disease, condition or disorder in a subject or group of subjects (e.g., a subject or group of subjects predisposed to or susceptible to the disease, condition or disorder). In some embodiments, preventing a disease, condition or disorder refers to decreasing the possibility of acquiring the disease, condition or disorder and/or its associated symptoms. In some embodiments, preventing a disease, condition or disorder refers to completely or almost completely stopping the disease, condition or disorder from occurring.

### EXAMPLES

### Materials and Methods

Material: All chemicals and solvents were purchased from Fisher Scientific (Pittsburgh, PA, USA), Sigma-Aldrich (Saint Louis, MO), Combi-Blocks (San Diego, CA) and Acros Organics. TLC plates having silica gel 60 F₂₅₄ (Merck EMD Millipore, Darmstadt, Germany) were used to guide the completion of the reaction. Flash column chromatographic technique was used to purify the final heptamethine dyes by using 60-200 μm, 60 Å classic column silica gel (Dynamic Adsorbents, Norcross, GA). Both ¹H NMR and ¹³C NMR spectra were obtained using high quality Kontes NMR tubes (Kimble Chase, Vineland, NJ) rated to 500 MHz and were recorded on a Bruker Avance (400 MHz) spectrometer using Chloroform-d or DMSO-d₆ containing tetramethylsilane (TMS) as an internal calibration standard set to 0.0 ppm. NMR abbreviations used throughout the experimental section are as follows, s=singlet, d=doublet, t=triplet, q=quartet, p=pentet, m=multiplet, dd=doublet doublets, and bs=broad singlet. UV-Vis/NIR absorption spectra were recorded on a Varian Cary 50 spectrophotometer. High-resolution accurate mass spectra (HRMS) were obtained at the GSU Mass Spectrometry Facility.

Flow cytometry analysis: Bone marrow tissue was mechanically dissociated and digested with collagenase D (175 unit/mL, Roche) and DNase I (100 unit/mL, Roche) followed by EDTA (2 mM, Invitrogen). Erythrocytes were removed using RBC Lysis Buffer (Invitrogen). Tumor tissue was mechanically dissociated and digested with collagenase I (175 unit/mL, Thermo-fisher), Collagenase IV (250 unit/mL, Thermo-fisher, 1700019), and DNase I (100 unit/mL) followed by EDTA (2 mm). Immune cells were isolated from cell suspensions of the bone marrow or tumor tissue using Ficoll® Paque Plus (GE Healthcare). Isolated cells were stained with anti-mouse F4/80 (BM8, Biolegend, 123131), anti-mouse Ly-6G (1A8, Biolegend, 127639), anti-mouse Ly-6C(HK1.4, Biolegend, 128011), anti-mouse CD11c (N418, Biolegend, 117313), anti-mouse CD103 (2E7, Biolegend, 121425), anti-mouse I-A/I-E (M5/114.15.2 Biolegend, 107611), anti-mouse/human CD11b (M1/70, Biolegend, 101254) and anti-mouse CD45 antibodies (30-F11, Biolegend, 103105) conjugated to fluorophores. Isolated cells were stained with Live/dead®-Aqua (Life Technologies) to distinguish live cells. Stained cells were fixed using 1% paraformaldehyde prior to analysis. Data acquisition was performed on a Fortessa cytometer (BD) followed by analysis on FlowJo software (Tree Star).

General NIR-II fluorescence imaging: A 640×512-pixel InGaAs camera (Ninox640, Raptor Photonics) and macro zoom lens (0-10×; Navitar Zoom 7000 with SWIR coating) along with 900, 1070, 1100, and 1200 nm long-pass (LP) filters from either Thorlab or Edmund optics were used to collect the NIR-II signal. InGaAs camera played a role as an open-air imaging system along with the support of the FLARE system. An 808 nm fiber-coupled diode laser (30-35 mW/cm⁻² at the sample) was used as an excitation source. Data acquisition was done using in-house developed software. The mice fur on the region of interest was shaved prior to imaging using a clipper and removed completely using a depilatory cream. 100 μL of SH1 in 5 w/v % BSA saline (500 μm) was injected through the tail vein. Mice were imaged with intact skin using InGaAs camera. At least 3 mice were analyzed for each sample.

Tumor-bearing mouse models: Animals were housed in an AAALAC-certified facility and were studied under the protocols approved by the MGH IACUC (2016N000136). Mice were maintained under anesthesia with isoflurane and oxygen during the experiment. To establish the various tumor-bearing mouse models including lung carcinoma, pancreatic ductal adenocarcinoma (PDAC), and triple negative breast adenocarcinoma, eight-week-old C57BL/6 mice (Taconic Farms, Germantown, NY) were subcutaneously injected with 2×10⁵ LLC, Pan02, and E0771 cells suspended in DMEM/Matrigel (100 μL, 50 v/v %) in the flank region, respectively. Female C57BL/6 mice were used for the breast cancer (E0771) model, otherwise male mice were used. An orthotopic lung cancer model was established in C57BL/6 mice by inoculation of LLC cell suspension (20 μL, 106 in 100 μl DMEM media) after intratracheal intubation.

Tumor histopathology evaluation: The tumor tissue was fixed in 10% neutral buffered formalin and was dehydrated in ethanol, embedded in paraffin, and sectioned into slices (5 μm). After rinsing with PBS, the fixed sections were counterstained with nuclear fast red, dehydrated by ethanol, transferred into xylene, and finally mounted according to the standard protocol. To determine the tissue distribution of the NIR fluorophore, the dissected tissues were embedded in Tissue-Tek optimum cutting temperature compound (Sakura Finetek, Torrance, CA) without a pre-fixation step. The tissue block was frozen at −80° C. Frozen sections were cut at a thickness of 10 or 50 μm by a cryostat (Leica, Germany). Then, the tissue sections were stained with hematoxylin and eosin (H&E). The BioTek Cytation 5 (Winooski, VT) was used for pathological fluorescence imaging and observation.

Quantitative analysis using InSyTe FLECT/CT: 3D fluorescence images were acquired with the TriFoil Imaging InSyTe FLECT/CT, a commercial preclinical imaging system with full 3D fluorescence tomography imaging capability and an inline CT system. Animals were anesthetized with isoflurane during scans. FLECT scans were performed with 116 projections per slice, 780 nm laser for excitation, and 853/45 bandpass fluorescence emission filter. After data collection, fluorescence images were reconstructed using a proprietary FLECT reconstruction engine. CT scans were performed with 360 projections per slice, 35 kV tube voltage, 950 uA tube current, and 100 ms exposure. CT images were reconstructed using the COBRA filtered back-projection algorithm (Exxim, Computing Corporation, Pleasanton, CA). Quantitative analysis of 3D fluorescence images was performed using the VivoQuant 2020 image analysis software (Invicro, Boston, MA). FLECT and CT images were co-registered in VivoQuant 2020. Volumes-of-interest (VOI) were segmented via intensity thresholding, where the threshold limits were set to the local full-width-half-maximum (FWHM) intensity value for the lower threshold bound and local maximum intensity value for the upper threshold bound. Instrument linear response was demonstrated by performing sequential scans of a tissue-mimicking phantom filled with increasing concentrations of a control fluorescent dye (SH1) and performing the VOI analysis above.

Statistical analysis: The fluorescence and background intensities of a region of interest over each tissue were quantified using customized imaging software and ImageJ v1.48 (National Institutes of Health, Bethesda, MD). The signal-to-background ratio (SBR) was calculated as SBR=fluorescence/background, where background was the fluorescence intensity of muscle. Data were reported as mean±s.e.m. with a minimum of three biological replicates. Student's t-test statistical analysis was performed to evaluate the significance of the experimental data. The differences among groups were determined using one-way ANOVA analysis to assess the statistical differences among more than two groups. Ap value of less than 0.05 was considered significant. The data was indicated with *p<0.05, ** p<0.01, *** p<0.001, and **** p<0.0001.

Fluorescence Quantum yield measurement and physicochemical properties: The absorption and emission spectra of fluorophores were measured in 5% BSA saline using UV/Vis/NIR spectrometers (USB2000+VIS+NIR+ES and Flame-NIR, Ocean Insight, Dunedin, FL). Fluorescence emission spectra were collected by NIR excitation with an 808-nm laser. To calculate NIR-II fluorescence quantum yield, the following equation was used:

$${QY}_{SH 1} = {QY}_{ref} \times \frac{{slope}_{sample}}{{slope}_{ref}} \times {( \frac{{n}_{sample}}{{n}_{ref}} )}^{2}$$

where QYₛₐₘₚₗₑ is the QY of SH1, QY_{ref} is the QY of ICG in 30% FBS supplemented with 50 mM HEPES, nₛₐₘₚₗₑ and nref are the refractive indices of SH1 and ICG solutions which are both water (1.33) in this case. In silico calculations of molecular weight, the distribution coefficient (LogD at pH 7.4), surface molecular charge, polarizability, and 3D molecular surface area were performed using MarvinSketch (ChemAxon, Budapest, Hungary).

Theoretical calculations of HOMO and LUMO energy levels of SH series: All the quantum chemical calculations were performed using Gaussian 16 software. The geometry optimizations of the fluorophores were performed using density functional theory (DFT) with Becke's three-parameter hybrid exchange function with Lee-Yang-Parr gradient-corrected correlation functional (B3-LYP functional) and 6-31G(d) basis set. Solvent (water) effects were considered by means of the polarizable continuum model (PCM) method. No constraints to bonds/angles/dihedral angles were applied in the calculations, and all atoms were free to optimize.

In vitro tissue penetration study with gelatin phantom: A 10 mM stock solution of SH1 was diluted in 5% BSA saline to obtain a resulting concentration of 15 μM. Then the diluted solution was filled into a glass capillary tube (0.5 mm in diameter) and the ends of the tube were sealed with clay. To make gelatin phantoms, 10 w/v % gelatin powder was added in 1% intralipid solution and vortexed to completely mix. The mixture was transferred to each well in a 24 well-plate depending on desired depths, and the well-plate was placed in a vacuum chamber for 1 h to remove bubbles. The final well-plate was placed at room temperature overnight. The capillary was placed below the 24-well plate with different depths of gelatin phantom in such a way that it was centered. Captured images were then analyzed and processed using Fiji ImageJ software to plot the signal intensity and full width at half maximum.

Cytotoxicity study: 200 μL of cell suspension was added into a 96-well plate to have 5000 cells in each well. The plate was pre-incubated for 24 hours in a humidified incubator for cells to attach on the bottom of the plate. Stock solutions of SH1, IR-780, and DMSO were premixed with the cell culture media (DMEM containing 10% FBS) to 50 μM, and premixed solutions were filtered using a syringe filter (0.22 nm), and then were diluted to final concentrations of 1-25 μM. After cells reached the desired density (40-50% confluency), the existing media was removed and exchanged with the above solutions at certain concentrations. The plate was further incubated for 24 h. The cell proliferation was determined using a CCK-8 Kit.

In vitro inhibition study: LLC and Raw264.7 cells were seeded and incubated at a density of 20,000 cells/plate in a 24-well plate. To determine the role of membrane transporters and endocytosis in SH1 uptake, the cells were pretreated with 150 mM of 2-deoxyglucose (2DG, glycolysis inhibitor, Sigma-Aldrich, D8375) for 45 min, 1 μM of oligomycin (ATP synthase inhibitor, AdipoGen, AG-CN2-0517-M005, San Diego, CA) for 45 min, 25 μM of Pitstop 2 (clathrin-mediated endocytosis inhibitor, Abcam, 120687) for 45 min, 30 μM of Dyngo 4a (dynamin-dependent endocytosis inhibitor, Abcam, 120689) for 30 min, 250 μM of bromsulphthalein (BSP, organic anion transporter peptides inhibitor, Sigma-Aldrich, 167207) for 5 min, 10 μM of cyclosporin A (OATP inhibitor, Cayman, 12088) for 5 min, 10 μM of MK-571 (ATP-binding cassette transporters inhibitor, Cayman, 10029) for 5 min or 25 μM of D22 (1,1′-Diethyl-2,2′-cyanine iodide, organic cation transporters inhibitor, Sigma-Aldrich, 323764) for 10 min. Cells were then incubated with 2 μM of SH1 at 37° C. for 15 min. The cell viability assay showed that SH1 did not affect cells in concentrations up to 10 μM. After washing twice with HBSS buffer, the cells were imaged using a Nikon TE2000 epifluorescence microscope (TE2000U; Nikon) equipped with a 75-W xenon light source, NIR-compatible optics, and a NIR-compatible 4×, 10×, and 20× Plan Fluor objective lens (Nikon, Melville, NY) with a customized filter set.

In vivo biodistribution and pharmacokinetics of SH dyes: Animals were housed in an AAALAC-certified facility and were studied under the supervision of MGH IACUC in accordance with the approved institutional protocol. Six-week-old CD-1 mice (male; 25-30 g) were purchased from Charles River Laboratories (Wilmington, MA). Mice were maintained under anesthesia with isoflurane and oxygen during the preparation prior to injection. The end of the tail was cut for blood collection. Before injection, blood was sampled in capillary tubes (Fisher Scientific, Pittsburgh, PA) as a reference. Mice were injected with 1 μmol/kg of SH1 in 5% BSA saline, and blood was collected in capillary tubes at the following time points (1, 3, 5, 10, 30, 60, 120, 180, 240 min, and 24 h) to calculate elimination blood half-life (t_{1/2}) values. Mice were imaged using the in-house built NIR-II imaging system. After 24 h post-injection, mice were sacrificed to image organs. At least 3 mice were analyzed for each sample. Results were presented as an exponential decay curve using Prism software version 9.0.2 (GraphPad, San Diego, CA).

Optical Study, Photostability, Physicochemical Properties, and DFT Calculation: 1 mM stock solution in DMSO of the synthesized fluorophores was prepared before starting any spectral measurement. Optical properties for the synthesized fluorophores were measured in four different solvents (EtOH, DMSO, HEPES, and PBS). Absorbance spectra were measured in a Varian Cary 50 absorbance spectrophotometer (190-1100 nm). Fluorescence emission and photostability were measured and emission spectra were recorded on a Shimadzu RF-5301PC spectrofluorometer. Physicochemical properties (Log D, molecular mass, TPSA, H bond D/A, polarizability, molecular volume, and rotatable bonds) were predicted by ChemAxon (JChem plugin). Theoretical calculations of frontier molecular orbitals (FMOs) for the optimized structures of selected fluorophores were calculated based on DFT calculations at the B3LYP/6-311G(d,p) level.

In Vivo Biodistribution Study of NIR Fluorophores: Under the supervision of the MGH IACUC, animals were housed in an AAALAC-certified facility and studied according to institutional protocol #2016N000136. In preparation for injection, CD-1 mice (25-30 g, 6 weeks, Charles River Laboratories, Wilmington, MA) were anesthetized with isoflurane and oxygen. A 10 mM stock solution of NIR fluorophores was prepared in DMSO, and 25 nmol of each molecule was diluted in BSA-containing saline (5 wt/v %) for intraoperative imaging. After 4 h post-injection, mice were imaged. For each experiment, camera exposure time and image normalization were held constant.

Quantitative Analysis: The fluorescence and background intensity of a region of interest (ROI) over each tissue was quantified using ImageJ software (NIH, Bethesda, MD) version 1.45q. The signal-to-background ratio (SBR) was calculated as SBR=fluorescence/background, where background is the signal intensity of neighboring tissues such as muscle. All NIR fluorescence images for a particular fluorophore were normalized identically for all conditions of an experiment.

### Example 1—Preparation of Exemplified Compounds

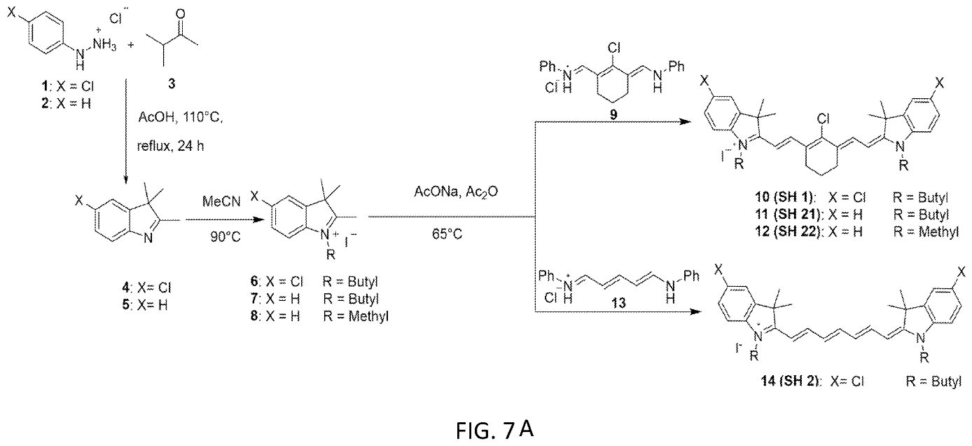

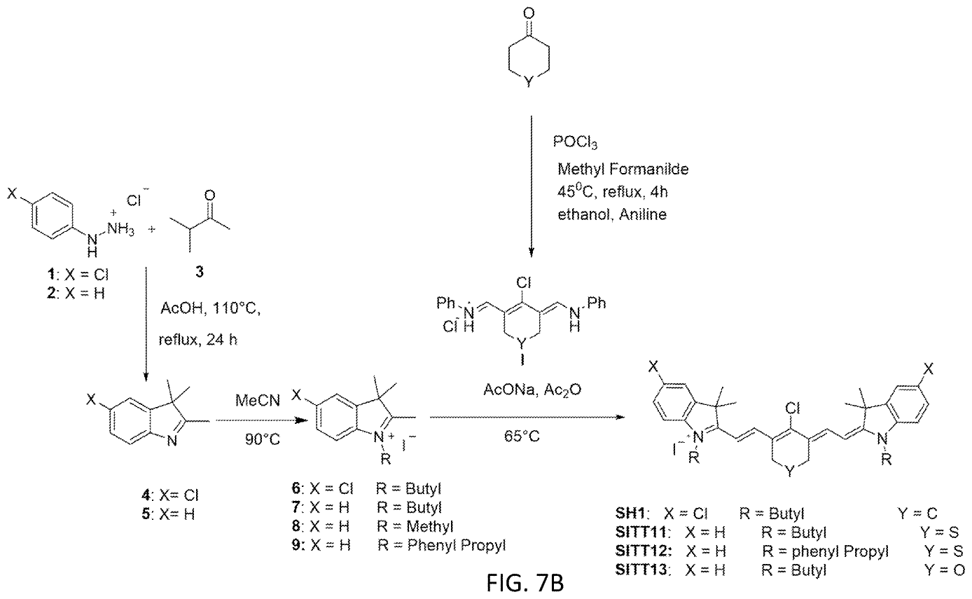

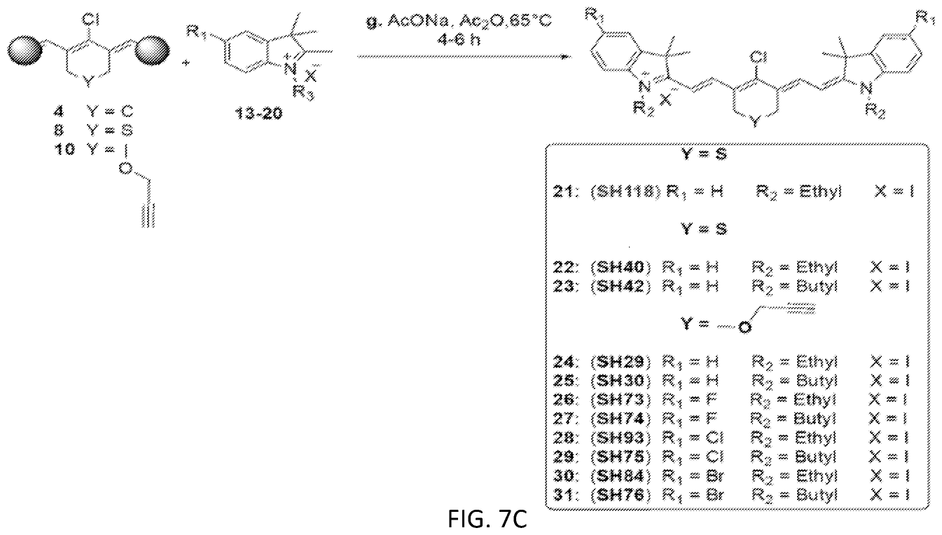

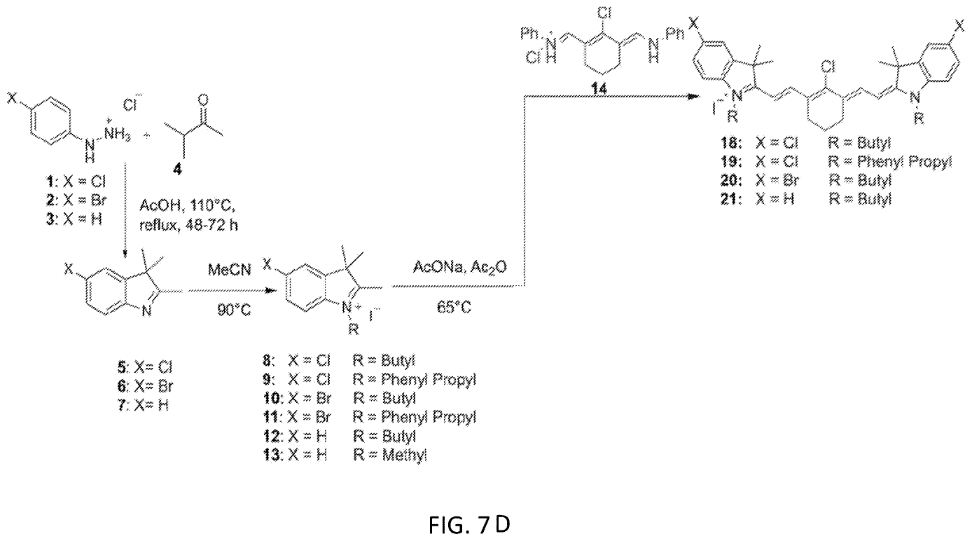

As shown in FIG. 7A, the synthesis of four derivatives of heptamethine cyanine fluorophores 10-12 (SH 1, SH 21, SH 22) and 14 (SH 2) was presented. The synthesis began by the reaction between 4-substituted phenyl hydrazines 1, 2 and 3-methylbutan-2-one 3 to yield 5-substituted indoles 4-5 via the formation of imine intermediates through cyclic rearrangement under acidic condition. Then, various alkyl halides (Iodomethane, 1-Iodobutane) were allowed to react with indoles 4-5 in boiling acetonitrile to furnish the heterocyclic 3H-indolium salts 6-8. The synthesis of heptamethine cyanine derivatives 10-12 (Scheme 1) was achieved by the condensation reaction between Vilsmeier-Haack reagent 9 and individual heterocyclic salt 6-8, in boiling acidic anhydride under basic condition. Another heptamethine cyanine dye 14 was accomplished by the same condensation reaction condition as discussed above between glutaconaldehydedianil hydrochloride 13 and salt 6.

The formation of each dye reaction was monitored by both TLC, and UV-vis spectrometry to ensure the consumption of the starting materials. The crude heptamethine dyes 10-12 and 14 (FIG. 7A) were purified by precipitation using methonal/diethyl ether and column chromatography using 5-10% methanol in DCM as an eluent. The structure of the final heptamethine cyanine derivatives were characterized and confirmed by ¹H NMR, ¹³C NMR, and HR-MS.

General procedure for the preparation of heptamethine cyanine fluorophores 10-12 (SH 1, SH 21, SH 22) and 14 (SH 2): Individual heterocycle indolium salt 6-8 (2 mol equiv.) was added to a clean dried round bottom flask (100 mL) and dissolved in acetic anhydride (5 mL), which then reacted with either Vilsmeier-Haack reagent 9 (1 mol equiv.) or commercially available glutaconaldehydedianil hydrochloride 13 (1 mol equiv.) in the presence of acetic anhydride (5 mL) and sodium acetate (3.5 mol equiv.) to yield the final heptamethine dyes 10-12 and 14 respectively. The reaction mixture was vigorously stirred for 2-8 h at 65° C. and during this time period, the solution gradually turned dark green. The reaction mixture was monitored by Vis/NIR spectrophotometry by analyzing the change of the relative ratios between the expected absorption band (>750 nm) and the starting material absorption peaks (<500 nm) in methanol followed by thin layer chromatography (TLC) in dichloromethane (DCM) and 5% methanol as the eluting solvent. The reaction mixture was allowed to cool to room temperature then the solid of each dye was collected and purified via flash column chromatography using 5% methanol in DCM. The solution of the pure fractions was collected and condensed under reduced pressure to produce dark green solids, which were then dried under vacuum. The final pure products were obtained with fair to very good yields (49%-72%).

### 1-butyl-2-((E)-2-((E)-3-(2-((E)-1-butyl-5-chloro-3,3-dimethylindolin-2-ylidene) ethylidene)-2-chlorocyclohex-1-en-1-yl) vinyl)-5-chloro-3,3-dimethyl-3H-indol-1-ium 10 (SH 1)

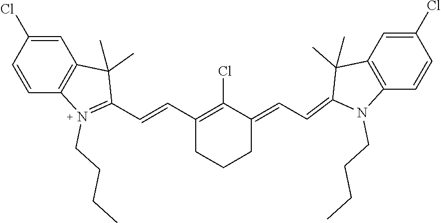

Yield (59%, 0.180 g); mp 156-158° C.; ¹H NMR (400 MHz, CDCl₃): δ ppm 0.9957 (t, J=7.28 Hz, 6H), 1.48 (m, 4H), 1.71 (s, 12H), 1.82 (m, 4H), 1.97 (m, 2H), 2.74 (m, 4H), 4.21 (t, J=7.14, 4H), 6.25 (d, J=14.10, 2H), 7.11 (d, J=8.42, 2H), 7.31 (s, 2H), 7.35 (d, J=8.42, 2H), 8.32 (d, J=14.08, 2H); 13C NMR (100 =MHz, CDCl₃): δ ppm 13.94, 20.36, 20.68, 26.81, 28.11, 29.49, 45.23, 49.33, 102.01, 112.00, 122.81, 128.28, 128.93, 130.88, 140.99, 142.70, 144.16, 150.64, 171.81; λ_{abs}=790 nm in MeOH; HRMS (ESI) Calcd, for [C₃₈H₄₆Cl₃N₂]¹⁺ m/z 637.1456, found m/z 637.1230.

The 12 protons observed at 1.71 ppm chemical shift corresponded to the protons attached to the sp³ carbon of the indolium ring. The triplet at 0.99 ppm corresponded to 6H with a coupling constant of 7.28 Hz, the multiplet signal observed within 1.44-1.50 corresponded to 4H and another multiplet signal within 1.81-1.83 ppm corresponded to 4H, and the triplet at 4.21 ppm with a coupling constant of 7.14 Hz corresponded to 4H. All these signals corresponded to the butyl chain attached to the indolium heterocycle rings. The sp³ protons within the heptamethine rings were observed as multiplets with the chemical shifts within 1.96-1.99 ppm integrating to 2H, and the triplet signal corresponded to 4H with a chemical shift of 2.74 ppm with coupling constant 5.66 Hz. Doublets at 7.11 ppm and 7.35 ppm corresponded to Hb and Hc, respectively, with coupling constant of 8.42 Hz. The singlet signal at 7.32 ppm corresponded to Ha. There were chemical shifts at 6.25 and 8.32 ppm, respectively, with a similar coupling constant of 14.1 Hz. The structure of SH1 produced 19 distinct carbon signals in the ¹³C NMR spectrum. Among those, 8 aliphatic carbon signals were seen below 60 ppm, and the other 11 signals were seen in the aromatic region between 110-180 ppm.

The molecular weight of SH1 was 637.15 therefore the peak found at m/z 637.12 on the spectrum corresponded to the molecular ion via electrospray ionization time-of-flight. The m/z at 635.27, 638.15, 639.15, and 640.17 were due to isotopic clusters arising from Cl, C, and H. In addition, the fragments seen at m/z 692.26 were from the ESI matrix. When fragmented, the structure of SH1 lost the two-terminal chlorine atoms and ethyl groups from both N atoms to yield a new fragment at m/z 512.16. Loss of one of the indolium rings from the previous fragment yielded a new fragment at m/z 352.93. Loss of one chlorine atom from the meso position and conjugated diene from the previous fragment yielded the new fragment at m/z 280.43-2.35=278.08. Loss of an ethyl group from the previous fragment produced a new fragment at m/z 251.37.

### 1-butyl-2-((1E,3E,5E)-7-((E)-1-butyl-5-chloro-3,3-dimethylindolin-2-ylidene) hepta-1,3,5-trien-1-yl)-5-chloro-3,3-dimethyl-3H-indol-1-ium iodide 14 (SH 2)

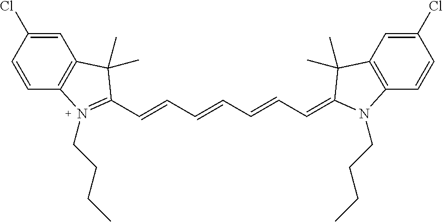

Yield (51%, 0.140 g); mp 162-164° C.; ¹H NMR (400 MHz, DMSO-d₆): δ ppm 0.91 (t, J=7.30 Hz, 6H), 1.37 (m, 4H), 1.63 (s, 16H), 4.05 (t, J=7.03, 4H), 6.39 (d, J=13.69, 2H), 6.57 (t, J=12.59, 2H), 7.41 (dd, J=10.92, 1.80, 4H), 7.88 (m, 4H); 13C NMR (100 MHz, CDCl₃): δ ppm 13.93, 20.33, 28.08, 29.46, 45.58, 49.15, 104.30, 111.33, 122.82, 127.14, 128.66, 130.32, 140.95, 142.74, 151.71, 154.20, 157.55, 170.95; λabs=755 nm in MeOH; HRMS (ESI) Calcd, for [C₃₅H₄₃Cl₂N₂]¹⁺ m/z 562.6456, found m/z 561.2798.

The SH2 fluorophore had similar indolium salts like SH1 connected by an open heptamethine chain, where SH1 had a cyclic ring and chlorine atom in the meso position of the structure. Overall, SH 2 produced 43 proton signals. Among those, thirteen downfield signals were from aromatic carbon (labeled Ha, Hb, and Hc) and polymethine bridge. SH2 fluorophore showed a characteristic dye signal as a doublet at 6.39 ppm corresponding to 2H having a coupling constant of 13.69 Hz and two triplets at 6.57 corresponding to 2H and another one at 7.89 ppm corresponding to 3H with a similar coupling constant of 12.72 Hz. The other 6 aromatic proton signals from SH2 came as a doublet of a doublets at 7.41 ppm corresponding to 4H having coupling constants 10.92 Hz and 1.80 Hz and as a singlet at 7.75 ppm corresponding to 2H. The structure of SH2 produced 18 distinct carbon signals in the ¹³C NMR spectrum. Among those, 6 aliphatic carbons were present below 60 ppm and the remaining 12 signals were found in the aromatic region between 110-180 ppm.

The molecular weight of SH2 was 562.64, therefore the peak found in electrospray ionization time-of-flight at m/z 562.64 was the molecular ion. Due to the isotopic mass of Cl, C, and H, molecular ion peaks found at m/z 564.18, 565.19 and 566.21 were present. When fragmented, the structure of SH2 lost one chlorine atom from one terminal and produced a new fragment at m/z 527.17. Loss of the methyl groups on both sides of the alkyl chains from the previous fragment gave a new fragment at m/z 500.15. Loss of the butyl and propyl groups from the alkyl chain of the previous fragment formed a new fragment at m/z 430.01. Loss of the indole ring from one side, polymethine bridge, and methyl group from the previous fragment gave a new fragment at m/z at 159.23.

### 1-butyl-2-((E)-2-((E)-3-(2-((E)-1-butyl-3,3-dimethylindolin-2-ylidene)ethylidene)-2-chlorocyclohex-1-en-1-yl) vinyl)-3,3-dimethyl-3H-indol-1-ium iodide 11 (SH 21)

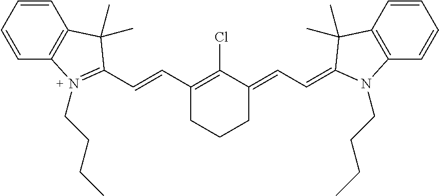

Yield (72%, 0.220 g); mp >200° C.; ¹H NMR (400 MHz, CDCl₃): δ ppm 0.1.01 (t, J=7.26 Hz, 6H), 1.50 (m, 4H), 1.72 (s, 12H), 1.84 (m, 4H), 2.07 (m, 2H), 2.73 (m, 4H), 4.20 (t, J=7.10, 4H), 6.21 (d, J=14.08, 2H), 7.18 (d, J=8.08, 2H), 7.26 (dd, J=18.96, 7.44, 2H), 7.39 (d, J=5.20, 4H), 8.35 (d, J=14.08, 2H); ¹³C NMR (100 MHz, CDCl₃): δ ppm 13.94, 20.38, 20.73, 26.70, 28.16, 29.52, 44.89, 49.36, 101.33, 111.00, 122.30, 125.35, 127.29, 128.82, 141.06, 142.44, 144.30, 150.50, 172.37; λ_{abs}=780 nm in MeOH.

### 5-chloro-2-((E)-2-((E)-2-chloro-3-(2-((E)-5-chloro-1,3,3-trimethylindolin-2-ylidene)ethylidene) cyclohex-1-en-1-yl) vinyl)-1,3,3-trimethyl-3H-indol-1-ium 12 (SH 22

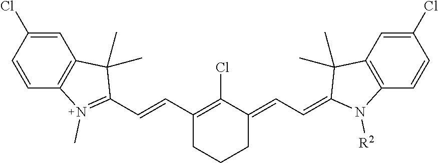

Yield (49%, 0.149 g); mp>200° C.; ¹H NMR (400 MHz, DMSO-d₆): δ ppm 0.77 (m, 2H), 0.99 (m, 2H), 1.34 (s, 6H), 1.58 (s, 6H), 2.25 (m, 2H), 2.39 (s, 3H), 2.9 (s, 3H), 5.24 (d, J=12.65 Hz, 1H), 5.60 (d, J=15.73 Hz, 1H) 6.26 (d, J=7.64 Hz, 1H), 6.59 (d, J=8.28 Hz, 1H), 6.6 (s, 2H), 6.98 (m, 3H), 7.13 (s, 1H); 13C NMR (100 MHz, DMSO-d₆): δ ppm 20.07, 21.63, 23.72, 26.30, 27.47, 27.63, 27.96, 29.19, 29.59, 45.67, 48.99, 93.73, 99.91, 108.00, 108.36, 121.58, 122.19, 122.52, 123.57, 123.98, 125.24, 127.10, 127.90, 128.71, 129.95, 131.184, 132.38, 140.41, 140.85, 144.24, 148.48, 157.65; λabs=784 nm in MeOH.

The SH21 and SH22 fluorophores had similar structures to SH1. The difference between SH21 and SH1 was their alkyl chain length connected to N-atoms in the indolium rings. In SH1, the butyl group was connected to Cl substituted indolium salt, but the methyl group was connected to SH22. On the other hand, the only difference was SH21 fluorophore has no substituted chlorine atoms in the aromatic ring, which was the reason SH21 produced 2 more proton signals than SH1.

Overall, SH21 fluorophore produced 48 proton signals and SH22 produced 34 proton signals. Among those, SH21 generated 12 proton signals and SH22 generated 10 proton signals in the downfield region from aromatic carbon and polymethine bridge. SH21 fluorophore showed characteristic dye signal doublets at 6.21 ppm and 8.35 ppm corresponding to a total of 4 protons with the same coupling constant 14.08 Hz. The other 8 aromatic proton signals from SH21 were seen in the downfield region, which showed a doublet corresponding to 2H at 7.18 ppm with coupling constant 8.08 Hz, doublet of doublets corresponding to 2H at 7.26 ppm with a coupling constants 18.96 Hz, 7.44, 2H, and a doublet corresponding to 4H at 7.39 ppm with a coupling constant 14.08 Hz. SH22 fluorophore also showed characteristics 4 proton signal (doublets) in the downfield region at 5.24 ppm, 5.60 ppm, 6.26 ppm, and 6.59 ppm corresponding to each single proton in the polymethine bridge having coupling constants of 12.65 Hz, 15.73 Hz, 7.64 Hz, and 8.28 Hz respectively. The other 6 aromatic protons were found as a singlet at 6.6 ppm and 7.13 ppm corresponding to 2H and 1H, respectively, and multiples at 6.96-7.04 ppm corresponding to 3H.

The structure of SH21 produced 19 distinct carbon signals in the 13C NMR spectrum, which was similar to SH1. Among those, 8 aliphatic carbons were shown below 60 ppm and the remaining 11 signals were found in the aromatic region between 110-180 ppm. The SH22 fluorophore also produced 8 aliphatic distinct carbon signals below 60 ppm but this structure showed all the conjugated carbon signals in 90-160 ppm.

### Example 2—Preparation of Exemplified Compounds

As shown in FIG. 7B, the synthesis of heptamethine cyanines with a rigid cyclohexenyl ring containing sulfur and oxygen in the polymethine backbone was accomplished. Briefly, individual heterocycle indolium salt 6-9 (2 mol equiv.) was added to a clean dried round bottom flask (100 mL) and dissolved in acetic anhydride (5 mL), which then reacted with either Vilsmeier-Haack reagent I (1 mol equiv.) in the presence of acetic anhydride (5 mL) and sodium acetate (3.5 mol equiv.) to yield the final SH1 adn SITT 11-13. The reaction mixture was vigorously stirred for 2-8 h at 65° C. and during this time period, the solution gradually turned dark green. The reaction mixture was monitored by Vis/NIR spectrophotometry by analyzing the change of the relative ratios between the expected absorption band (>750 nm) and the starting material absorption peaks (<500 nm) in methanol followed by thin layer chromatography (TLC) in dichloromethane (DCM) and 5% methanol as the eluting solvent. The reaction mixture was allowed to cool to room temperature then the solid of each dye was collected and purified via flash column chromatography using 5% methanol in DCM. The solution of the pure fractions was collected and condensed under reduced pressure to produce dark green solids, which were then dried under vacuum. The final pure products were obtained with fair to very good yields (49%-72%).

### 1-butyl-2-((E)-2-((E)-5-(2-((E)-1-butyl-5-chloro-3,3-dimethylindolin-2-ylidene)ethylidene)-4-chloro-5,6-dihydro-2H-thiopyran-3-yl) vinyl)-5-chloro-3,3-dimethyl-3H-indol-1-ium iodide. (SITT11)

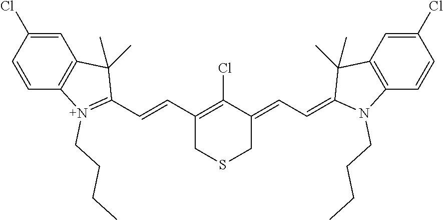

Yield (53%, 0.183 g); mp >200° C.; ¹H NMR (400 MHz, CDCl₃)): δ ppm 0.99 (t, J=7.21, 6H), 1.49 (m, 4H), 1.72 (s, 12H), 1.82 (m, 4H), 3.83 (s, 4H), 4.29 (t, J=6.88, 4H), 6.31 (d, J=14.29, 2H), 7.19 (d, J=8.37, 2H), 7.35 (s, 2H), 7.38 (d, J=8.52, 2H), 8.37 (d, J=14.26, 2H); 13C NMR (100 MHz, CDCl₃): δ ppm 13.96, 20.36, 28.08, 28.48, 29.59, 45.55, 49.58, 101.98, 112.45, 122.86, 124.64, 129.04, 131.31, 140.79, 142.79, 144.39, 150.80 172.94.

### 5-chloro-2-((E)-2-((E)-4-chloro-5-(2-((E)-5-chloro-3,3-dimethyl-1-(3-phenylpropyl) indolin-2-ylidene)ethylidene)-5,6-dihydro-2H-thiopyran-3-yl) vinyl)-3,3-dimethyl-1-(3-phenylpropyl)-3H-indol-1-ium bromide (SITT12)

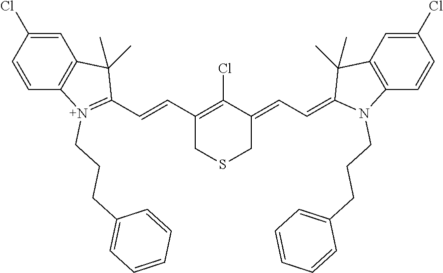

5-chloro-2-((E)-2-((E)-4-chloro-5-(2-((E)-5-chloro-3,3-dimethyl-1-(3-phenylpropyl) indolin-2-ylidene)ethylidene)-5,6-dihydro-2H-thiopyran-3-yl) vinyl)-3,3-dimethyl-1-(3-phenylpropyl)-3H-indol-1-ium bromide (12). Yield (59%, 0.208 g); mp 194-196° C.; ¹H NMR (400 MHz, CDCl₃): δ ppm 1.67 (s, 12H), 2.10 (br, 4H), 2.89 (br, 4H), 3.49 (s, 4H), 4.28 (br, 4H), 6.01 (d, J=14.19, 2H), 7.10 (d, J=8.25, 2H), 7.28-7.35 (br, 14H), 8.28 (d, J=14.20, 2H); 13C NMR (100 MHz, CDCl₃): δ ppm 28.00, 28.13, 32.51, 44.10, 49.48, 101.74, 112.49, 122.77, 124.80, 126.68, 128.69, 128.83, 129.13, 131.33, 140.23, 140.55, 142.69, 144.31, 150.66, 172.58.

### 1-butyl-2-((E)-2-((E)-5-(2-((E)-1-butyl-5-chloro-3,3-dimethylindolin-2-ylidene)ethylidene)-4-chloro-5,6-dihydro-2H-pyran-3-yl) vinyl)-5-chloro-3,3-dimethyl-3H-indol-1-ium iodide (SITT13)

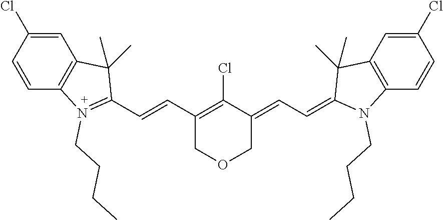

Yield (50%); mp >200° C.; ¹H NMR (400 MHz, CDCl₃): δ ppm 0.99 (t, J=7.21, 6H), 1.49 (m, 4H), 1.72 (s, 12H), 1.82 (m, 4H), 3.83 (s, 4H), 4.29 (t, J=6.88, 4H), 6.31 (d, J=14.29, 2H), 7.19 (d, J=8.37, 2H), 7.35 (s, 2H), 7.38 (d, J=8.52, 2H), 8.37 (d, J=14.26, 2H); 13C NMR (100 MHz, CDCl₃): δ ppm 13.96, 20.36, 28.08, 28.48, 29.59, 45.55, 49.58, 101.98, 112.45, 122.86, 124.64, 129.04, 131.31, 140.79, 142.79, 144.39, 150.80 172.94.

### Example 3—Preparation of Exemplified Compounds

As shown in FIG. 7C, the synthesis of heptamethine cyanines with a rigid cyclohexenyl ring containing sulfur in the polymethine backbone or modified with propargyl alcohol was accomplished using a similar procedure as Examples 1 and 2. Heterocycle indolium salt 13-20 (2 mol equiv.) was dissolved in acetic anhydride, which then reacted with Vilsmeier-Haack reagent 4, 8, or 10 (1 mol equiv.) in the presence of acetic anhydride and sodium acetate to yield SH29-30, SH40, SH42, SH73-76, SH84, SH93, and SH118. The reaction mixture was stirred for 4-6 h at 65° C.

### Example 4—Preparation of Exemplified Compounds

As shown in FIG. 7D, hepthamethine cyanines with a rigid cylohexenyl ring were synthesized. The synthesis started with the formation of indole rings 5-7 through Fischer indole synthesis. A starting material 4-substituted phenyl hydrazine derivative 1, 2, or 3 was refluxed under an acidic condition with 3-methylbutan-2-one 4 for cyclization. Each cyclization reaction proceeded through the formation of imine derivatives and refluxed for 48-72 h at 110° C. to complete the reaction. After cooling the reaction mixture to room temperature, substituted indole rings 5-7 were achieved as a brown oil by extracting the reaction mixture in DCM and NaHCO₃(aq). In the next step, heterocyclic 3H-indolium salts 8-13 were synthesized, where N-alkylation to the cyclic indole rings was obtained by refluxing with various alkyl halides (Iodomethane, 1-Iodobutane, 1-bromo-3-phenyl propane) in boiling acetonitrile. The heterocyclic salts 8-13 were purified by performing several recrystallizations in DCM: ether, acetone: ether, EtOAc/ether, and MeOH/ether. After purification, each of these individual salts was allowed to be condensed with various linkers 14, 15, 16, or 17 separately under basic conditions to form the final desired products. For example, heptamethine cyanine derivatives containing cyclohexenyl rings 18-21 were achieved by the condensation reaction between Vilsmeier-Haack reagent 14 and individual heterocyclic salt 8, 9, 10, 12, or 13. Finally, the crude products were purified by flash column chromatography in 5% methanol in DCM and dried under vacuum to obtain green crystals for the heptamethine cyanine fluorophores.

Flurophore 18 in FIG. 7D is the same compound as SH1 in FIG. 7A.

### 5-Chloro-2-((E)-2-((E)-2-chloro-3-(2-((E)-5-chloro-3,3-dimethyl-1-(3-phenylpropyl) indolin-2-ylidene)ethylidene)cyclohex-1-en-1-yl)vinyl)-3,3-dimethyl-1-(3-phenylpropyl)-3H-indol-1-ium Bromide (19)

Yield (49%, 0.15 g); mp 181-183° C.; ¹H NMR (400 MHz, CDCl₃): δ ppm 1.69 (s, 12H), 1.93 (br, 2H), 2.17-2.18 (m, 4H), 2.57 (br, 4H), 2.87-2.90 (m, 4H), 4.27 (t, J=6.88 Hz, 4H), 6.26 (d, J=14.1 Hz, 2H), 6.99 (d, J=8.32 Hz, 2H), 7.33 (m, 14H), 8.25 (d, J=14.1 Hz, 2H), ¹³C NMR (100 MHz, CDCl₃): δ ppm 1.0, 15.3, 20.6, 28.0, 28.5, 32.7, 44.0, 49.3, 65.9, 101.9, 112.0, 122.7, 128.4, 128.6, 128.7, 129.0, 130.9, 140.3, 142.6, 144.1, 150.5, 171.5; λ_{abs}=790 nm in MeOH.

### 5-Bromo-2-((E)-2-((E)-3-(2-((E)-5-bromo-1-butyl-3,3-dimethylindolin-2-ylidene)ethylidene)-2-chlorocyclohex-1-en-1-yl) vinyl)-1-butyl-3,3-dimethyl-3H-indol-1-ium Iodide (20)

Yield (54%, 0.16 g); mp 152-154° C.; ¹H NMR (400 MHz, DMSO-d₆): δ ppm 0.95 (t, J=7.34 Hz, 6H), 1.41-1.47 (m, 4H), 1.72 (s, 16H), 1.86-1.90 (m, 2H), 2.71-2.74 (m, 4H), 4.19 (t, J=7.23 Hz, 4H), 6.31 (d, J=14.1 Hz, 2H), 7.40 (d, J=8.51 Hz, 2H), 7.60 (dd, J=8.46 Hz, 1.71 Hz, 2H), 7.87 (d, J=1.67 Hz, 2H), 8.27 (d, J=14.1 Hz, 2H), ¹³C NMR (100 MHz, DMSO-d₆): δ ppm 14.2, 19.9, 20.8, 26.3, 27.8, 29.6, 44.3, 49.6, 102.5, 113.9, 118.0, 126.3, 127.4, 131.9, 141.9, 143.6, 143.9, 148.9, 172.3; Aabs=795 nm in MeOH.

### 1-Butyl-2-((E)-2-((E)-3-(2-((E)-1-butyl-3,3-dimethylindolin-2-ylidene)ethylidene)-2-chlorocyclohex-1-en-1-yl) vinyl)-3,3-dimethyl-3H-indol-1-ium Iodide (21)

Yield (72%, 0.22 g); mp >200° C.; ¹H NMR (400 MHz, CDCl₃): δ ppm 1.01 (t, J=7.26 Hz, 6H), 1.49-1.53 (m, 4H), 1.72 (s, 12H), 1.85-1.86 (m, 4H), 2.08 (br, 2H), 2.71-2.74 (m, 4H), 4.20 (t, J=7.10 Hz, 4H), 6.21 (d, J=14.1 Hz, 2H), 7.18 (d, J=8.08 Hz, 2H), 7.26 (dd, J=8.96 Hz, 7.44 Hz, 2H), 7.39 (d, J=5.20 Hz, 4H), 8.35 (d, J=14.1 Hz, 2H); ¹³C NMR (100 MHz, CDCl₃): δ ppm 13.9, 20.4, 20.7, 26.7, 28.2, 29.5, 44.9, 49.4, 101.3, 111.0, 122.3, 125.4, 127.3, 128.8, 141.1, 142.4, 144.3, 150.5, 172.4; Aabs=780 nm in MeOH.

### Example 5—Cancer Imaging Studies with Exemplified Compounds

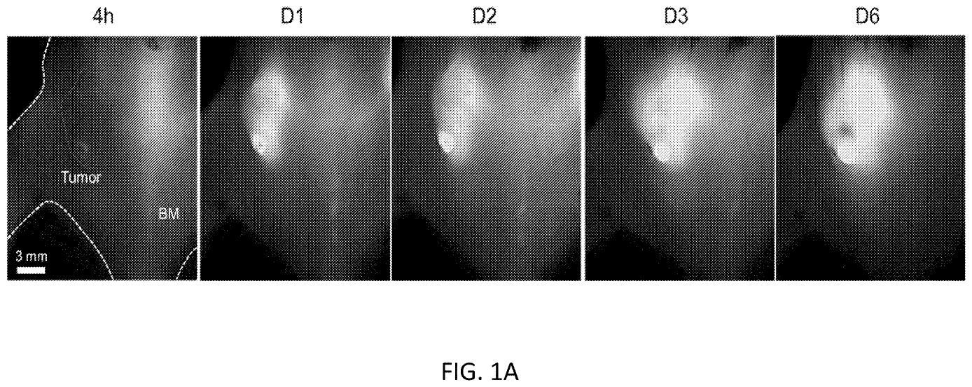

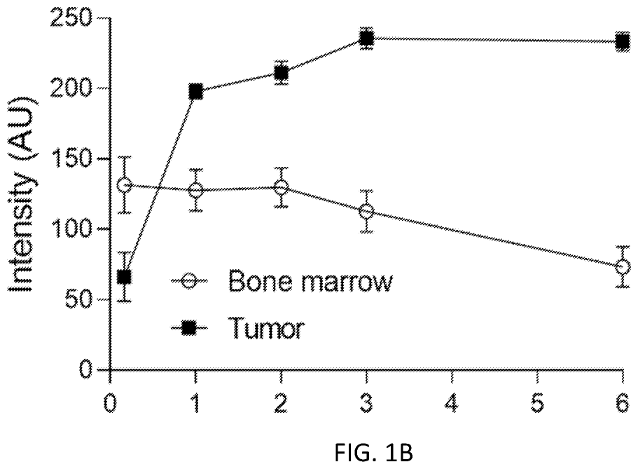

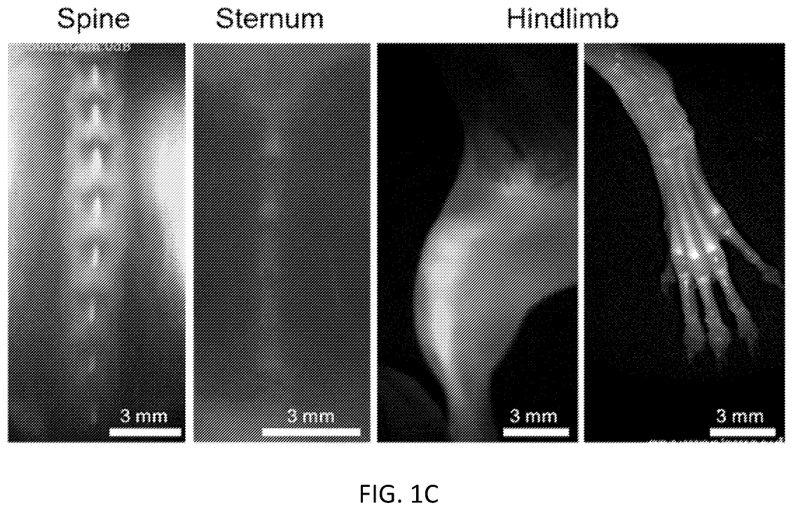

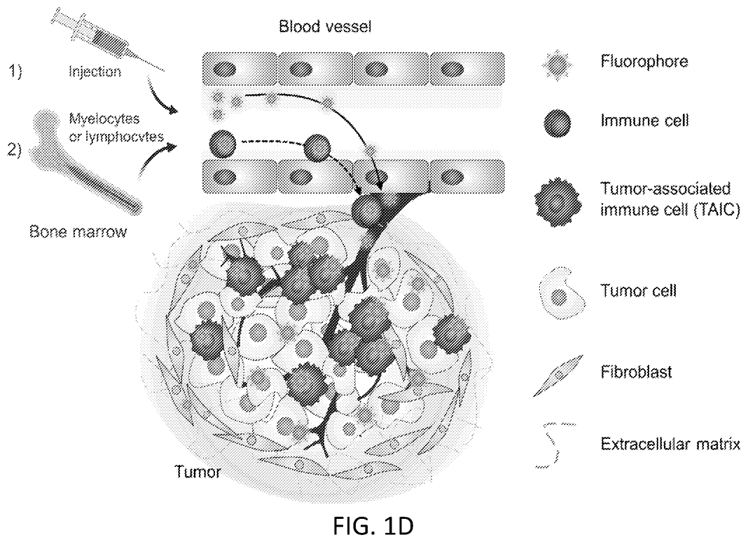

Indocyanine-based fluorophores lacking targeting moieties have occasionally shown tumor targetability, called structure-inherent tumor targeting (SITT), which have a native cancer-targeting property resulting in no need for further chemical conjugation of targeting moieties. Without wishing to be bound by theory, the targeting mechanisms are either membrane transporter mediated (i.e., organic anion transporters; OATP) and/or via the albumin mediated enhanced permeability and retention (EPR) effect. If the dye can directly target tumor cells, signals should show up during the very first circulation in the bloodstream followed by retention in the tumor cells while background tissue signal should gradually decrease, resulting in a high signal-to-background ratio (SBR). However, upon intravenous injection of the TAIC targeted fluorophore SH1 into tumor-bearing mice, it was observed that signals in the cancerous region did not appear at early time points (even until 24 h post-injection), but rather abruptly showed up hours later (FIGS. 1A-D). This unexpected observation prompted the fundamental question of whether there are other possible mechanisms for SITT. Interestingly, it was observed that a portion of fluorophores accumulated in immune cells in bone marrow, followed by infiltration of those bone marrow-derived immune cells to the cancerous region, which resulted in a fluorescence signal intensity boost over time and a notably improved SBR (FIGS. 1A and 1B). In addition, the bone marrow in the spine, sternum, and hindlimb were clearly seen with non-invasive NIR-II fluorescence imaging at 24 h post-injection (FIG. 1C), owing to the NIR-II imaging capability of deeper penetration depth. Without wishing to be bound by theory, as depicted in FIG. 1D, it is believed that SH1 has two different tumor-targeting mechanisms. Once the fluorophore is injected, a portion of it can directly reach the cancerous region by systemic circulation and can be taken up by tumor cells as well as TRICs over time up to 24 h (the first mechanism in FIG. 1D). In addition to this mode of action, the fluorophore can be internalized by immune cells in the bone marrow followed by infiltration of those bone marrow-derived immune cells to the cancerous region (the second mechanism in FIG. 1D). This simultaneous tumor-targeting mechanism of the SH1 fluorophore causes the signal intensity in subcutaneous tumors to gradually increase over time.

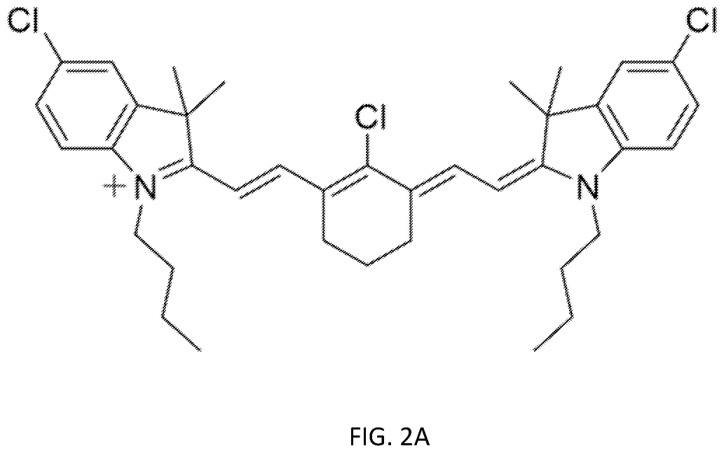

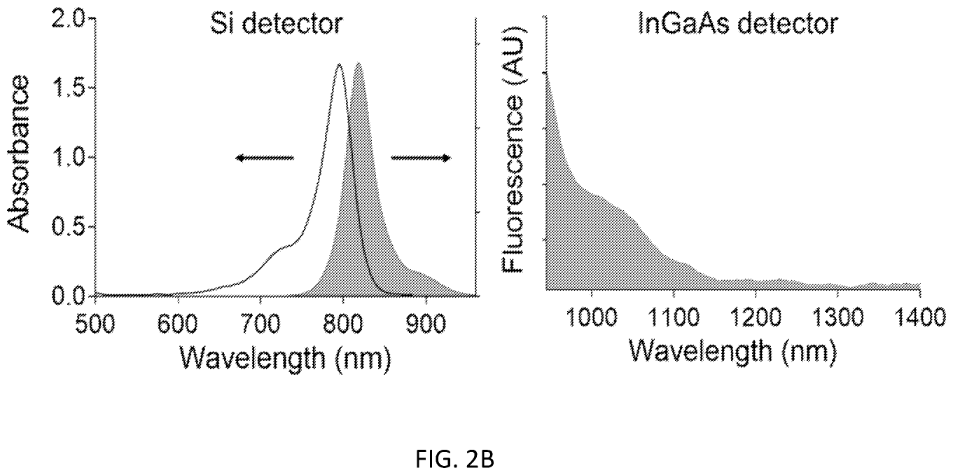

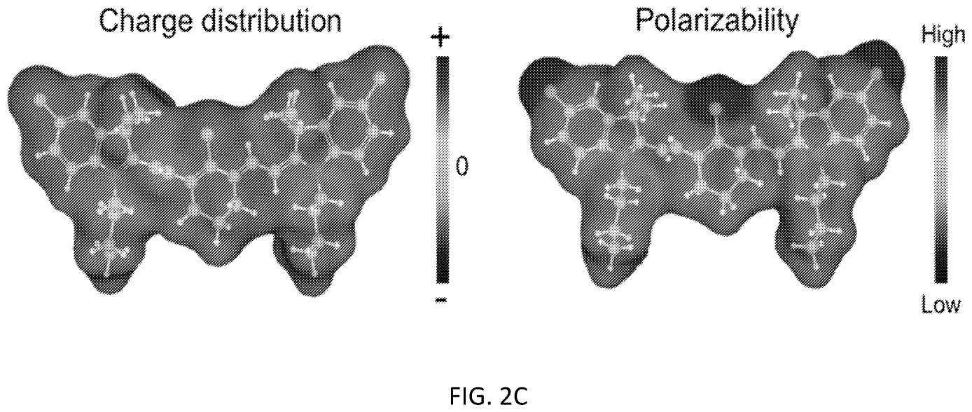

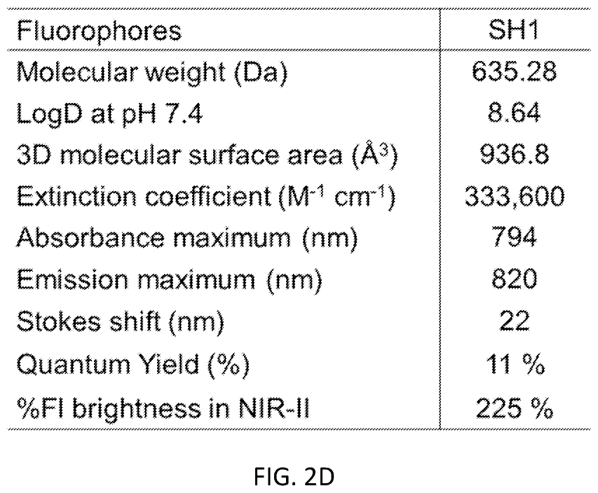

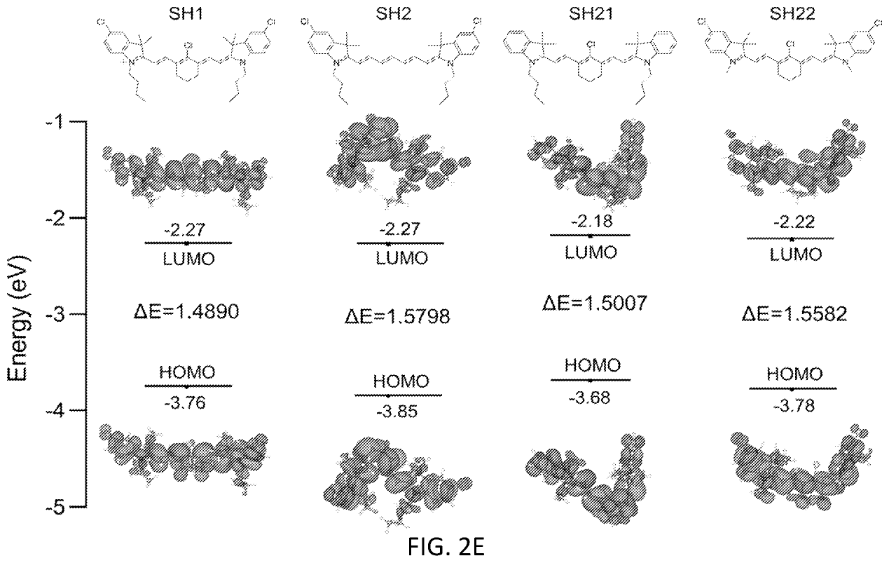

Based on in vivo screening results in the cyanine-based fluorophore library, SH1 was designed, which also has NIR-II imaging capabilities, as shown in FIG. 2A. SH1 was synthesized by condensation reaction between heptamethine core 9 and heterocycle indolium salt 6, resulting in SH1 possessing two butyl chains and two chlorides on both sides of the indolium rings as well as a chloride at the meso position on the heptamethine core (FIG. 7A). The characterization results from ¹H and ¹³C NMR spectra and electrospray ionization time-of-flight (ESI-TOF) mass data were consistent with the proposed structure.

Next, the optical and physicochemical properties such as absorbance and fluorescence spectra, charge distributions, and polarizability of SH1 were investigated (FIGS. 2B-2D). SH1 showed strong absorbance with an extinction coefficient of 333,600 M⁻¹ cm⁻¹, fluorescence emissions in the longer wavelengths of NIR-I (emission maximum is 820 nm) and NIR-II (tail emission up to ˜1250 nm is an important factor in determining the NIR-II capacity). The observations support the notion that NIR-I heptamethine indocyanine dye can be repurposed for NIR-II imaging. SH1 exhibited higher quantum yield in 5 w/v % BSA saline (11%) compared with commercially available NIR-II dyes IR-E1050 and IR26 (0.2-2% and 0.05-0.5%, respectively). SH1 also had higher fluorescence brightness (225%) in the NIR-II region compared with ICG, the only FDA-approved heptamethine NIR fluorophore for NIR-II imaging.

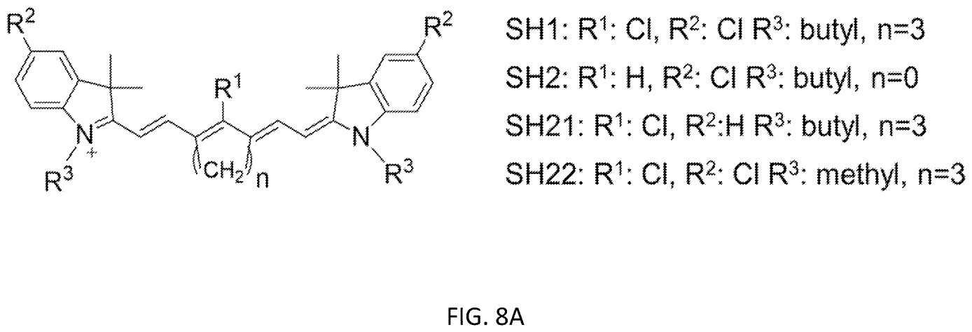

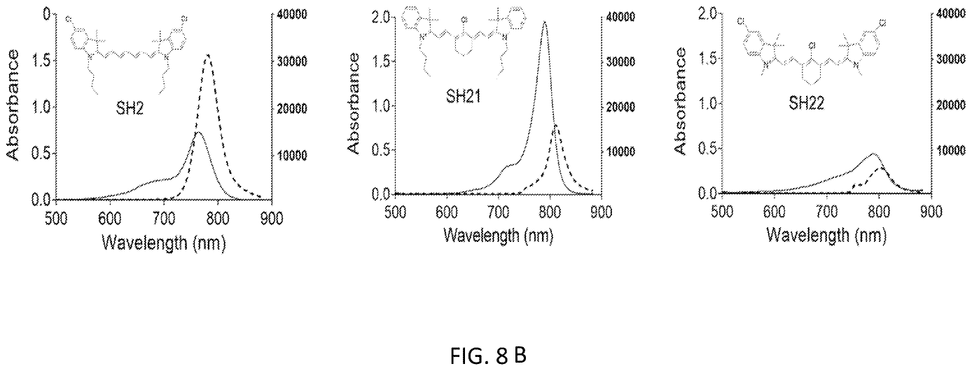

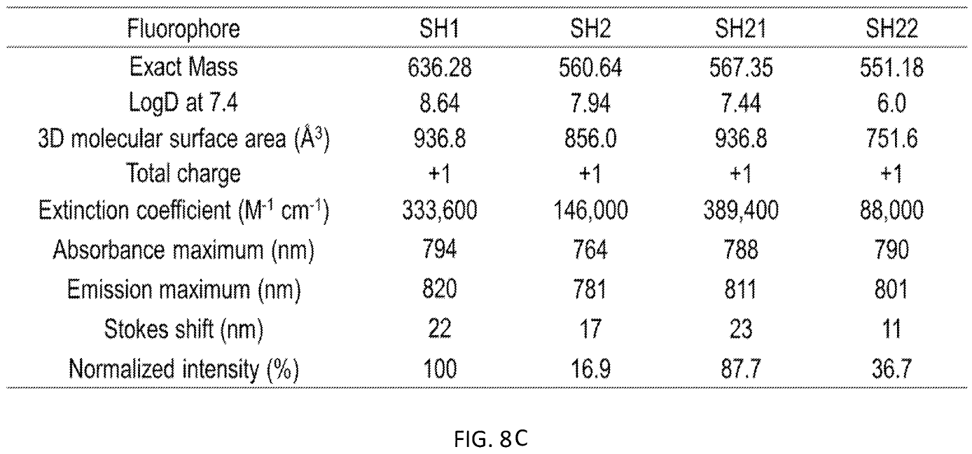

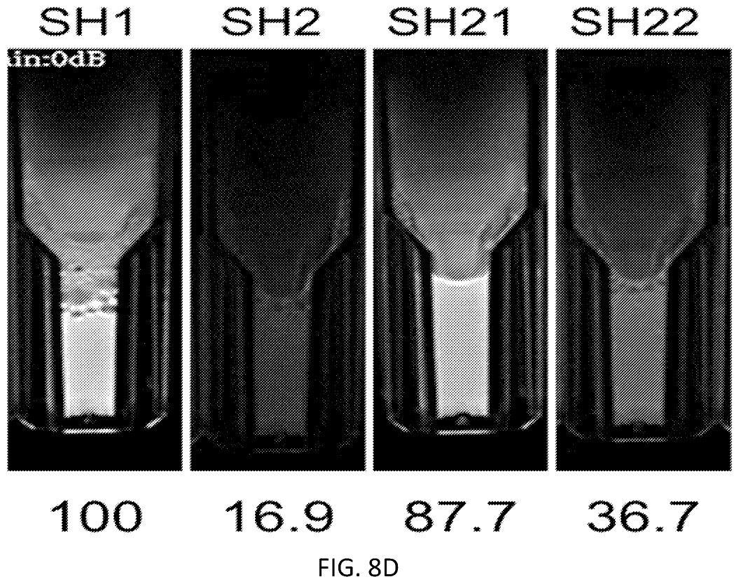

Three more SH derivatives were further synthesized based on the heptamethine core (FIG. 7A) and were compared in terms of the NIR-II imaging capability and tumor targetability. Although all four SH compounds showed similar optical properties, SH1 showed the most dramatic red-shifted absorbance and fluorescence emission (FIG. 8). To understand these fluorophores, density functional theory (DFT) calculations were performed to examine their geometric and fluorescence properties (FIG. 2E) using Gaussian 16 software. It was found that the cyclohexene ring in SH1 gave rigidity to the conjugated alkene framework and prevented rotation along the middle bond, which made it more favorable in producing an asymmetric configuration compared to SH2, and resulted in a lower molecular energy gap between the HOMOs and LUMOs. In addition, the butyl chains in SH1, which were more electron-donating than the methyl groups in SH22, which caused SH1 to have a lower molecular energy gap than SH22.

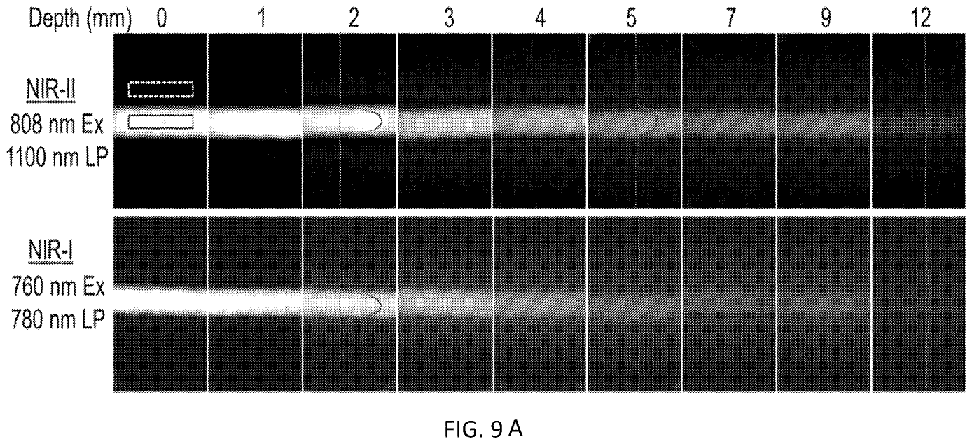

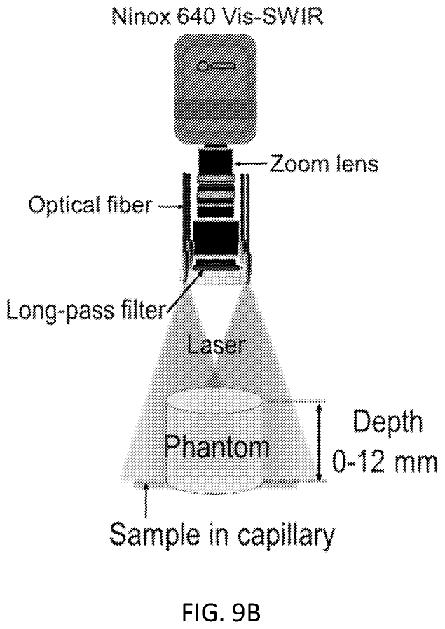

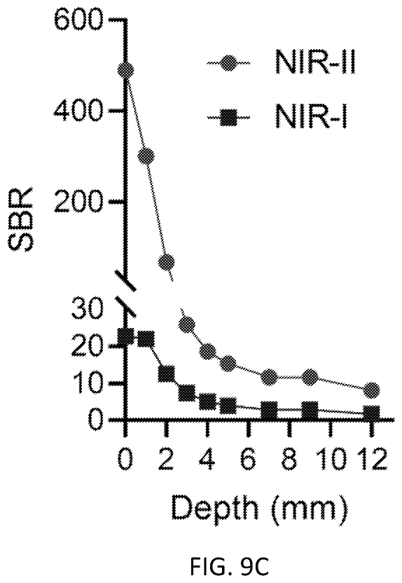

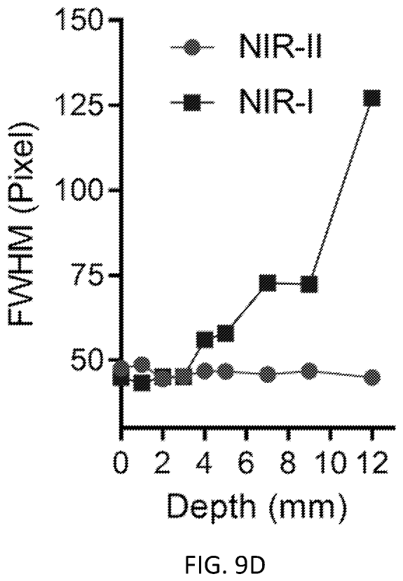

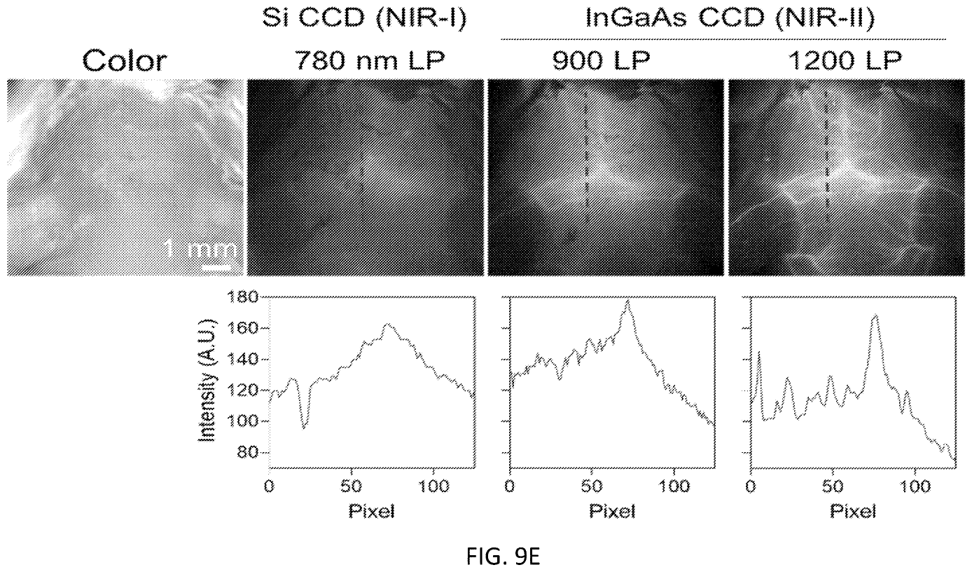

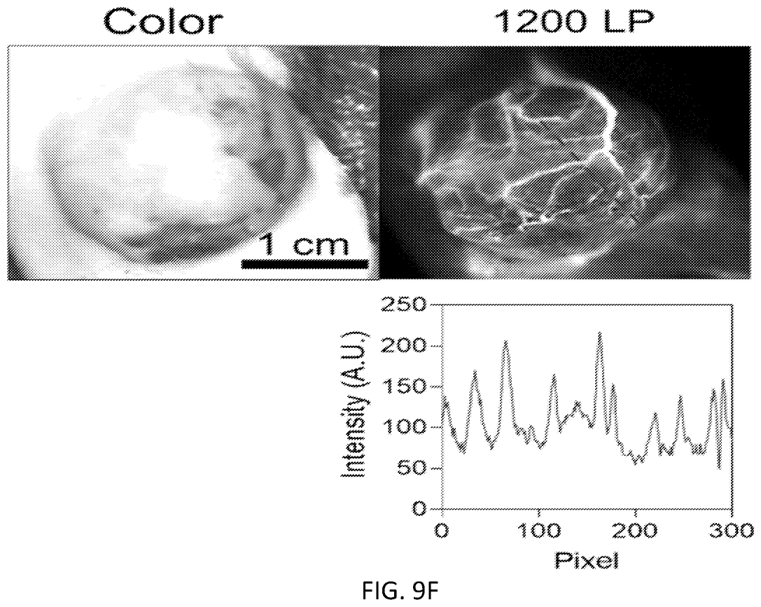

To demonstrate the improved fluorescence imaging in the NIR-II region, NIR-I (>780 nm, using KFLARE system) and NIR-II (>1100 nm, using Ninox InGaAs camera) images of skin phantoms at various depths (0-12 mm) were compared, as shown in FIGS. 9A and 9B. Based on the data, NIR-II imaging had deeper optical penetration with clear resolution and high contrast. SBR and full width at half maximum (FWHM) profiles of capillaries at increasing phantom tissue depths were shown in FIGS. 9C and 9D. The result showed that NIR-II images had 4.8 times higher SBR and 2.8 times narrower FWHM at 12 mm in depth compared to NIR-I, which can be attributed to the reduced scattering at the longer emission wavelength. Furthermore, in vivo brain imaging was performed (FIG. 9E). SH1 (100 μL, 500 μM in 5% BSA saline) was injected into a shaved CD-1 mouse and cerebral vessels were imaged with different imaging settings such as NIR-I with a 780 nm long pass (LP) filter and NIR-II with either 900 nm LP or 1200 nm LP filters. With the 1200 nm LP filter, it was clearly shown that using a longer wavelength greatly attenuated the tissue scattering and autofluorescence. Similarly, blood vessel networks in the tumor were able to be visualized (FIG. 9F).

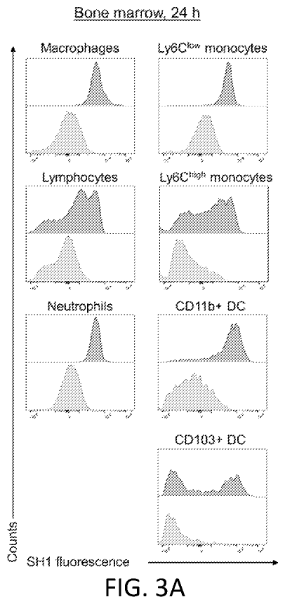

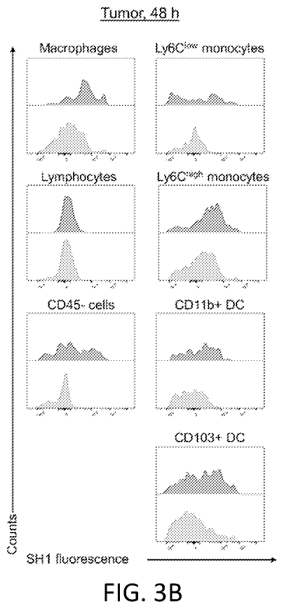

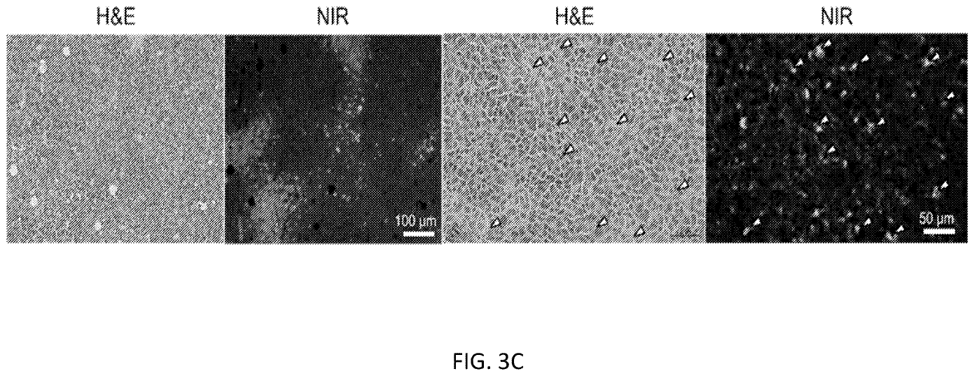

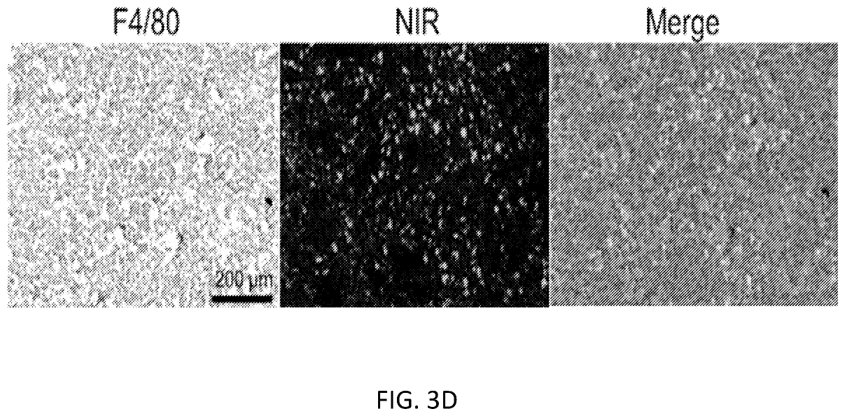

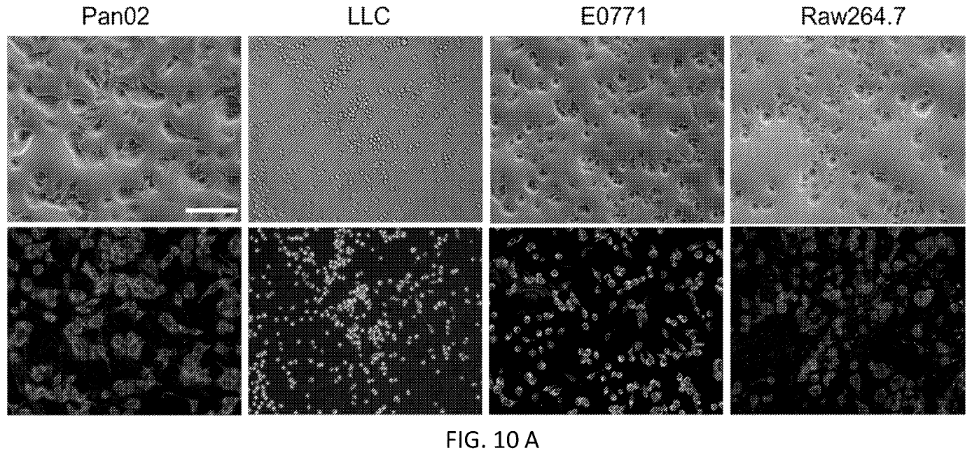

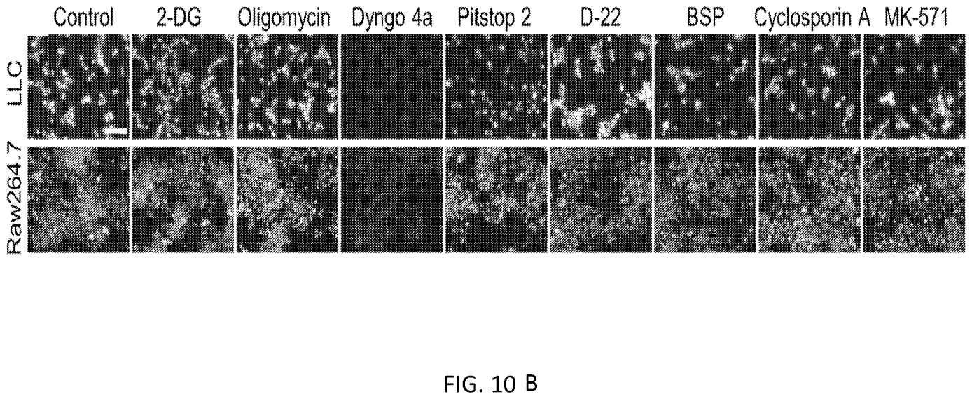

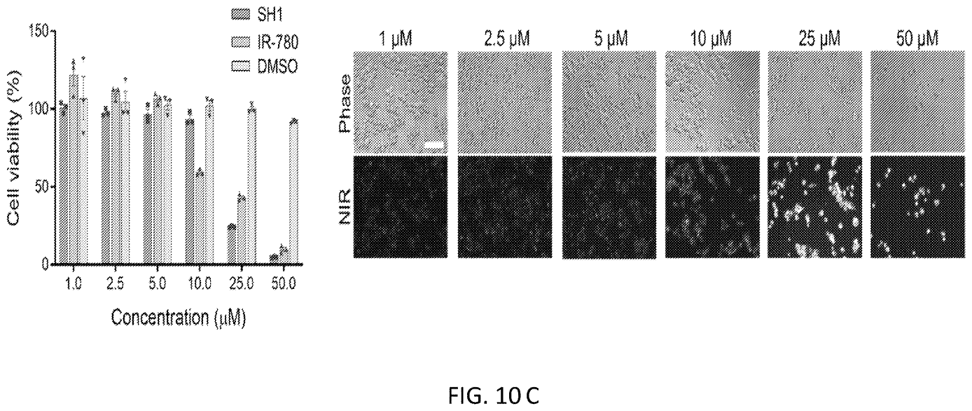

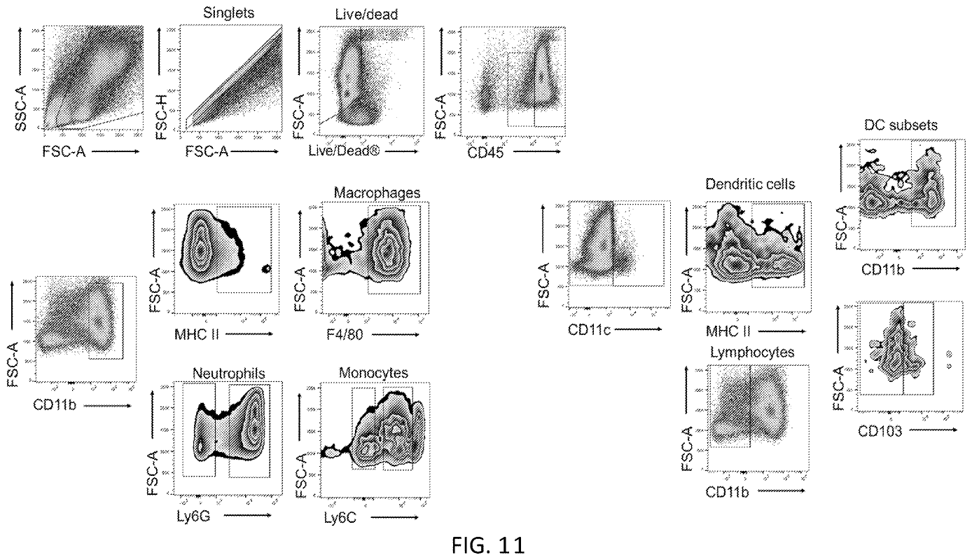

Prior to performing intraoperative tumor targeting with SH1, an in vitro inhibition test was performed to confirm whether SH1 cellular internalization in cancer cells and macrophages occurred via transporters or endocytosis (mechanism of action). Membrane transporters including organic anion transporting polypeptides (OATPs), organic cation transporters (OCTs/OCTNs), and ATP-binding cassette (ABC) transporters regulate the transcellular movement of small organic molecules and are implicated in playing a critical role in tumor targetability of organic fluorophores. Endocytosis also represents a major pathway for cellular uptake of diagnostic and therapeutic nanoparticles. Neither bromosulphophthalein, cyclosporin A (both OATP inhibitors), D22 (OCT inhibitor), nor MK-571 (ABC transporter inhibitor) significantly inhibited SH1 uptake (FIGS. 10A-B), suggesting that these membrane transporters were not involved in this process. Dynamin-dependent endocytosis inhibitor dingo 4a significantly inhibited the internalization of TAIC targeted SH1 and decreased accumulation in all the cell types tested. In contrast, Pitstop 2, which is a clathrin-dependent endocytosis inhibitor, did not result in any appreciable change in cellular uptake. These results suggest that fast endophilin-mediated endocytosis (FEME), which is a clathrin-independent/dynamin-dependent subgroup pathway of endocytosis, plays a predominant role in cellular uptake of SH1, and that SH1 has great potential for broad-spectrum immune cell targeting as well as tumor targetability via FEME. In addition to the inhibition test, a cell viability assay was conducted which showed that up to 10 μM of SH1 did not affect cell proliferation (FIG. 10C). A flow cytometry experiment was conducted to evaluate the target cells in TAICs in bone marrow and tumors. It was found that the majority of the population which was targeted by SH1 were immune-related cells of the bone marrow such as macrophages, monocytes, lymphocytes, neutrophils, and dendritic cells (FIGS. 3A and 3B, gating strategy is shown in FIG. 11). Among them, macrophages, Ly6C^{low} monocytes, and dendritic cells were identified as SH1-targeted TAICs in the tumor region. In addition, significant inflammatory infiltrations of targeted immune cells, as well as TRICs, were observed by immunohistochemical staining (F4/80 antigen as a mouse macrophage marker) and histological fluorescence analyses, which resulted in a significant increase in the fluorescent signals in tumor tissues (FIG. 3C).

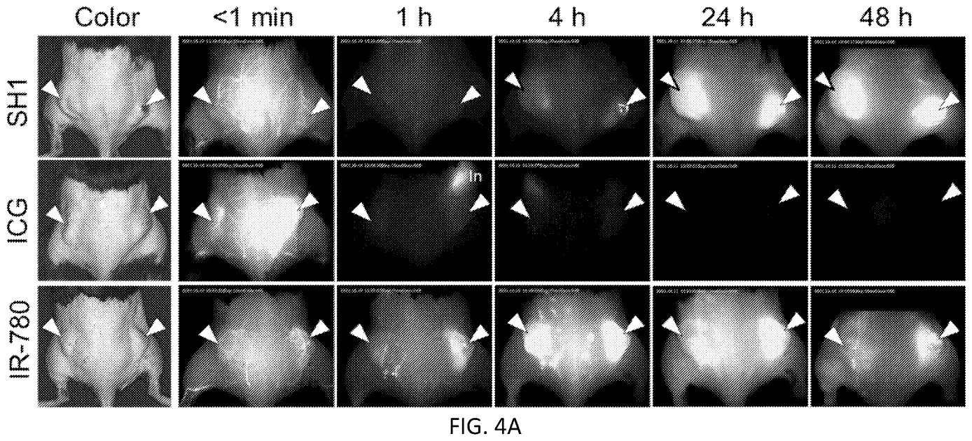

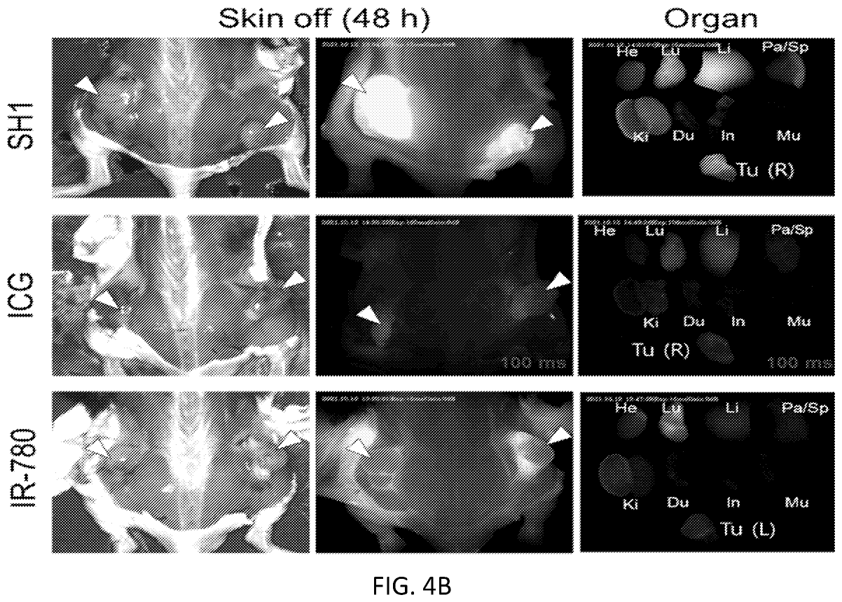

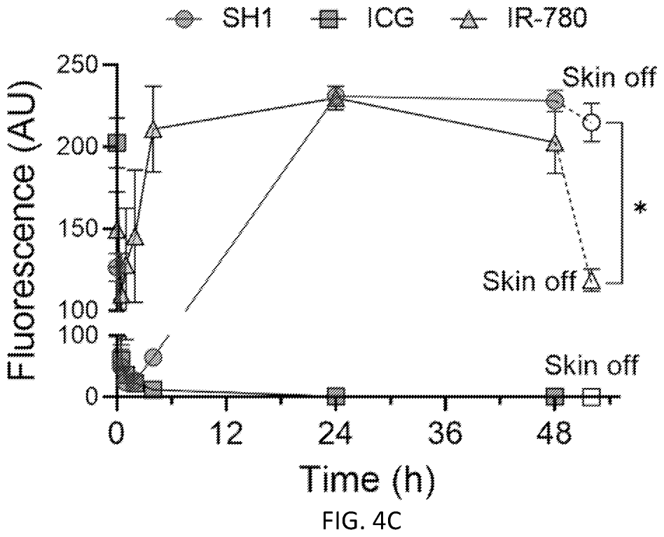

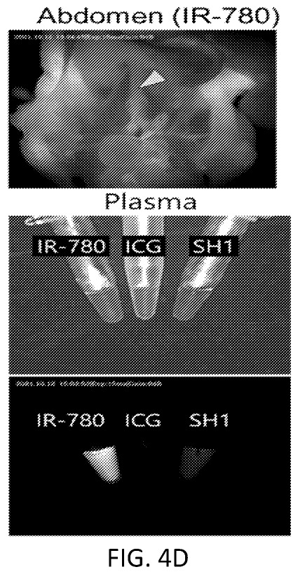

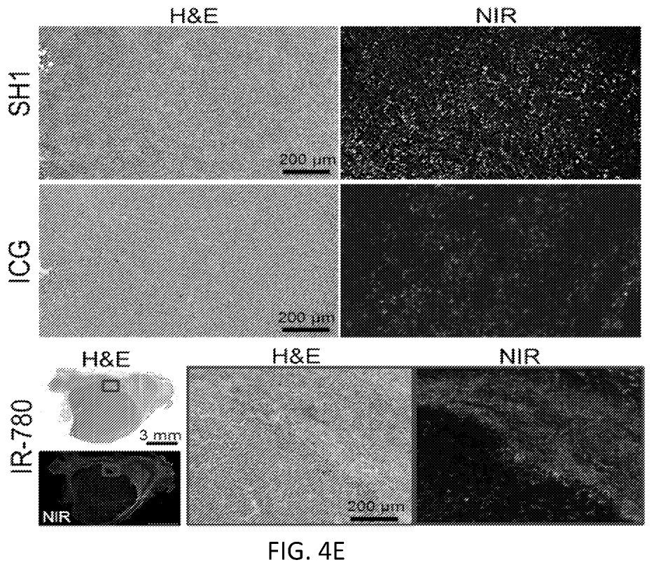

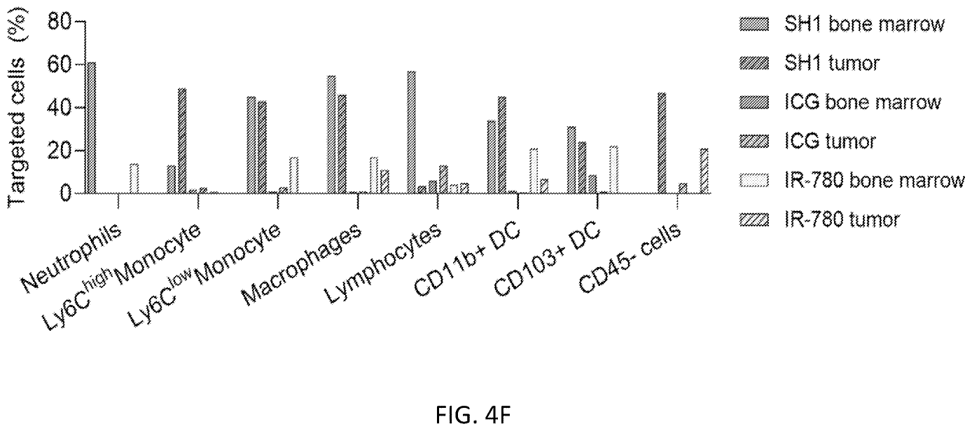

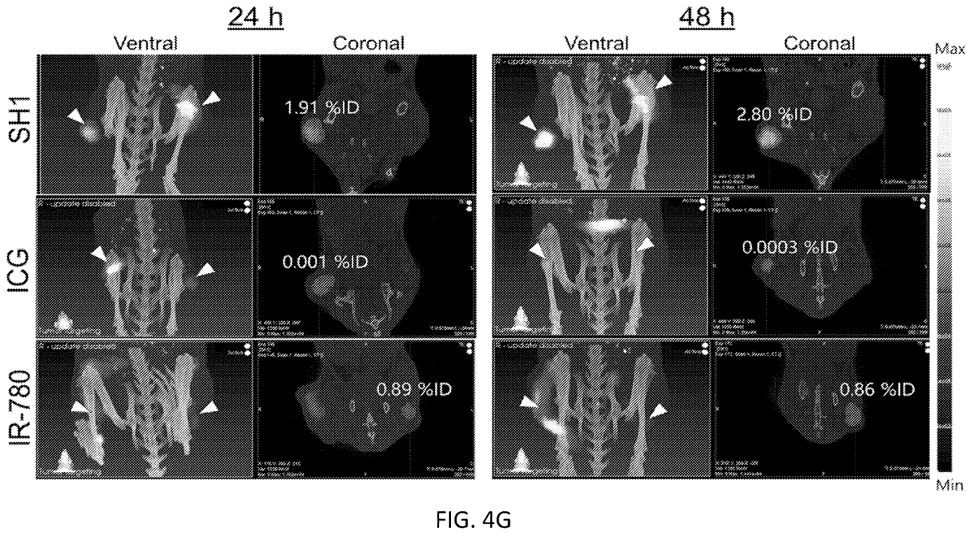

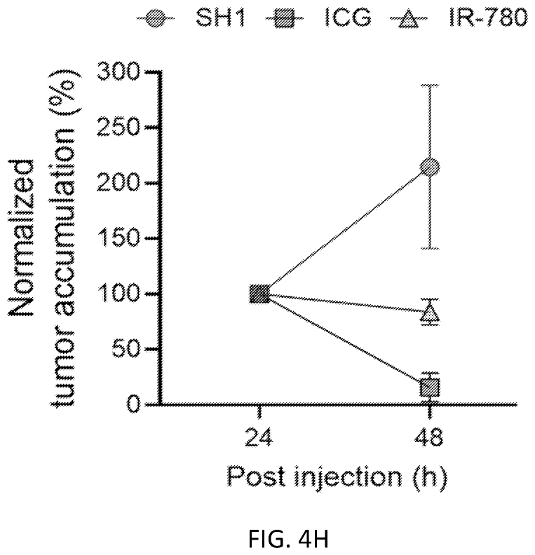

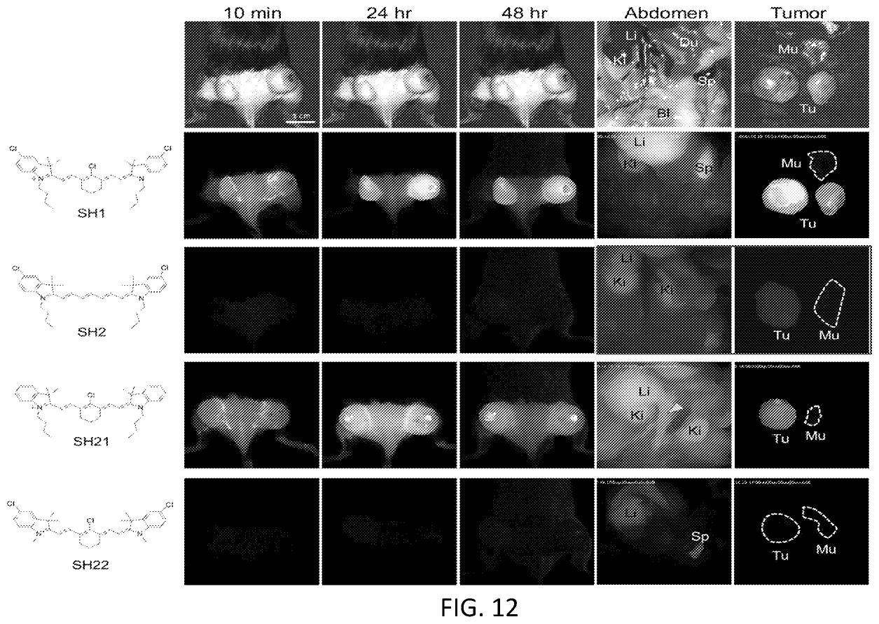

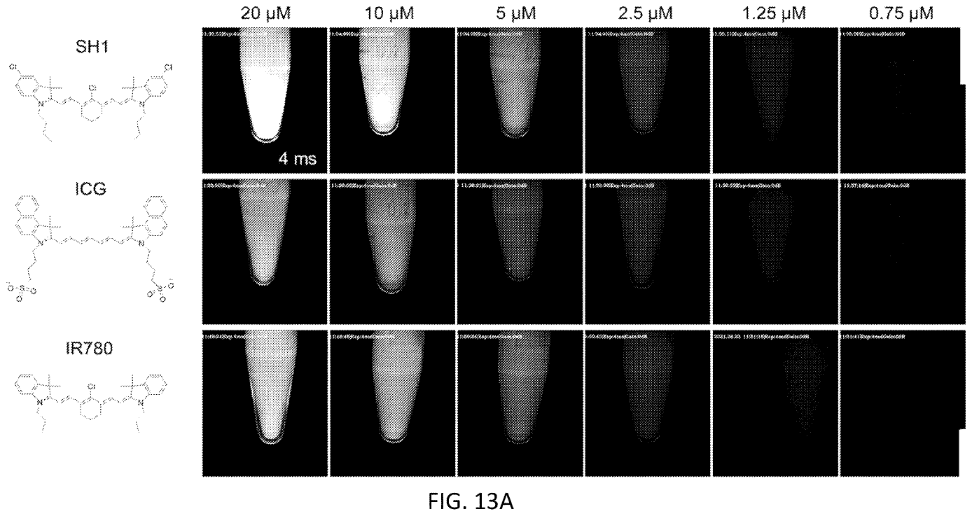

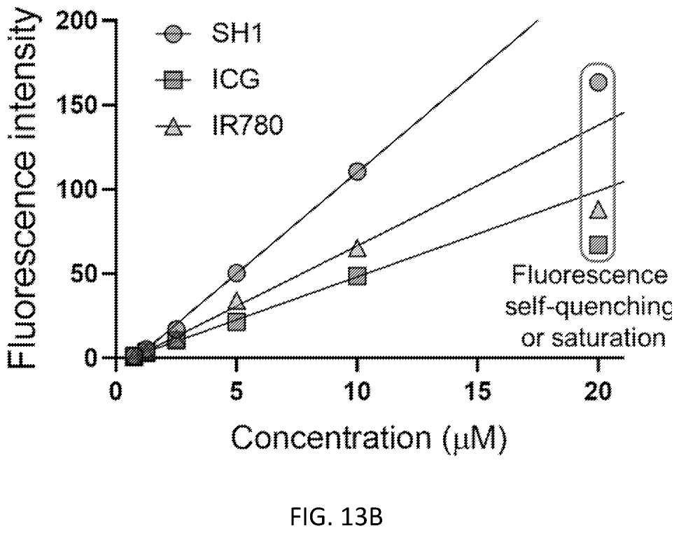

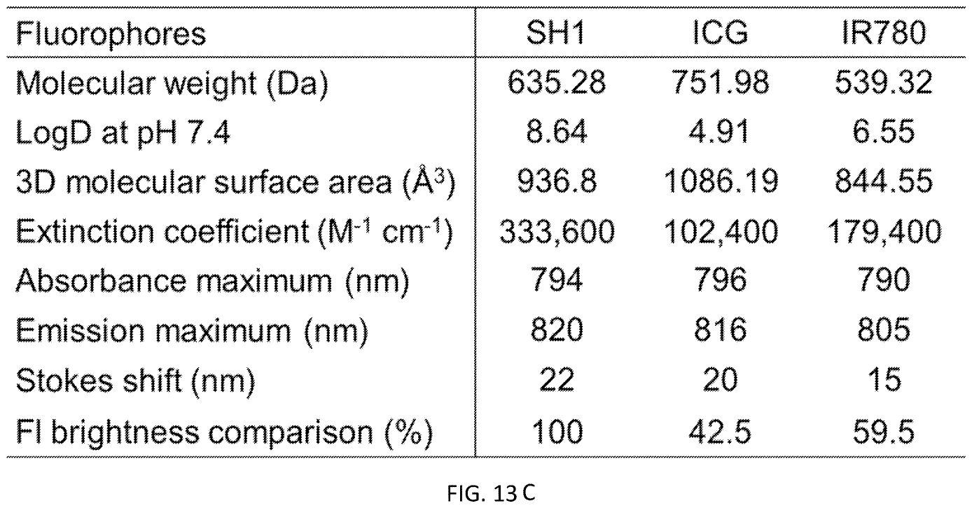

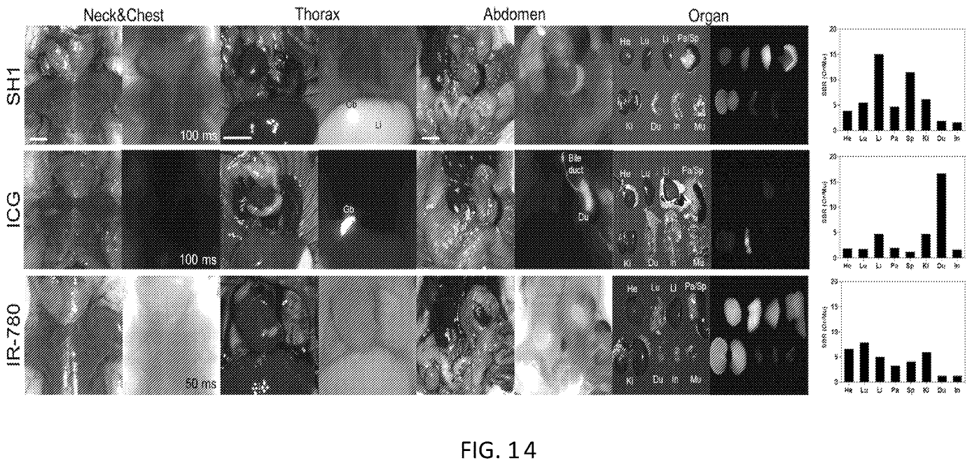

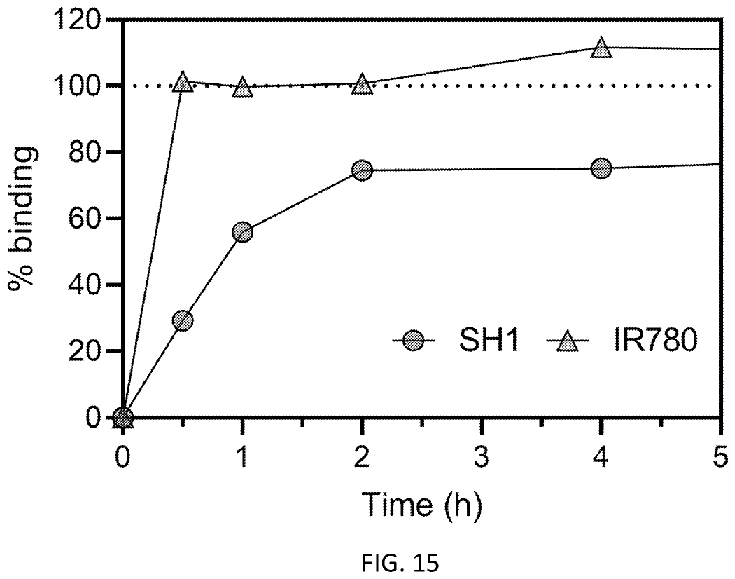

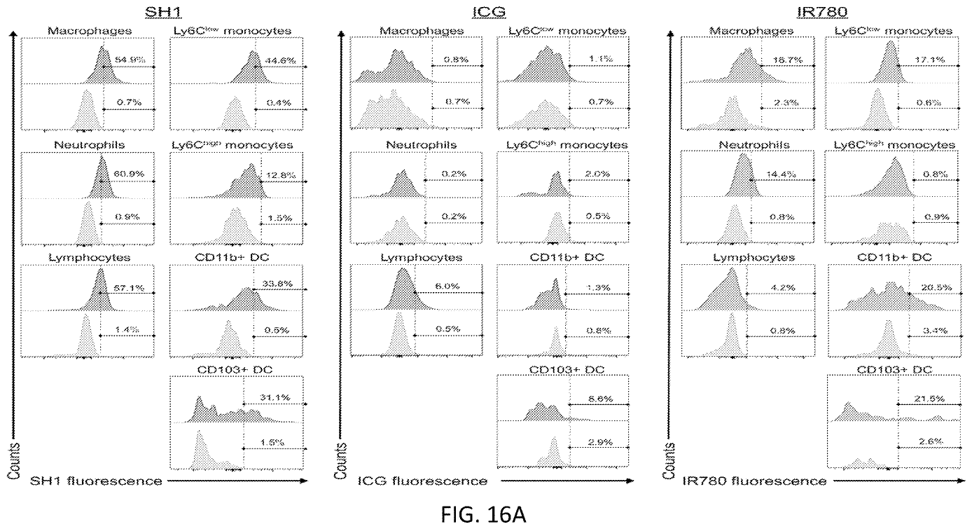

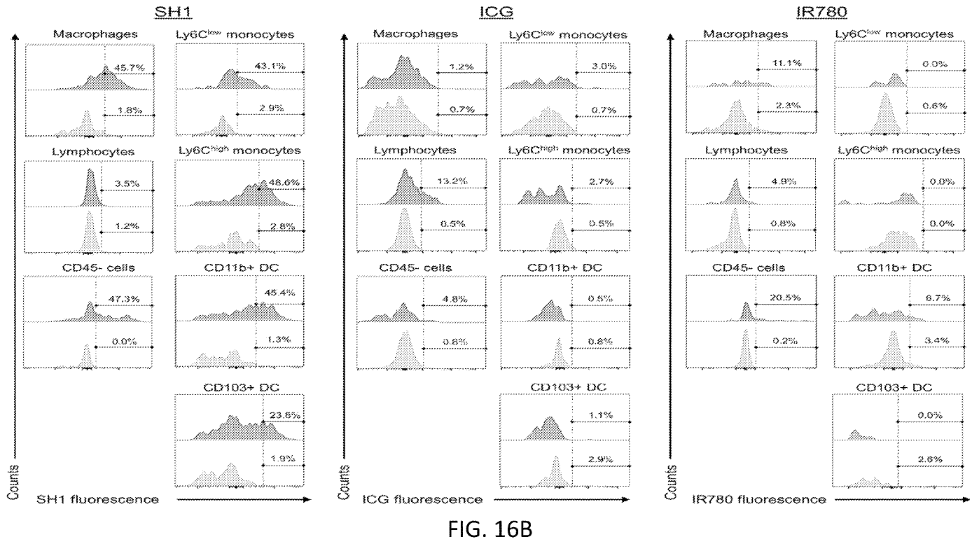

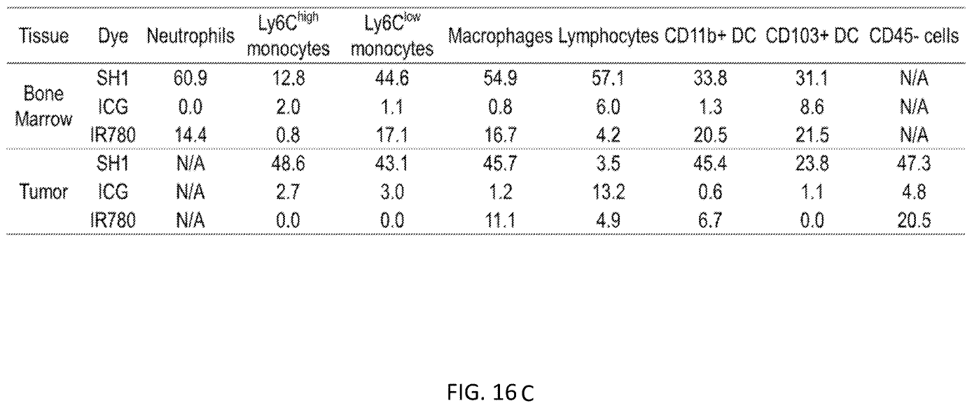

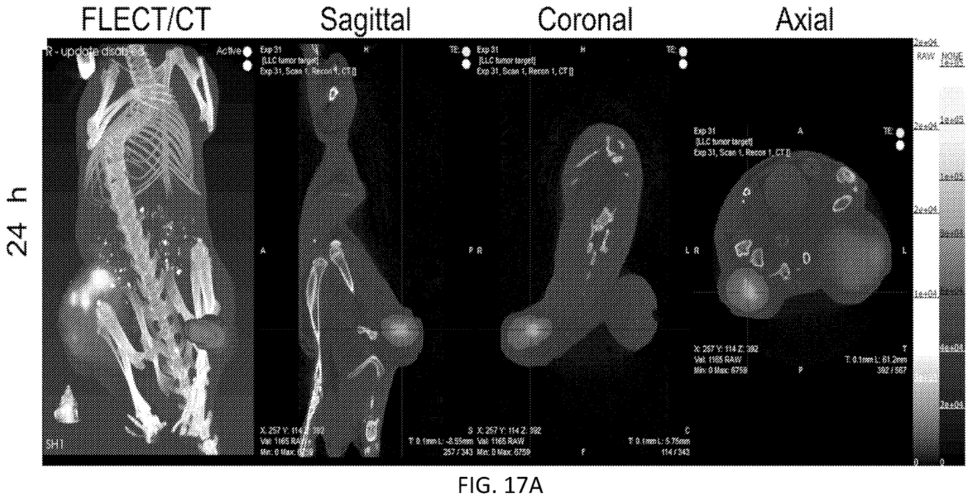

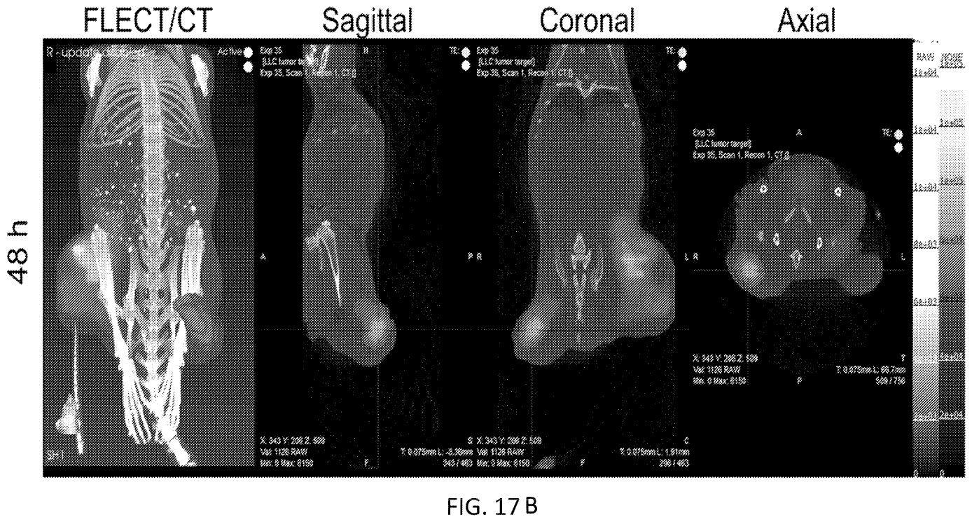

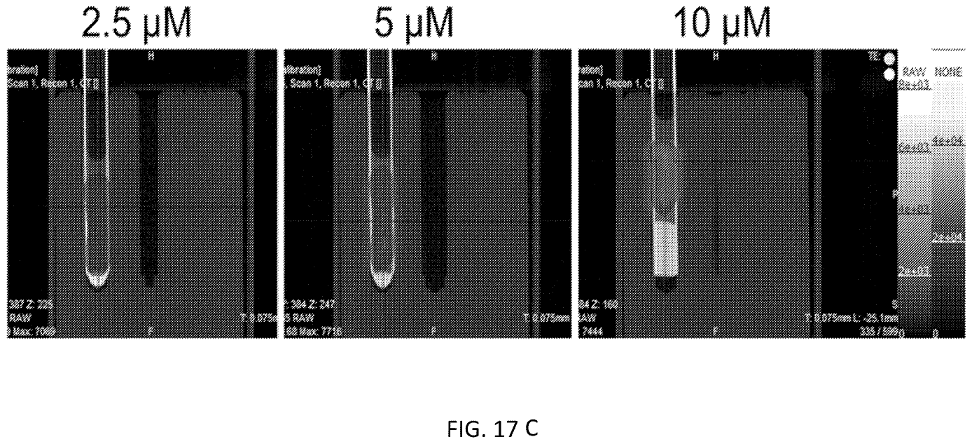

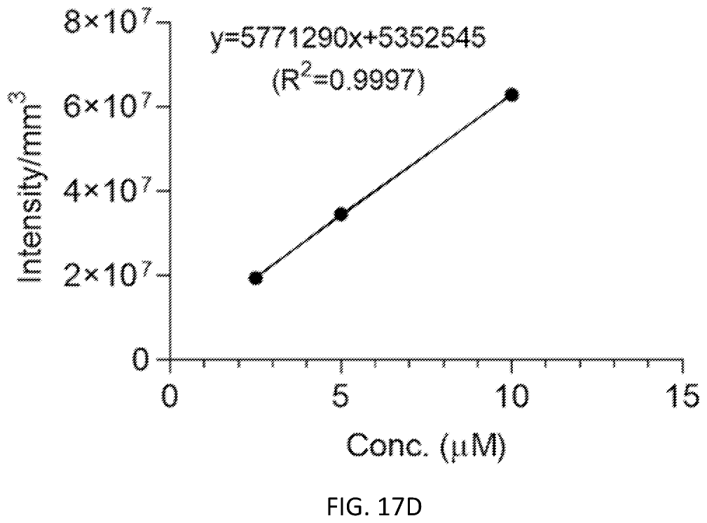

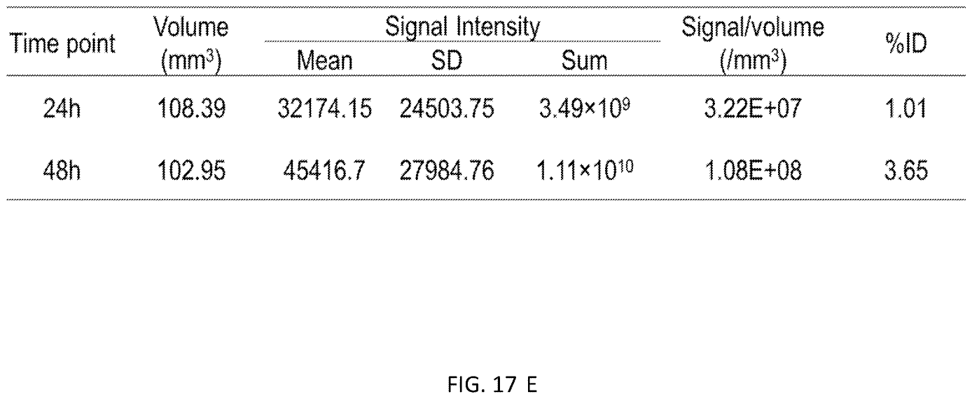

Next, the structural effect on in vivo tumor targetability were investigated to verify whether the mode of transport to tumors relied on the immune cell-mediated tumor targeting of SH1 rather than the EPR effect by comparison with the SH1 analogs and previously reported tumor targeting fluorophores, ICG and IR-780, as controls. First, all SH series fluorophores were intravenously injected into LLC tumor-bearing mice to confirm the structural effects. Interestingly, the dyes which had only chlorine atoms or butyl side chains did not show strong fluorescence signals in tumor sites, as shown in FIG. 12. This result indicates that both chlorine atoms at the meso/side positions and butyl chains together are important for tumor targetability based on the TAIC targeting strategy. Second, SH1, ICG, and IR-780 were compared in terms of the NIR-II fluorescence brightness, tumor targeting mechanism, and targeting capability by using NIR-II imaging, 3D FLECT/CT quantification imaging, flow cytometry, histological, and serum protein binding analyses. As the result of brightness in the NIR-II region, SH1 is 2.2- and 1.7-fold brighter than ICG and IR-780, respectively (FIGS. 13A-C). SH1, ICG, and IR-780 were injected into LLC tumor-bearing mice, and SH1 showed superb signal intensity in tumor area at 48 h post-injection while ICG did not show good tumor targeting owing to its fast hepatobiliary clearance (FIGS. 4A-C, FIG. 14). IR-780 showed a different tumor accumulation pattern from that of SH1; accumulating in tumors at early time points around 4 h post-injection. In addition, even though similar signal intensity in tumors was observed in IR-780 and SH1 injected mice, the signal intensity in IR-780 injected mice significantly decreased after skin removal (FIG. 4C). This different tumor accumulation indicated that the tumor targeting mechanism of IR-780 (especially the mode of transport) was different from that of SH1. The strong signals in the blood plasma of IR-780 injected mice observed at 48 h post-injection were likely due to fast/permanent serum protein binding of IR-780 (FIG. 4D, FIG. 14) which was confirmed by serum protein binding test (FIG. 15). Unlike with IR-780 (100% serum protein binding, mostly albumin, within 30 min; the time to bind half of the maximum was reported to ˜2 min), SH1 showed relatively slow serum protein binding kinetics (˜35% within 30 min) which caused significant differences in the initial biodistribution, target organs, and tumor targetability. In FIG. 4E, histological and flow cytometry analyses further confirmed the different tumor targeting patterns/abilities among SH1, ICG, and IR-780 (FIG. 4E-F, FIG. 16). ICG signals were barely present in the tumor, and IR780 signals were only present in the peritumoral region, but SH1 gave strong fluorescence signals in the targeted tumor. In addition, ICG barely targeted immune cells as positively stained cells across distinct immune cell populations were <8% in the bone marrow and <14% in the tumor. Similarly, IR-780 poorly targeted immune cells as those were <22% positive in bone marrow and <7% in the tumor. In contrast, SH1 targeted tumor-associated immune cells (Ly6C^{high} monocyte; 48.6%, Ly6C^{low} monocyte; 43.1%, Macrophage; 45.7%, CD11b+ DCs; 45.4%, CD103+ DCs; 23.8%). Additionally, the % injection doses (% ID) of each injected dye in cancer areas at 24 and 48 h were estimated based on a three-dimensional (3D) tomographic fluorescence imaging technique using the InSyTe™ FLECT/CT system (TriFoil Imaging, Chatsworth, CA) (FIG. 4G-H, FIG. 17A-B). The % ID of SH1 increased more than 2-fold from 24 h to 48 h post-injection, which further suggested that SH1 targeted TAIC migration increases TBR over time.

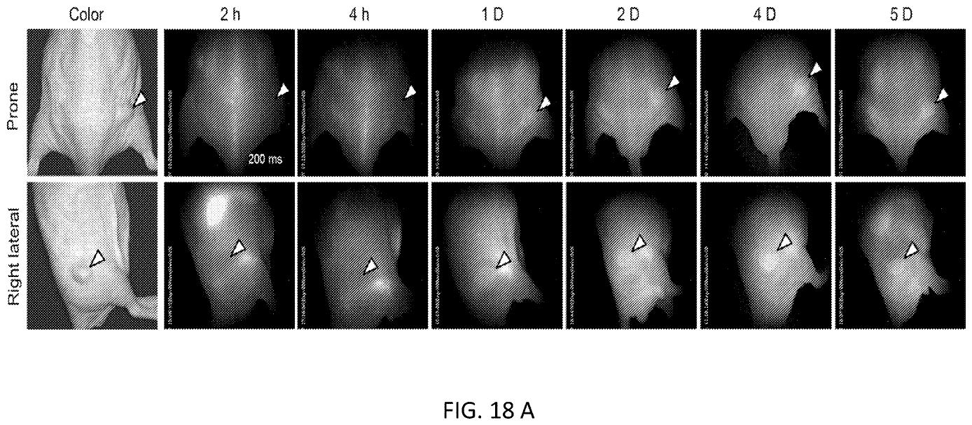

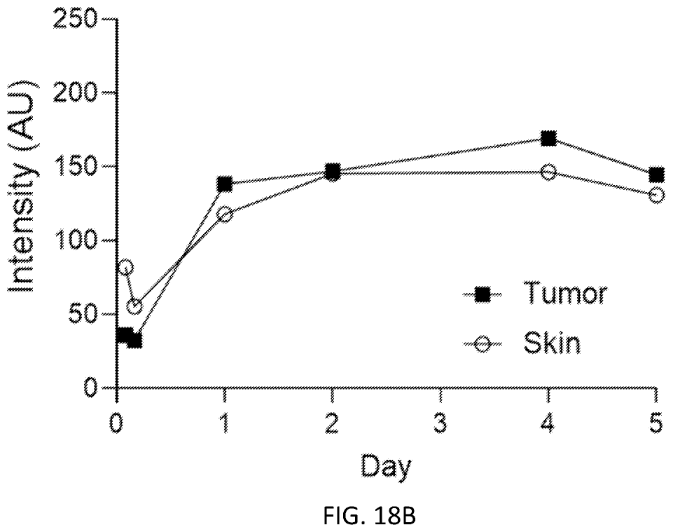

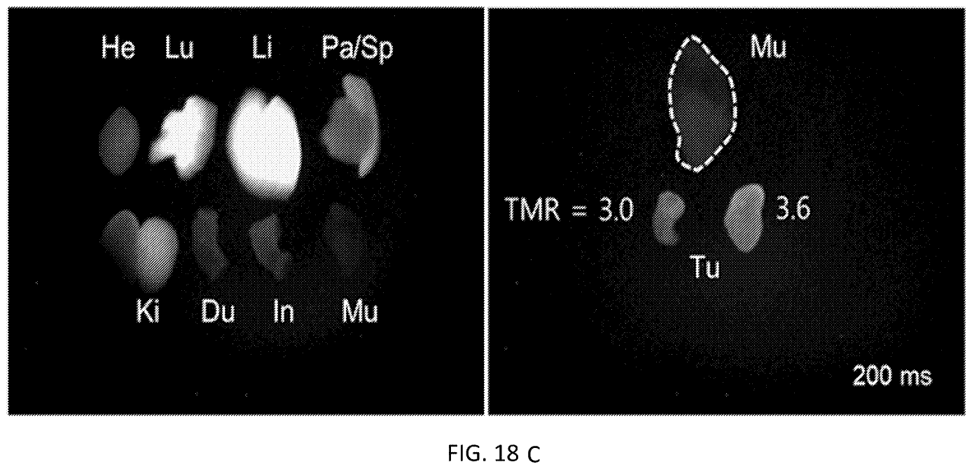

Lastly, tumor targeting of SH1 was performed on Pan02 cell inoculated immune-deficient athymic nude mice (NCr nude homozygous CrTac: NCr-Foxn1^{nu}) (FIG. 18A-C). The SH1-injected mice showed high uptake in the immune-related cells of bone marrow initially, of which signals decreased gradually after 1-day post-injection. However, the skin background signal increased instead of the tumor signal which might imply immune cells were not recruited to the tumor likely due to the immune deficiency. As a result of the high background, the tumor-to-muscle signal ratio was calculated to be ˜3.0. This result of tumor targeting on immune-deficient mice strongly supported that SH1 tumor targetability was associated with SH1 targeted immune cell migrations from the bone marrow to the tumor area. Overall, the results of various analyses support the TAIC-mediated tumor targeting mechanism for SH1 as the mode of transport to tumor which occurs via 1) direct targeting to tumor cells as well as TAICs such as macrophages, monocytes, lymphocytes, neutrophils, and dendritic cells, and 2) migration of targeted TAICs through blood vessels followed by infiltration to a cancerous region.

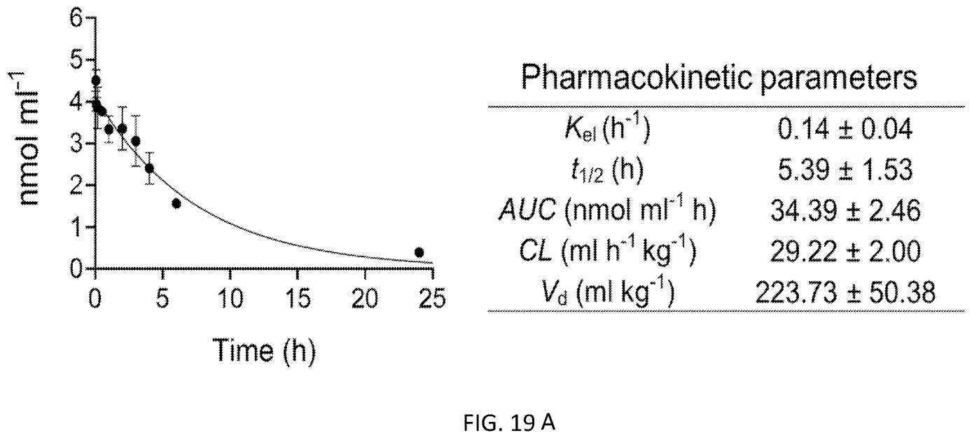

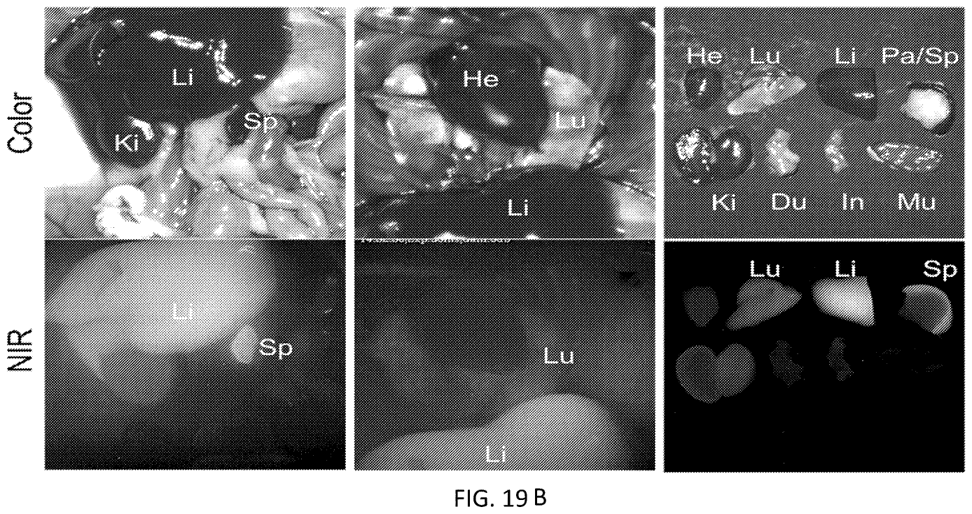

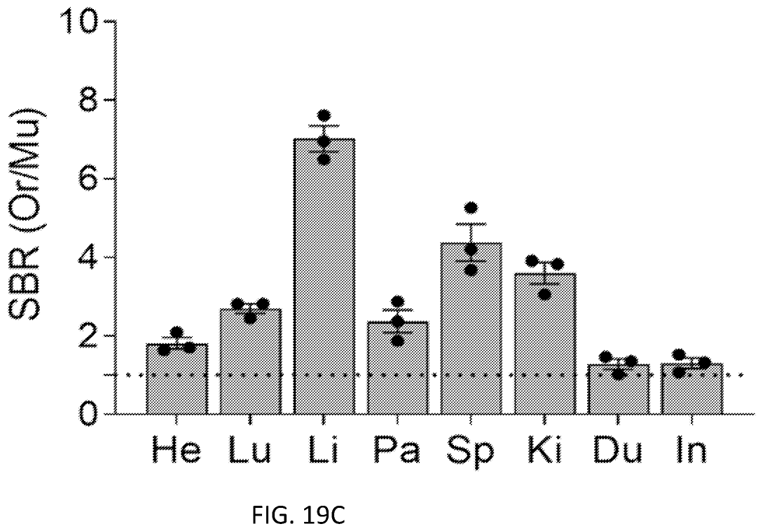

To examine the pharmacokinetics and biodistribution characteristics of the TAIC targeted fluorophore SH1, CD-1 mice were intravenously administered SH1 (1 μmol/kg). Blood concentration in serum and biodistribution was quantified by measuring the fluorescence signal (FIG. 19A-C). The results demonstrated that SH1 followed a one-compartment model and distributed completely within 24 h (t½=5.39±1.53 h), with major distribution in the liver and spleen which are immune cell abundant organs.

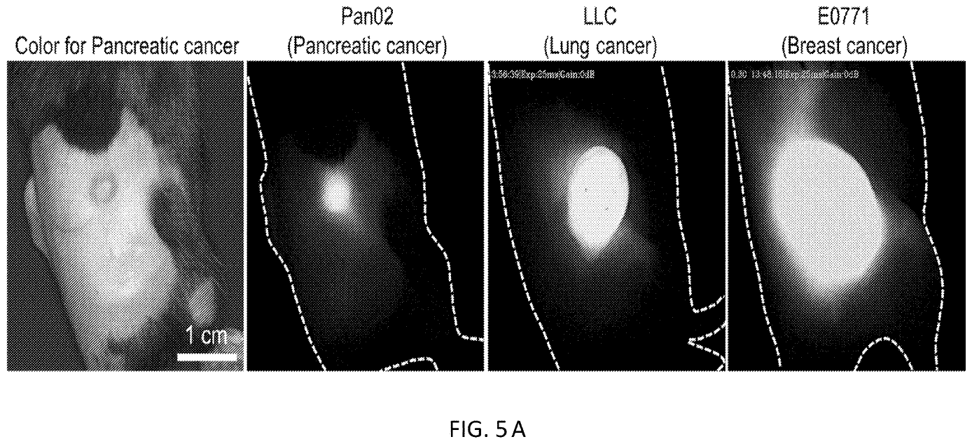

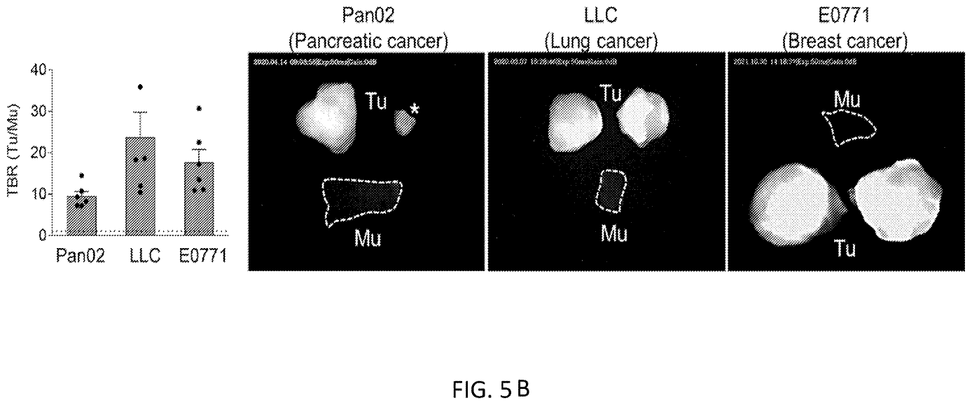

SH1 could potentially target varying types of tumors due to its universal targeting mechanism. To evaluate its broad-spectrum tumor targeting, three syngeneic tumor models with different tumor types and sizes including pancreatic, lung, and triple negative breast cancers were established (FIG. 5A-B). The syngeneic mouse models which have a functional immune system presented the tumor development, the microenvironment, and the immune response. Despite the absence of specific targeting moieties, SH1 had excellent targeting performance in pancreatic ductal adenocarcinoma (PDAC) less than 5 mm in size with high TBR (˜9.5) compared to the healthy tissue region. In larger-sized tumors, TBRs were 23.7 and 17.6 in lung and breast cancers, respectively. This suggested that SH1 showed broad spectrum tumor-targeting capability, which was independent of cancer type and size.

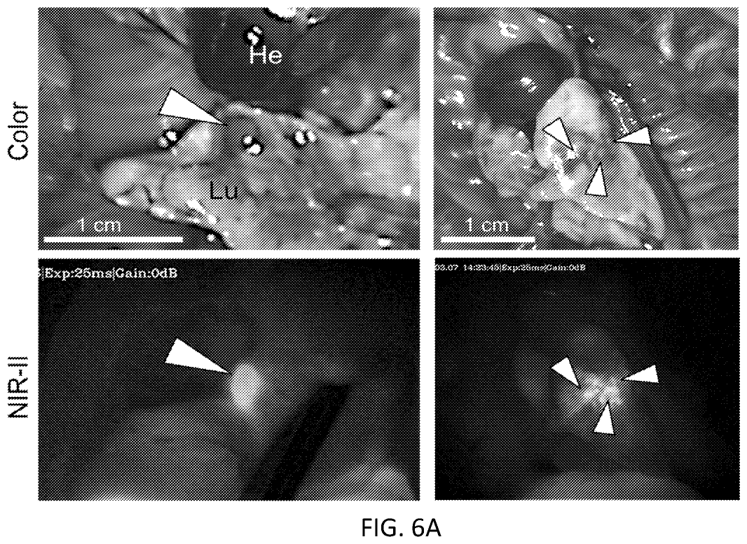

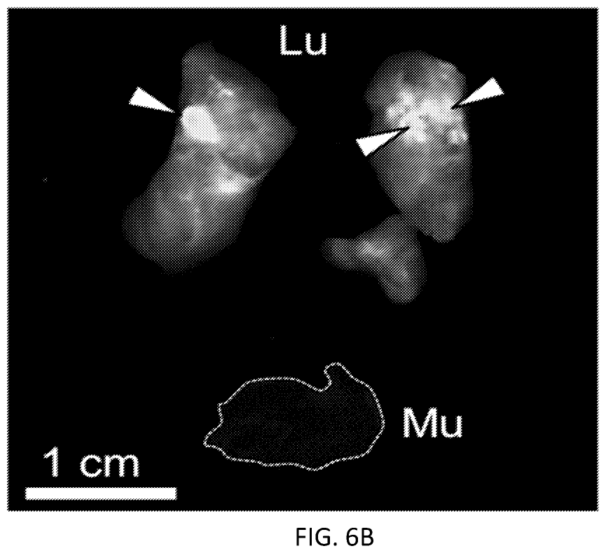

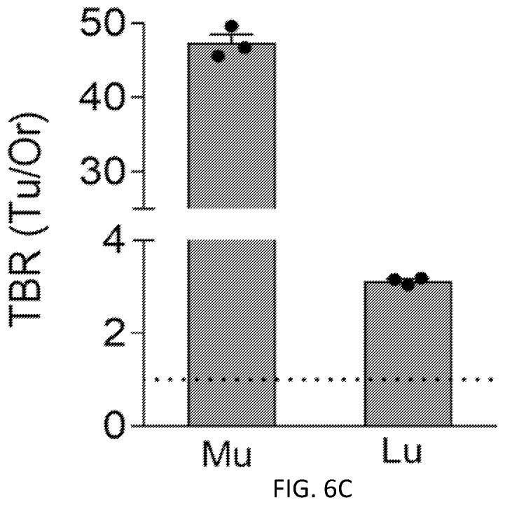

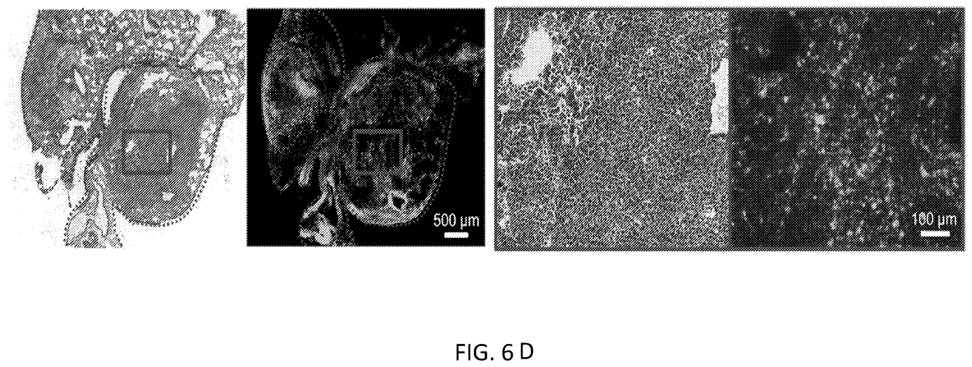

The tumor targetability of SH1 in an orthotopic lung tumor model was evaluated. This model could provide a reliable representation of the tumor environment as cells were placed directly into their organ of origin. Fluorescence signals from the small tumors were clearly shown in a real-time image (FIG. 6A). The high TBRs 47.3 and 3.12 were calculated against normal tissue and the lung, respectively, suggesting SH1 had significant potential as a contrast agent in intraoperative thoracic surgery. It was also confirmed the margin in the dissected tumor can be distinguished by the strong fluorescence signals of SH1, and it is well-correlated with the H&E staining result (FIG. 6D)

In conclusion, in this example, TAIC-targeted fluorophore SH1 as a SITT agent for intraoperative NIR-II fluorescence imaging were designed and synthesized. The TAIC-mediated tumor targeting mechanism was confirmed by flow cytometry and histological studies, in vivo NIR-II fluorescence imaging, and 3D tomographic imaging. The NIR-II capability of SH1 along with the InGaAs camera built-in NIR-II imaging system greatly facilitated image quality permitting the observation of signals in deep tissues and significantly improved the sensitivity in intraoperative cancer surgery. The SH1 fluorophore could reach a high TBR (9 to 47 in various cancer types) in tumor sites in comparison with healthy tissue. Furthermore, SH1 could also be used to detect small lesions such as metastatic tumors. Thus, the compounds of this disclosure are cancer-targeting agents useful in intraoperative optical imaging.

### Example 6—Physicochemical Properties of Exemplified Compounds

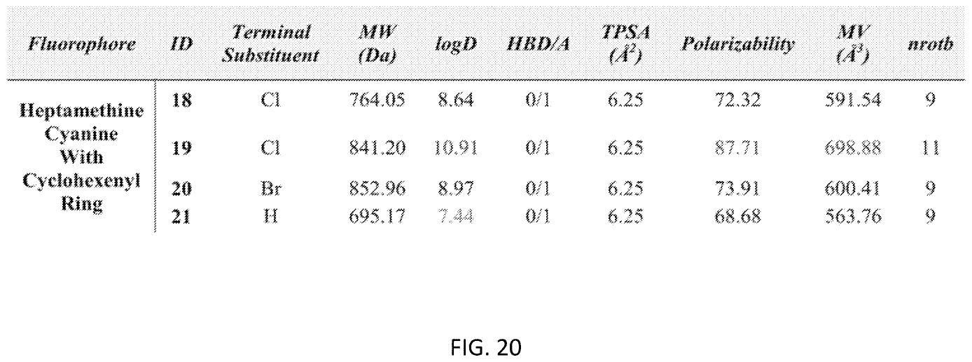

In silico calculations of physicochemical properties, which included well-defined rotatable bonds, hydrogen bond donor-acceptor groups, hydrophobicity, and polar surface areas, can correlate the relationship between an optimized structure and its biological activities, such as permeability, retention, and clearance. Modifying the structures of any small molecules can alter these properties. For example, for an increased lipophilicity of a certain fluorophore at a certain pH, log D (distribution coefficient) predicts that molecules will partition faster into lipid cell membranes. The topological polar surface area (TPSA) predicts intestinal absorption. Higher TPSA means low permeability because of more retention in the vascular system, such as a larger molecular volume greater than 40 kDa can increase the residence time in the peripheral compartments. However, the low MW can increase the permeability. Therefore, to predict the localization of the fluorophores in a specific tissue, permeability, cellular uptake, retention, and clearance the physicochemical properties were examined by ChemAxon (JChem plugin). The results obtained from in silico calculations are presented in FIG. 20. The molecular volumes of the synthesized fluorophores lied between 564 and 699 Å³, while their TPSA remained the same at 6.25 Å². The log D values at pH 7.4 for the fluorophores 18-21 (as labeled in Example 4 and FIG. 7D) are fallen between 7.44 and 10.91, and the polarizability value changes from 69 to 88. Among the log D values presented in FIG. 20, fluorophore 21 showed the lowest hydrophobicity value, whereas fluorophore 19 showed the highest.

The data calculated (at pH 7.4) include log D, polarizability, number of rotatable bonds (nrotb), molecular volume (MV, Å³), topological polar surface area (TPSA, Å²), and molecular weight (MW, Da).

In some embodiments, a large alkyl chain increases hydrophobicity. In addition to large alkyl chains (phenyl propyl), fluorophore 19 also has three Cl atoms in the structure and a cyclohexenyl ring in the middle, and had the highest log D at pH 7.4. Although TPSA remains the same for all of the fluorophores, polarizability and molecular volume change with the structures. We observed a linear relationship between molecular volumes and polarizabilities. Polarizability increased with the increment of molecular volume. Fluorophore 19 is the most polarizable among the fluorophores with its largest molecular volume (698.88 Å³).

### Example 7—Optical Characterization of Exemplified Compounds

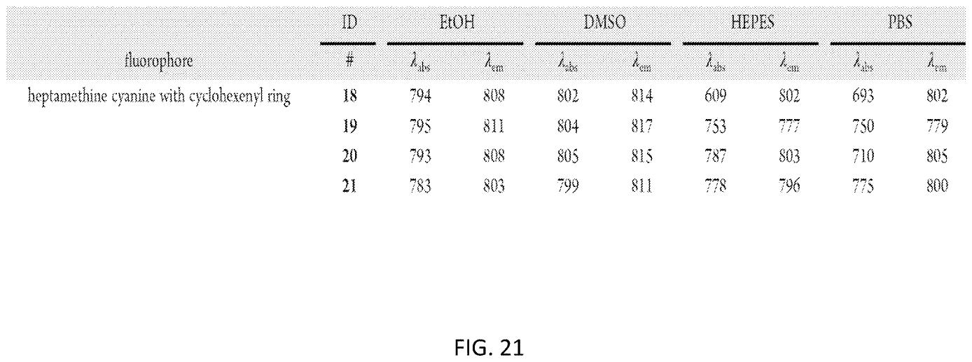

In vivo success of a contrast agent can depend on the absorption and emission profiles, molar extinction coefficient, quantum yield, molecular brightness, physicochemical properties, and photochemical stability. Different types of polymethine bridges can have an impact on the structural geometry of the fluorophores and the absorption and emission profiles. Optical properties were measured in polar protic EtOH, polar aprotic DMSO, and two buffer solutions, phosphate-buffered saline (PBS, PH ˜7.4) and 4-(2-hydroxyethyl)-1-piperazine-ethanesulfonic acid (HEPES, PH ˜7.4). Organic solvents such as ethanol (EtOH) and dimethyl sulfoxide (DMSO) are often used in spectroscopic experiments to observe the absorption and emission maximum, whereas buffer solvents PBS and HEPES were used to mimic the biological environment, as these synthesized fluorophores are intended to be used in bioimaging. The absorbance profiles of these fluorophores were recorded at various concentrations (0.8, 1.6, 2.4, 3.2, 4.0 μM) and the absorbance values obtained were plotted against each of the concentrations. The measured media showed a linear correlation between absorbance and concentration, in accordance with the Beer-Lambert law. Measured absorption and emission profiles are summarized in FIG. 21. Fluorophores 18-21 refer to the labels in Example 4 and FIG. 7D.

Heptamethine cyanine with cyclohexenyl ring 19 exhibited the highest emission wavelength at 817 nm in DMSO. The absorbance maxima red-shifted by 5-20 nm in DMSO but blue-shifted (20-130 nm) in buffer solutions. The emission spectra can be highly influenced by the nature of the media, and large spectral shifts are observed in buffer solutions. Absorbance maxima (λₘₐₓ) of heptamethine cyanine fluorophores 18-21 with cyclohexenyl rings were observed in the range of 661-805 nm. Different substituted groups, such as chlorine and bromine, had a negligible effect on the dye absorption and emission spectra. Upon excitation at the absorbance maximum, 661 nm, the emission wavelength of fluorophore 18 in HEPES buffers was seen at 802 nm, which gave the largest Stokes shift, 140 nm, observed among these fluorophores.

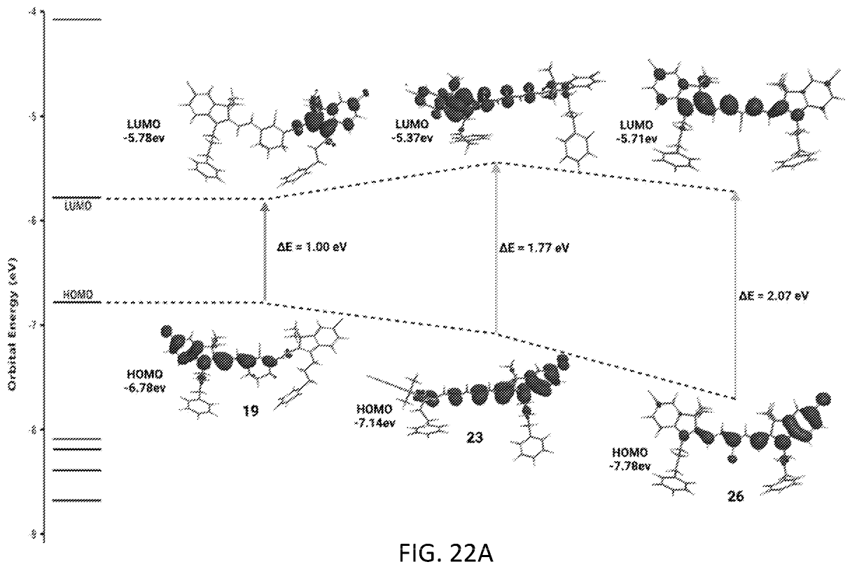

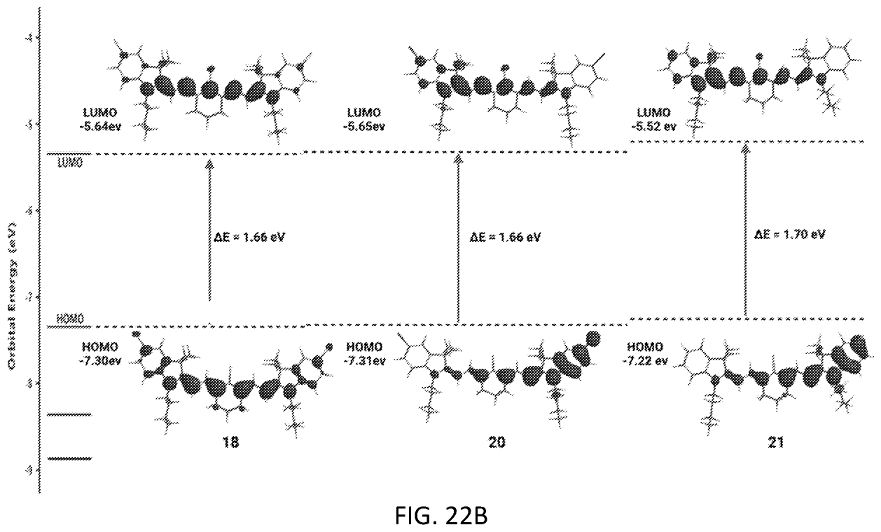

To understand the contribution of the geometry and the pi system on the optical properties, quantum chemical calculations were performed on the selected fluorophores by frontier molecular orbitals (FMOs) in optimized structures based on the density functional theory (DFT) method with a 6-311G basis set. In comparison to pentamethine, the presence of one extra double bond conjugation in heptamethine fluorophores introduced more vibrational energy levels and decreased the energy difference between the ground states and the excited states. Moreover, the presence of a cyclohexenyl ring in the polymethine bridge enhances rigidity, made the overall structure flat, and decreased the energy band gap between FMOs. FIG. 22A shows the energy difference of optimized molecular structures between FMOs of fluorophore 19 and two comparative fluorophores 23 (open chain heptamethine cyanine) and 26 (pentamethine cyanine). The energy differences between the highest occupied molecular orbitals (HOMOs) and lowest unoccupied molecular orbitals (LUMOs) increased from 1.00 to 2.07 eV. These fluorophores have the same terminal indolium heterocycles but different polymethine chains such as fluorophore 19 with a cyclohexenyl ring in the polymethine bridge, fluorophore 23 with an open polymethine chain, and fluorophore 26 with one double bond shorter conjugation length polymethine chain. As reported, energy is inversely proportional to its wavelength. The wavelength for absorption and emission maxima decreased as following fluorophores 19λ_{abs}/λₑₘ>23λ_{abs}/λₑₘ>26λ_{abs}/λₑₘ with the increase of the energy difference from left to right as shown in FIG. 22A.

DFT calculations were performed on fluorophores 18, 20, and 21 presented in FIG. 22B to understand the substituent effect on the absorption and emission wavelengths. We observed that the substituents, such as the halogen atoms (Cl/Br), do not affect the energy gap, whereas the presence of a hydrogen atom changes the HOMO-LUMO gap slightly. Therefore, fluorophore 18, having a substituent Cl atom in the heterocyclic rings, and fluorophore 20, containing a Br atom in the heterocyclic rings, showed almost similar absorption and emission wavelengths. But fluorophore 21, without any halogen substituent showed slightly less absorption and emission wavelengths. This outcome can be explained by the resonance and induction effect of the halogen atom. Halogen atoms in the heterocyclic rings possibly increase the electron density by pushing electrons to the conjugation system. On the other hand, it stabilized the conjugation of the polymethine bridge by withdrawing electrons through an inductive effect. This phenomenon pushes the overall HOMO and LUMO gap narrower. Without wishing to be bound by theory, halogen atoms substituents in the heterocyclic rings and in the polymethine bridge can provide a little more stable structures with higher absorption and emission maxima than their substituent hydrogen atoms.

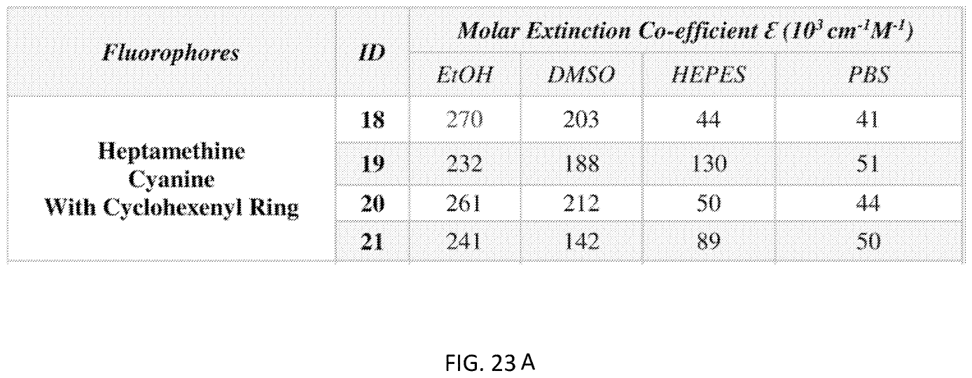

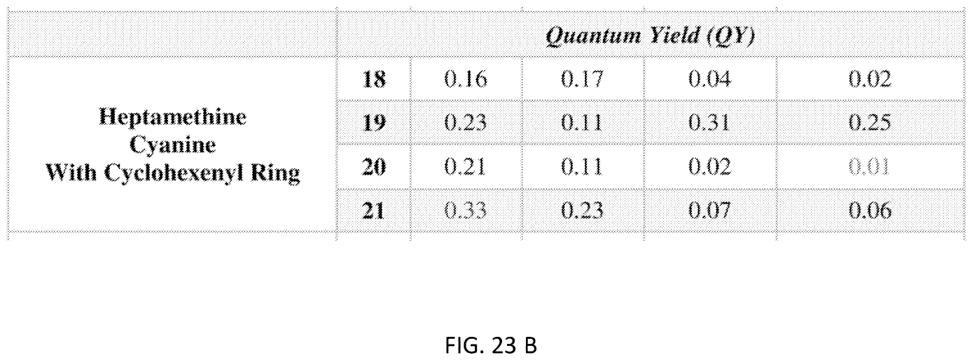

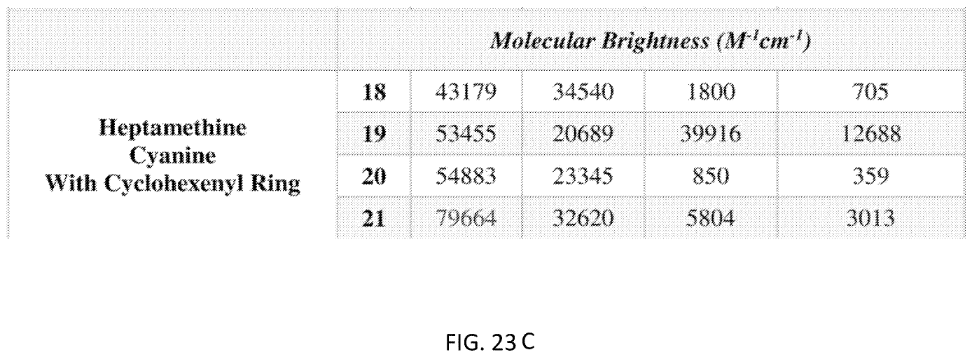

The molar extinction coefficient is an intrinsic property of any fluorophore and can be important for high image resolution. Generally, fluorophores with high molar absorptivity and quantum yield showed high molecular brightness, which means that less dosing is required for in vivo applications. These properties vary with the structure change and are difficult to predict beforehand. The optical properties of the synthesized fluorophores were measured in various media. The data is summarized in FIGS. 23A-C. All measurements were performed in 2 different organic solvents (EtOH and DMSO) and 2 different buffer (HEPES and PBS), and ICG was used as a reference (Φ_{ICG}=0.043).

Synthesized fluorophores showed higher molar extinction coefficients and molecular brightness in organic solvents but lower extinction coefficients and brightness in buffer solutions. Due to their hydrophobic nature, all of the synthesized fluorophores tend to aggregate when in aqueous environments. This aggregation causes a reduction in their molar extinction coefficient and molecular brightness, ultimately affecting their ability to absorb and emit light. Furthermore, the aggregated state of the dye molecules can result in fluorescence quenching, which further diminishes their brightness. The molar extinction coefficients of fluorophore 18 were observed in the decreasing order 270,000, 203,000, 44,000, and 41,000 cm⁻¹ M⁻¹ in EtOH, DMSO, HEPES, and PBS, respectively (see FIG. 23A). Fluorophore 18 showed significantly lower quantum yield and molecular brightness in buffer solutions (see FIGS. 23B and 23C). The highest molar extinction coefficient (270,000 cm⁻¹ M⁻¹) was observed for fluorophore 18, while the highest molecular brightness (79,664 cm⁻¹ M⁻¹) was observed for fluorophore 21, and the highest quantum yield (0.33) was observed for fluorophore 21 in EtOH. The lowest quantum yield (0.01) was observed for fluorophores 20 and 21 in PBS. Furthermore, it was observed that heptamethine cyanine dyes had higher extinction coefficients in organic solvents than pentamethine cyanine dyes.

### Example 8—Photostability of Exemplified Compounds

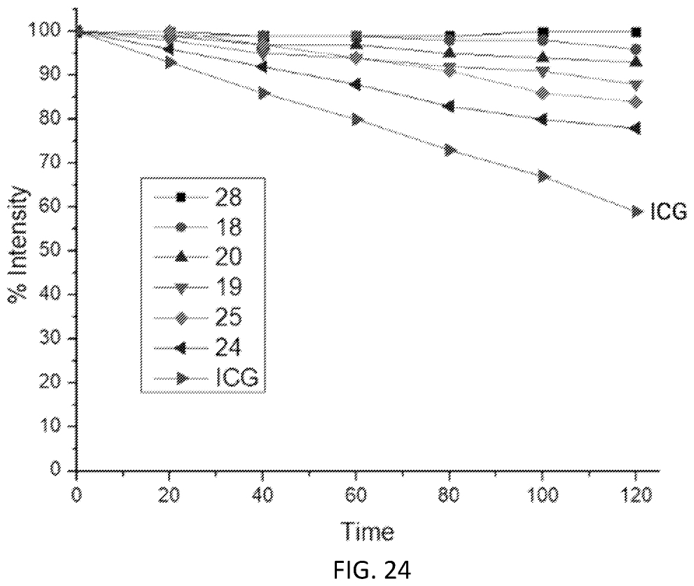

In consideration of theranostic (therapeutics and diagnostic) applications, NIR cyanine fluorophores are synthesized for fluorescence imaging, photodynamic therapy (PDT), photothermal therapy (PTT), optoacoustic imaging, and cancer immunotherapy. One of the approaches before using these fluorophores in those applications is to study their biodistribution and clearance pattern in vivo. Generally, after intravenous injection of these contrast agents, 4-8 h is required to localize in the targeted area and clear from other background tissues and organs. Organic fluorophores used as contrast agents in those processes are light-sensitive, and prolonged exposure to light can induce photodegradation. Photobleaching is an outcome of the photodegradation process, which may cause unwanted toxicity and harmful effects. Also, it can limit the overall optical applications of the fluorophores during the in vivo process. Photobleaching is typically observed in the long-wavelength cyanine fluorophores because of their long polymethine bridge when they are in solution. Photostability studies were performed on selected fluorophores 18, 19, and 20 (as labeled in Example 4 and FIG. 7D) versus the FDA-approved indocyanine green (ICG) by continuous irradiation with a xenon lamp at 150 W for 2 h. The power density of 150 W is much higher than the required power for fluorescence imaging, considering that the time it takes for a fluorophore to clear from the background and accumulate in sufficient quantities in the targeted tissues is unknown. However, using high-power sources can be justified if the fluorophore remains stable and demonstrates better photostability than ICG under these conditions. This is because a fluorophore, which showed better photostability by enduring a high light source would exhibit less photobleaching if there is a low energy source throughout the process. By considering this, an aliquot of the stock solution (1 mM in DMSO) was diluted in ethanol, and the fluorescence intensity was measured at the excitation wavelength same as their absorption maxima (nm). Photodegradation rates were measured every 20 min intervals for the selected fluorophores based on the % reduced fluorescence intensity starting from 100%. In dark conditions at room temperature, the selected fluorophores showed no photobleaching for over 24 h, while in the presence of light, all of the fluorophores showed photodegradation. Obtained results are presented in FIG. 24. The data were collected in every 20 min intervals under continuous irradiation with a xenon lamp at 150 W for 2 h and presented based on the % reduced fluorescence intensity starting from 100%. All of the tested fluorophores showed better stability than ICG possibly due to their rigid structure with fewer rotatable bonds, which reduces their susceptibility to chemical and photodegradation. In contrast, ICG has a more flexible structure with more rotatable bonds (14 nrotb), making it more prone to degradation over time under exposure to light. Fluorophore 18 degraded I-4%, and fluorophores 20 and 19 degraded 7-12, while ICG degraded 41% at the end of 2 h. The photostability of the fluorophores decreases in the order 18>20>19>ICG, which demonstrates that the presence of cyclohexenyl rings in fluorophores 18, 19, and 20 in the polymethine bridge showed increased photostability.

### Example 9—Biodistribution Study with Exemplified Compounds

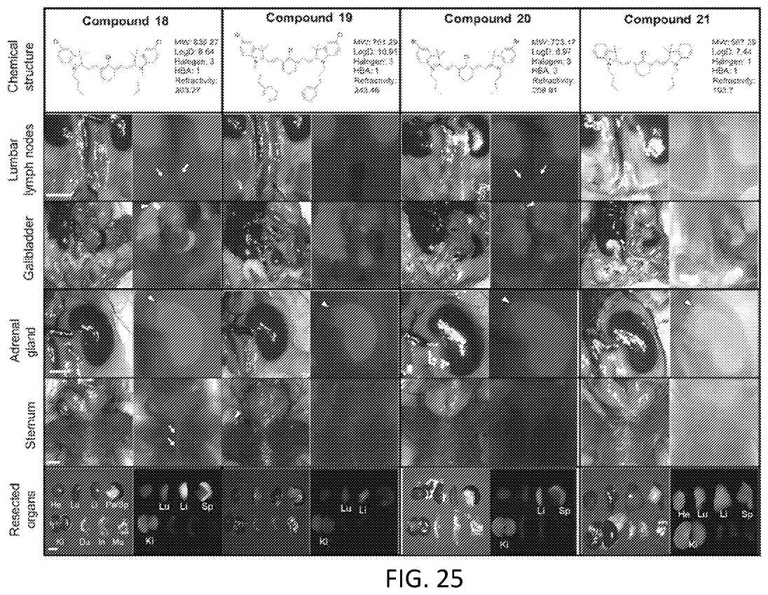

Synthesized fluorophores 18-21 (as labeled in Example 4 and FIG. 7D) were injected into CD-1 male mice to study their biodistribution and tissue/organ-targeting characteristics. 4 h prior to sequential intraoperative imaging, 25 nmol of each fluorophore was injected intravenously. As shown in FIG. 25, heptamethine fluorophores 18 and 20, containing butyl chains on nitrogen atoms of heterocyclic backbone exhibited high signals in the primary and secondary lymphoid tissues including the bone marrow, spleen, lymph nodes, liver, gallbladder, and adrenal glands. The liver and gallbladder signals were consistent with hepatobiliary clearance. Chlorine-substituted fluorophore 18 produced higher signals in the bone marrow, spleen, and lymph nodes compared to bromine-substituted fluorophore 20. However, fluorophore 21, without halogens on the sides, showed high background signals due to the serum protein binding during systemic circulation.

In FIG. 25, abbreviations used are Du, duodenum; He, heart; In, intestine; Ki, kidney; Li, liver; Lu, lung; Mu, muscle; Pa, pancreas; and Sp, spleen. Arrows and arrowheads indicate the targeted organs.

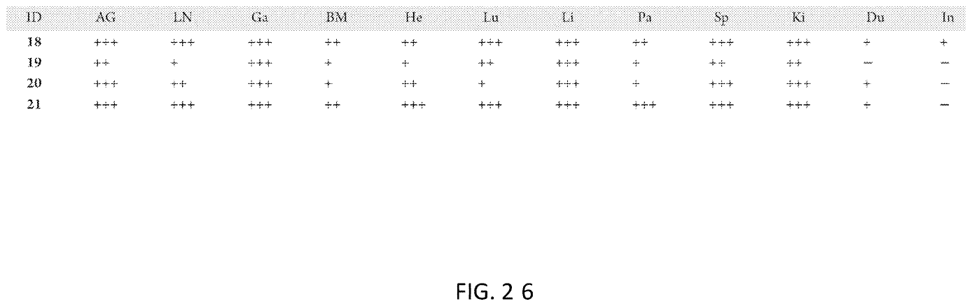

FIG. 26 shows the summary of targeting properties and biodistribution of heptamethine fluorophores. Overall, the meso-chlorinated heptamethine fluorophore 21 showed higher background tissue uptake with circulation in the bloodstream for a longer period of time compared to other substitutions due to the rapid binding to serum proteins.

In FIG. 26, abbreviations used are AG, adrenal gland; BM, bone marrow; Du, duodenum; Ga, gallbladder; He, heart; In, intestine; Ki, kidney; Li, liver; Lu, lung; LN, lymph node; Pa, pancreas; and Sp, spleen. Arrows and arrowheads indicate the targeted organs. The SBR of each organ/tissue relative to the muscle was quantified and labeled as −, 1 to 2; +, 2 to 3; ++, 3 to 5; and +++, >5.

### OTHER EMBODIMENTS

It is to be understood that while the present application has been described in conjunction with the detailed description thereof, the foregoing description is intended to illustrate and not limit the scope of the present application, which is defined by the scope of the appended claims. Other aspects, advantages, and modifications are within the scope of the following claims.

1. A compound of Formula (I): 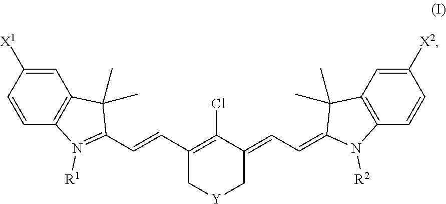
or a pharmaceutically acceptable salt thereof, wherein:
Y is selected from CH₂, O, S, and CHOR₃;
X¹ is selected from H, halo, C₁₋₃ alkyl, C₁₋₃ haloalkyl, OH, C₁₋₃ alkoxy, C₁₋₃ haloalkoxy, NH₂, C₁₋₃ alkylamino, and di(C₁₋₃ alkyl)amino;
X² is selected from H, halo, C₁₋₃ alkyl, C₁₋₃ haloalkyl, OH, C₁₋₃ alkoxy, C₁₋₃ haloalkoxy, NH₂, C₁₋₃ alkylamino, and di(C₁₋₃ alkyl)amino;
R¹ is selected from C₁₋₈ alkyl and C₁₋₈ haloalkyl, wherein said C₁₋₈ alkyl is optionally substituted with OH, C₁₋₃ alkoxy, C(═O)OH, SO₃H, P(═O)(OH)₂, NH₂, C₁₋₃ alkylamino, di(C₁₋₃ alkyl)amino, tri(C₁₋₃ alkyl)amino, C₆₋₁₀ aryl, or 5-10 membered heteroaryl;
R² is selected from C₁₋₈ alkyl and C₁₋₈ haloalkyl, wherein said C₁₋₈ alkyl is optionally substituted with OH, C₁₋₃ alkoxy, C(═O)OH, SO₃H, P(═O)(OH)₂, NH₂, C₁₋₃ alkylamino, di(C₁₋₃ alkyl)amino, tri(C₁₋₃ alkyl)amino, C₆₋₁₀ aryl, or 5-10 membered heteroaryl; and
R³ is selected from C₁₋₈ alkyl and C₂₋₄ alkynyl.

2. The compound of claim 1, wherein Y is O or S.

3. The compound of claim 1, wherein the compound of Formula (I) has formula: 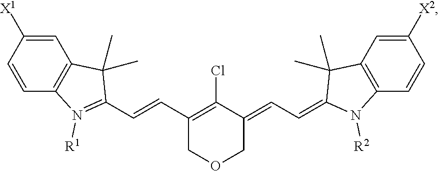
or a pharmaceutically acceptable salt thereof.

4. The compound of claim 1, wherein the compound of Formula (I) has formula: 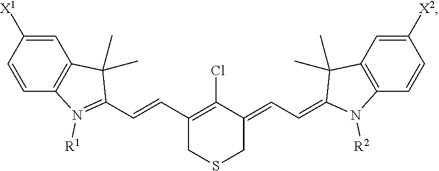
or a pharmaceutically acceptable salt thereof.

5. The compound of claim 1, wherein X¹ is selected from H, halo, OH, methoxy, and NH₂.

6. The compound of claim 5, wherein X¹ is halo.

7. The compound of claim 6, wherein X¹ is Cl, F, or Br.

8. The compound of claim 1, wherein X² is selected from H, halo, OH, methoxy, and NH₂.

9. The compound of claim 8, wherein X² is halo.

10. The compound of claim 9, wherein X² is Cl, F, or Br.

11. The compound claim 1, wherein:
X¹ is selected from halo, C₁₋₃ alkyl, C₁₋₃ haloalkyl, OH, C₁₋₃ alkoxy, C₁₋₃ haloalkoxy, NH₂, C₁₋₃ alkylamino, and di(C₁₋₃ alkyl)amino; and
X² is selected from halo, C₁₋₃ alkyl, C₁₋₃ haloalkyl, OH, C₁₋₃ alkoxy, C₁₋₃ haloalkoxy, NH₂, C₁₋₃ alkylamino, and di(C₁₋₃ alkyl)amino.

12. The compound of claim 1, wherein:
X¹ is halo; and
X² is halo.

13. The compound of claim 12, wherein:
X¹ is Cl, F, or Br; and
X² is Cl, F, or Br.

14. The compound of claim 13, wherein:
X¹ is Cl; and
X² is Cl.

15. The compound of claim 1, wherein the compound of Formula (I) has formula: 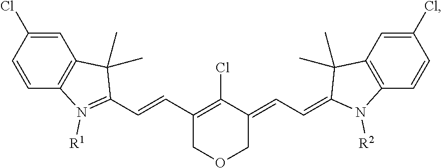
or a pharmaceutically acceptable salt thereof.

16. The compound of claim 1, wherein the compound of Formula (I) has formula: 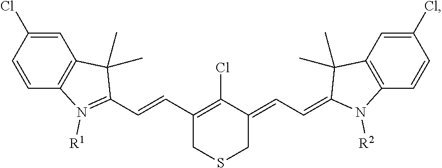
or a pharmaceutically acceptable salt thereof.

17. The compound of claim 1, wherein R¹ is C₁₋₈ alkyl, optionally substituted with OH, C(═O)OH, SO₃H, or C₆₋₁₀ aryl.

18. The compound of claim 17, wherein R¹ is C₁₋₆ alkyl.

19. The compound of claim 1, wherein R² is C₁₋₈ alkyl, optionally substituted with OH, C(═O)OH, SO₃H, or C₆₋₁₀ aryl.

20. The compound of claim 19, wherein R² is C₁₋₆ alkyl.

21. The compound of claim 1, wherein:
R¹ is C₁₋₆ alkyl, optionally substituted with C₆₋₁₀ aryl; and
R² is C₁₋₆ alkyl, optionally substituted with C₆₋₁₀ aryl.

22. The compound of claim 1, wherein the compound of Formula (I) is selected from any one of the following compounds: 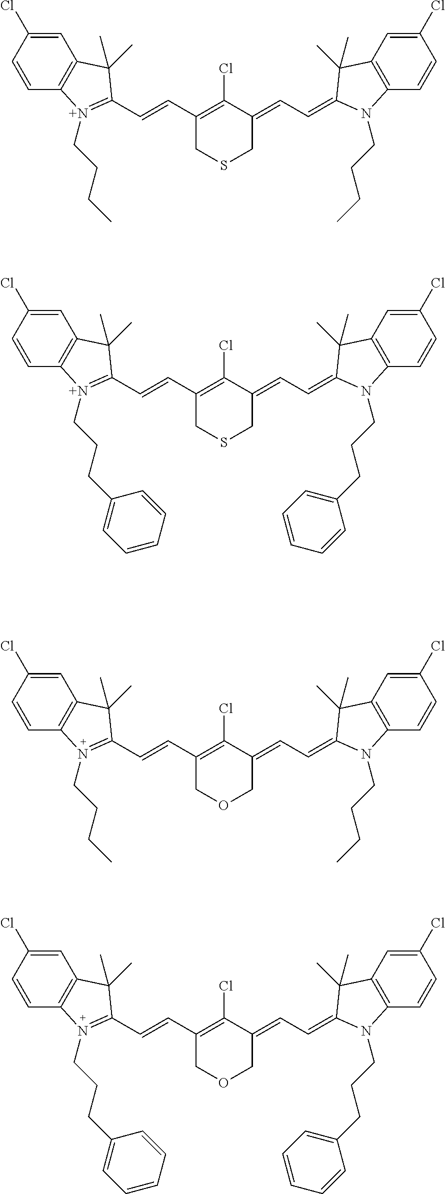 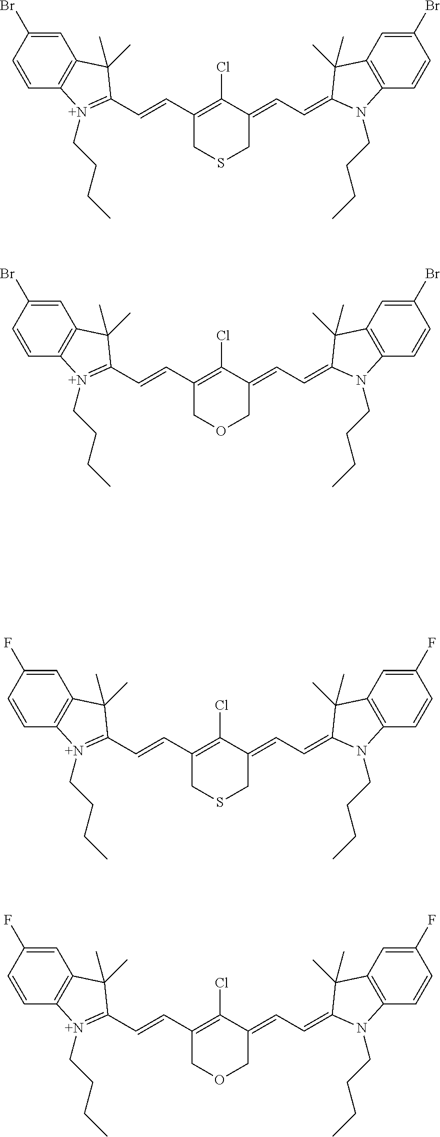 [SMILES: CCCCN1/C(=C/C=C2\CSCC(/C=C/C3=[N+](CCCC)c4ccc(Br)cc4C3(C)C)=C2Cl)C(C)(C)c2cc(Br)ccc21 ; CCCCN1/C(=C/C=C2\COCC(/C=C/C3=[N+](CCCC)c4ccc(Br)cc4C3(C)C)=C2Cl)C(C)(C)c2cc(Br)ccc21 ; CCCCN1/C(=C/C=C2\CSCC(/C=C/C3=[N+](CCCC)c4ccc(F)cc4C3(C)C)=C2Cl)C(C)(C)c2cc(F)ccc21 ; CCCCN1/C(=C/C=C2\COCC(/C=C/C3=[N+](CCCC)c4ccc(F)cc4C3(C)C)=C2Cl)C(C)(C)c2cc(F)ccc21] 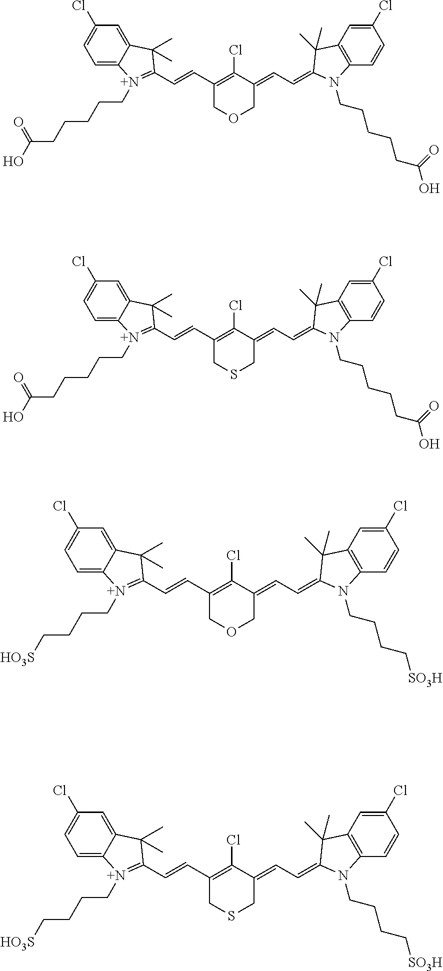 [SMILES: CC1(C)C(/C=C/C2=C(Cl)C(=C/C=C3/N(CCCCCC(=O)O)c4ccc(Cl)cc4C3(C)C)/COC2)=[N+](CCCCCC(=O)O)c2ccc(Cl)cc21 ; CC1(C)C(/C=C/C2=C(Cl)C(=C/C=C3/N(CCCCCC(=O)O)c4ccc(Cl)cc4C3(C)C)/CSC2)=[N+](CCCCCC(=O)O)c2ccc(Cl)cc21 ; CC1(C)C(/C=C/C2=C(Cl)C(=C/C=C3/N(CCCCS(=O)(=O)O)c4ccc(Cl)cc4C3(C)C)/COC2)=[N+](CCCCS(=O)(=O)O)c2ccc(Cl)cc21 ; CC1(C)C(/C=C/C2=C(Cl)C(=C/C=C3/N(CCCCS(=O)(=O)O)c4ccc(Cl)cc4C3(C)C)/CSC2)=[N+](CCCCS(=O)(=O)O)c2ccc(Cl)cc21] 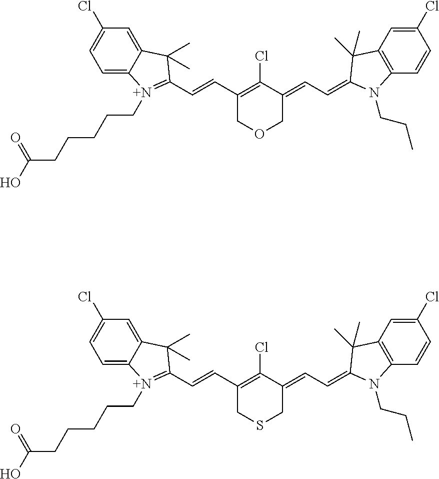 [SMILES: CCCN1/C(=C/C=C2\COCC(/C=C/C3=[N+](CCCCCC(=O)O)c4ccc(Cl)cc4C3(C)C)=C2Cl)C(C)(C)c2cc(Cl)ccc21 ; CCCN1/C(=C/C=C2\CSCC(/C=C/C3=[N+](CCCCCC(=O)O)c4ccc(Cl)cc4C3(C)C)=C2Cl)C(C)(C)c2cc(Cl)ccc21]
or a pharmaceutically acceptable salt thereof.

23. The compound of claim 1, wherein Y is CHOR₃.

24. The compound of claim 23, wherein R³ is propargyl.

25. The compound of claim 23, wherein:
X¹ is H, F, Cl, or Br;
X² is H, F, Cl, or Br;
R¹ is ethyl or butyl; and
R² is ethyl or butyl.

26. The compound of claim 1, wherein the compound of Formula (I) is selected from any one of the following compounds: 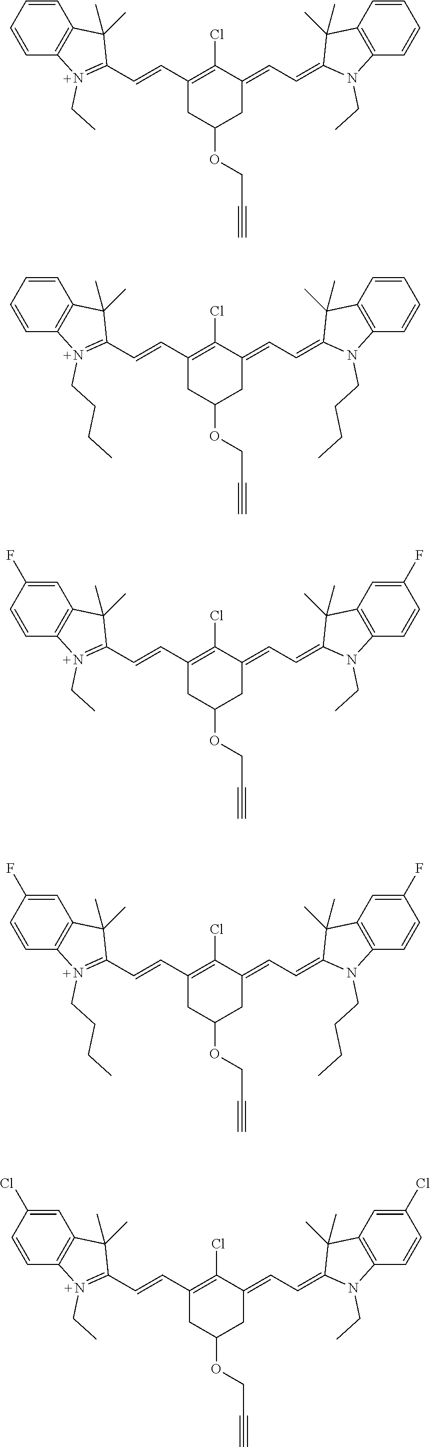 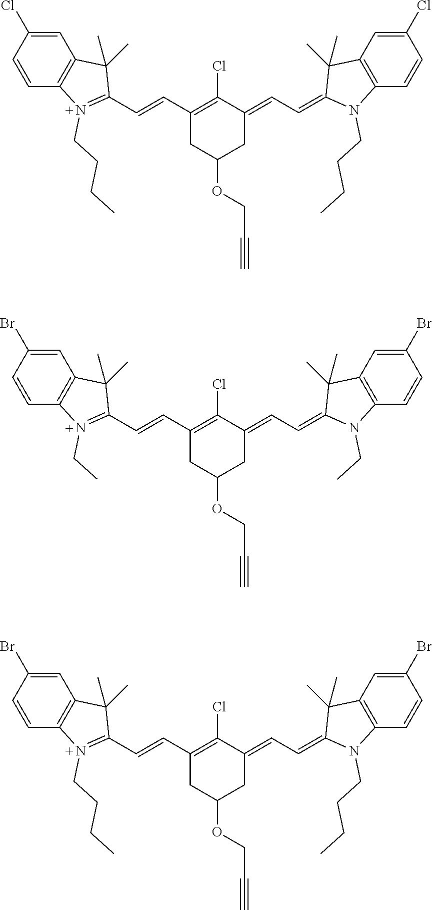
or a pharmaceutically acceptable salt thereof.

27. A pharmaceutical composition comprising a compound of claim 1, or a pharmaceutically acceptable salt thereof, and a pharmaceutically acceptable carrier.

28. A method of imaging a cancerous tumor in a subject, the method comprising:
i) administering to the subject an effective amount of a compound of claim 1, or a pharmaceutically acceptable salt thereof;
ii) waiting a time sufficient to allow the compound to accumulate in the cancerous tumor to be imaged; and
iii) imaging the cancerous tumor with a fluorescence imaging technique.

29. The method of claim 28, wherein the fluorescence imaging technique is NIR-II fluorescence imaging.

30. The method of claim 28, wherein the time sufficient to allow the compound to accumulate in the cancerous tumor is from about 24 hours to about 168 hours.

31. A method of treating cancer, the method comprising:
i) imaging a cancerous tumor in a subject according to the method of claim 28; and
ii) surgically removing the cancerous tumor from the subject.
